(19) United States
(12) Patent Application Publication
(10) Pub. No.: US 2017/0268066 A1
(43) Pub. Date: Sep. 21, 2017

(54) CANCER BIOMARKERS

(71) Applicant: Chalmers Ventures AB, Gothenburg (SE)

(72) Inventors: Francesco GATTO, San Diego, CA (US); Jens NIELSEN, Gothenburg (SE); Almut SCHULZE, Wurzburg (DE)

(73) Assignee: Chalmers Ventures AB, Gothenburg (SE)

(21) Appl. No.: 15/458,125

(22) Filed: Mar. 14, 2017

Related U.S. Application Data
(60) Provisional application No. 62/308,635, filed on Mar. 15, 2016.

(51) Int. Cl. C12Q 1/68; G01N 33/574

(52) U.S. Cl. CPC C12Q 1/6886; G01N 33/574; C12Q 2600/158; C12Q 2600/156; C12Q 2600/118

(57) ABSTRACT

The present invention relates to a method of screening for cancer in a subject, said method comprising determining the level in a sample of an expression product of one or more genes of a certain metabolic network of reactions and/or determining the level in a sample of a metabolite related to an expression product of one or more of said genes, wherein said sample has been obtained from said subject. Methods of treating cancer and kits are also provided.

The present invention relates generally to biomarkers for cancer and to methods of screening for cancer. Such methods involve determining the level of certain biomarkers which are indicative of cancer in a subject.

Sequencing of an increasing number of cancer genomes has revealed the extent of genomic heterogeneity of the disease, which stems from a complex interplay of mutations and the natural selection of clones (Yates, L. R., and Campbell, P. J. (2012) Nature reviews. Genetics 13, 795-806). The complexity of the cancer genome is a daunting challenge for the rational treatment of the disease. While progress has been made in the attempt to tailor treatments to the defined molecular features of individual tumors, the need for ever more precise patient stratification provides a rational limit for these strategies (Chin, L., Andersen, J. N., and Futreal, P. A. (2011) Nature medicine 17, 297-303). Moreover, the concept of convergent evolution in cancer could explain the acquisition of the cancer phenotype through multiple routes (Gerlinger, M., et al. (2014) Annu Rev Genet 48, 215-236; Hanahan, D., and Weinberg, R. A. (2011) Cell 144, 646-674; Weinberg, R. A. (2014) Cell 157, 267-271).

Mutations are central in the evolution of most cancers and, once acquired, they are liabilities that cancers carry throughout their progression. In addition to direct effects on cellular signaling networks and the reprogramming of gene expression, cancer mutations also initiate a process of natural selection, which results in the emergence of cell lineages exhibiting the transformed characteristic of cancer (Vogelstein, B., et al. (2013). Science 339, 1546-1558). It is conceivable to factorize the expression level of each gene as the contribution of different tumor features, and extract the contribution due to occurrence of a cancer mutation. In turn, common transcriptional changes attributable to different mutations, i.e. convergence towards a common set of deregulated genes, should correspond to the deregulation of biological processes crucial for cancer evolution. These key processes are then selected for via mutagenesis and natural selection, and define the phenotype of cancer.

The present inventors have found that mutations in certain genes that are commonly mutated in cancer are associated with substantial changes in gene expression which primarily converge on a metabolic network of reactions, referred to herein as the AraX network (or AraX pathway), that involve the glutathione- and oxygen-mediated metabolism of arachidonic acid and xenobiotics. The AraX network comprises 84 genes (referred to herein as ‘AraX genes’). Screening for the deregulation of the AraX network, for example for an alteration in the level of one or more the expression products (and/or related metabolites) of this metabolic network of reactions thus represents an advantageous method of screening for cancer in a subject.

Thus, in a first aspect the present invention provides a method of screening for cancer in a subject, said method comprising determining the level in a sample of an expression product of one or more genes selected from the group consisting of:

ADH1C, FAAH2, MBOAT2, PLA2G2A, PLA2G4A, PLA2G4E, PLA2G10, ELOVL2, CYP2S1, CYP4F11, AKR1C3, CBR1, GSTM2, GSTM3, HPGDS, HPGD, PTGS1, PTGES, ALOX15, CYP4F3, GGT6, PTGR1, GCLC, GCLM, GPX2, GPX3, GSR, OPLAH, CYP2W1, CYP4B1, CYP4X1, CYP24A1, CYP27A1, CYP27B1, CYP39A1, HGD, MOXD1, CDO1, CP, CYP3A5, ADH6, ADH7, ADHFE1, FMO3, FMO4, FMO5, AKR1B15, AKR1B10, AKR1C1, AKR1C2, NQO1, NQO2, CBR3, ALDH3A1, ALDH3A2, ALDH3B1, AOC1, MAOB, CES1, EPHX1, GSTA2, GSTM1, GSTM4, MGST1, UGT1A1, UGT1A6, SULT1A1, SULT1A2, SULT1A4, ACSL5, SLCO1B3, SLCO2A1, ABCC1, ABCC2, ABCC3, ALOX5, CYP2E1, LTC4S, PLA2G6, PLA2G12A, PTGS2, GSTO1, GSTO2 and FMO1

and/or

determining the level in a sample of a metabolite related to an expression product of one or more of said genes;
wherein said sample has been obtained from said subject; and

wherein an altered level in said sample of the expression product of one or more of said genes and/or of a metabolite related to an expression product of one or more of said genes in comparison to a control level is indicative of cancer in said subject.

In another aspect, the present invention provides a method of screening for cancer in a subject, said method comprising

determining the level in a sample of an expression product of one or more genes selected from the group consisting of:

ADH1C, FAAH2, MBOAT2, PLA2G2A, PLA2G4A, PLA2G4E, PLA2G10, ELOVL2, CYP2S1, CYP4F11, AKR1C3, CBR1, GSTM2, GSTM3, HPGDS, HPGD, PTGS1, PTGES, ALOX15, CYP4F3, GGT6, PTGR1, GCLC, GCLM, GPX2, GPX3, GSR, OPLAH, CYP2W1, CYP4B1, CYP4X1, CYP24A1, CYP27A1, CYP27B1, CYP39A1, HGD, MOXD1, CDO1, CP, CYP3A5, ADH6, ADH7, ADHFE1, FMO3, FMO4, FMO5, AKR1B15, AKR1B10, AKR1C1, AKR1C2, NQO1, NQO2, CBR3, ALDH3A1, ALDH3A2, ALDH3B1, AOC1, MAOB, CES1, EPHX1, GSTA2, GSTM1, GSTM4, MGST1, UGT1A1, UGT1A6, SULT1A1, SULT1A2, SULT1A4, ACSL5, SLCO1B3, SLCO2A1, ABCC1, ABCC2, ABCC3, ALOX5, CYP2E1, LTC4S, PLA2G6, PLA2G12A, PTGS2, GSTO1, GSTO2 and FMO1

and/or

determining the level in a sample of a metabolite related to an expression product of one or more of said genes;

wherein said sample has been obtained from said subject.

In preferred embodiments, the level in a sample of an expression product of one or more AraX genes is determined.

In some embodiments, the level in a sample of a metabolite related to an expression product of one or more AraX genes is determined.

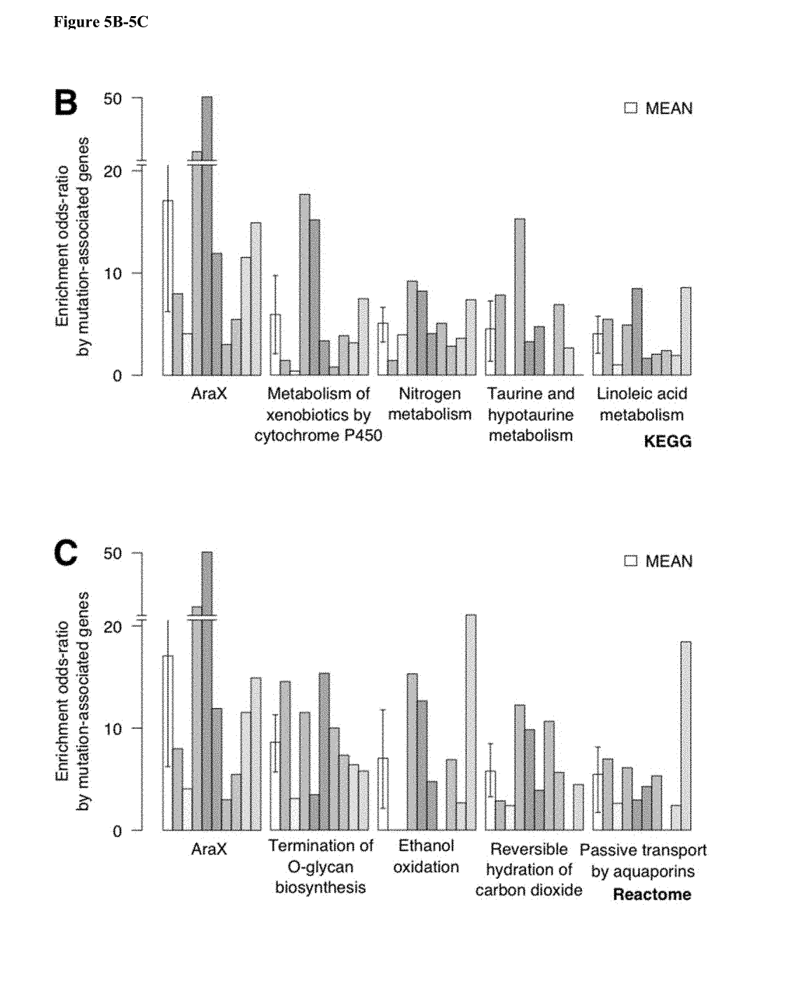

In one embodiment, the method comprises determining the level in a sample of an expression product of one or more genes involved in the metabolism of arachidonic acid (genes of the arachidonic acid metabolic pathway), or of a metabolite related to an expression product of one or more of said genes. Such genes are described in the Example and the Figures. FIG. 5 depicts the arachidonic acid metabolic pathway.

Thus, in one embodiment, the method comprises determining the level in a sample of an expression product of one or more genes selected from the group consisting of FAAH2, PLA2G2A, PLA2G4A, PLA2G4E, PLA2G6, PLA2G10, PLA2G12A, MBOAT2, ELOVL2, CYP2E1, CYP2S1, CYP4F11, ALOX5, ALOX15, PTGS1, PTGS2, PTGR1, CYP4F3, LTC4S, GGT6, AKR1C3, HPGDS, PTGES, GSTM2, GSTM3, CBR1 and HPGD, or of a metabolite related to an expression product of one or more of said genes.

In one embodiment, the method comprises determining the level in a sample of an expression product of one or more genes selected from the group consisting of CYP2S1 and CYP4X1, or of a metabolite related to an expression product of one or more of said genes.

In one embodiment, the method comprises determining the level in a sample of an expression product of one or more genes selected from the group consisting of PTGS1, PTGES, GSTM2, GSTM3, CBR1, HPGDS, AKR1C3 and HPDG, or of a metabolite related to an expression product of one or more of said genes. These genes are components of the arachidonic acid metabolic pathway and are each involved in the metabolism of prostaglandin H₂.

In one embodiment, the method comprises determining the level in a sample of an expression product of one or more genes selected from the group consisting of CYP4F3, PTGR1 and GGT6, or of a metabolite related to an expression product of one or more of said genes. These genes are components of the arachidonic acid metabolic pathway and are each involved in the metabolism of leukotriene B₄ and C₄.

In one embodiment, the method comprises determining the level in a sample of an expression product of one or more genes selected from the group consisting of PLA2G2A, PLA2G4A, PLA2G4E and PLA2G10, or of a metabolite related to an expression product of one or more of said genes. These genes are components of the arachidonic acid metabolic pathway and each belong to the class of phospholipases A₂.

In one embodiment, the method comprises determining the level in a sample of an expression product of one or more genes involved in the metabolism of xenobiotics (genes of the xenobiotic metabolic pathway), or of a metabolite related to an expression product of one or more of said genes. Such genes are described in the Example and the Figures. FIG. 5 depicts the xenobiotic metabolic pathway.

Thus, in one embodiment, the method comprises determining the level in a sample of an expression product of one or more genes selected from the group consisting of CYP3A5, ALDH3A1, ALDH3A2, ALDH3B1, NQO1, NQO2, AOC1, MAOB, CBR3, CES1, EPHX1, ADH1C, ADH6, ADH7, ADHFE1, AKR1B15, AKR1B10, AKR1C1, AKR1C2, FMO1, FMO3, FMO4, FMO5, GSTA2, GSTM1, GSTM4, GSTO1, GSTO2, MGST1, ACSL5, UGT1A1, UGT1A6, SULT1A1, SULT1A2 and SULT1A4, or of a metabolite related to an expression product of one or more of said genes.

In one embodiment, the method comprises determining the level in a sample of an expression product of one or more genes selected from the group consisting of CYP3A5, ADH1C, ADH6, ADH7, ADHFE1, FMO3, FMO4, FMO5, AKR1B10, AKR1B15, AKR1C1, AKR1C2, NQO1, NQO2, CBR3, ALDH3A1, ALDH3A2, ALDH3B1, AOC1, MAOB, CES1 and EPHX1, or of a metabolite related to an expression product of one or more of said genes. These genes are components of the xenobiotic metabolic pathway and are each implicated in phase I of xenobiotic metabolism (also called functionalization).

In one embodiment, the method comprises determining the level in a sample of an expression product of one or more genes selected from the group consisting of CYP3A5, ADH1C, ADH6, ADH7, ADHFE1, FMO3, FMO4, FMO5, AKR1B10, AKR1B15, AKR1C1, AKR1C2, NQO1, NQO2, CBR3, ALDH3A1, ALDH3A2, ALDH3B1, AOC1 and MAOB, or of a metabolite related to an expression product of one or more of said genes. These genes are components of the xenobiotic metabolic pathway and are oxidoreductases.

In one embodiment, the method comprises determining the level in a sample of an expression product of one or more genes selected from the group consisting of ADH1C, ADH6, ADH7 and ADHFE1, or of a metabolite related to an expression product of one or more of said genes. These genes are components of the xenobiotic metabolic pathway and are alcohol dehydrogenases.

In one embodiment, the method comprises determining the level in a sample of an expression product of one or more genes selected from the group consisting of FMO3, FMO4 and FMO5, or of a metabolite related to an expression product of one or more of said genes. These genes are components of the xenobiotic metabolic pathway and are flavin-containing monooxygenases.

In one embodiment, the method comprises determining the level in a sample of an expression product of one or more genes selected from the group consisting of AKR1B10, AKR1B15, AKR1C1 and AKR1C2, or of a metabolite related to an expression product of one or more of said genes. These genes are components of the xenobiotic metabolic pathway and are aldo-keto reductases.

In one embodiment, the method comprises determining the level in a sample of an expression product of one or more genes selected from the group consisting of NQO1 and NQO2, or of a metabolite related to an expression product of one or more of said genes. These genes are components of the xenobiotic metabolic pathway and are quinone reductases.

In one embodiment, the method comprises determining the level in a sample of an expression product of one or more genes selected from the group consisting of ALDH3A1, ALDH3A2 and ALDH3B1, or of a metabolite related to an expression product of one or more of said genes. These genes are components of the xenobiotic metabolic pathway and are aldehyde dehydrogenases.

In one embodiment, the method comprises determining the level in a sample of an expression product of one or more genes selected from the group consisting of AOC1 and MAOB, or of a metabolite related to an expression product of one or more of said genes. These genes are components of the xenobiotic metabolic pathway and are amine oxidases.

In one embodiment, the method comprises determining the level in a sample of an expression product of one or more genes selected from the group consisting of CES1 and EPHX1, or of a metabolite related to an expression product of one or more of said genes. These genes are components of the xenobiotic metabolic pathway and are hydrolases.

In one embodiment, the method comprises determining the level in a sample of an expression product of one or more genes selected from the group consisting of UGT1A1, UGT1A6, GSTA2, GSTM1, GSTM4, MGST1, SULT1A1, SULT1A2, SULT1A4 and ACSL5, or of a metabolite related to an expression product of one or more of said genes. These genes are components of the xenobiotic metabolic pathway and are each implicated in phase II of xenobiotic metabolism (also called conjugation).

In one embodiment, the method comprises determining the level in a sample of an expression product of one or more genes selected from the group consisting of UGT1A1 and UGT1A6, or of a metabolite related to an expression product of one or more of said genes. These genes are components of the xenobiotic metabolic pathway and are UDPGA transferases that carry out glucuronidation reactions on xenobiotics.

In one embodiment, the method comprises determining the level in a sample of an expression product of one or more genes selected from the group consisting of GSTA2, GSTM1, GSTM4 and MGST1, or of a metabolite related to an expression product of one or more of said genes. These genes are components of the xenobiotic metabolic pathway and catalyse the conjugation of glutathione.

In one embodiment, the method comprises determining the level in a sample of an expression product of one or more genes selected from the group consisting of SULT1A1, SULT1A2 and SULT1A4, or of a metabolite related to an expression product of one or more of said genes. These genes are components of the xenobiotic metabolic pathway and are sulfotransferases and are responsible for sulfonation reactions on xenobiotics using PAPS as cofactor.

In one embodiment, the method comprises determining the level in a sample of an expression product of one or more genes selected from the group consisting of AKR1C3, CBR1, GSTM2, GSTM3, CYP2E1, CYP2S1 and CYP4F11 or of a metabolite related to an expression product of one or more of said genes. In addition to being components of the arachidonic acid metabolic pathway, these genes are also reported to have activity in the detoxification of electrophilic xenobiotics, e.g. by oxidising xenobiotics.

In one embodiment, the method comprises determining the level in a sample of an expression product of one or more genes that encode transporters for arachidonic acid-derived products and solubilized xenobiotics, or of a metabolite related to an expression product of one or more of said genes. Such genes are described in the Example and the Figures. FIG. 5 depicts these transporters.

Thus, in one embodiment, the method comprises determining the level in a sample of an expression product of one or more genes selected from the group consisting of SLCO2A1, SLCO1B3, ABCC1, ABCC2 and ABCC3, or of a metabolite related to an expression product of one or more of said genes.

In one embodiment, the method comprises determining the level in a sample of an expression product of one or more genes selected from the group consisting of SLCO2A1 and SLCO1B3, or of a metabolite related to an expression product of one or more of said genes. These genes are organic anion transporters that show affinity for prostaglandin D₂ and leukotriene C₄, respectively.

In one embodiment, the method comprises determining the level in a sample of an expression product of one or more genes selected from the group consisting of ABCC1, ABCC2 and ABCC3, or of a metabolite related to an expression product of one or more of said genes. These genes are ABC transporters able to move a variety of xenobiotics and other substrates include prostaglandin A₁, A₂, D₂, E₂, 15d J₂ and leukotriene C₄.

In one embodiment, the method comprises determining the level in a sample of an expression product of one or more genes involved in oxygen- or glutathione consuming reactions, or of a metabolite related to an expression product of one or more of said genes. Such genes are described in the Example and the Figures. FIG. 5 depicts the xenobiotic metabolic pathway.

Thus, in one embodiment, the method comprises determining the level in a sample of an expression product of one or more genes selected from the group consisting of GCLC, GCLM, GPX2, GPX3, GSR, OPLAH, CYP2W1, CYP4B1, CYP4X1, CYP24A1, CYP27A1, CYP27B1, CYP39A1, HGD, MOXD1, CDO1 and CP, or of a metabolite related to an expression product of one or more of said genes.

In one embodiment, the method comprises determining the level in a sample of an expression product of one or more genes selected from the group consisting of GPX2 and GPX3, or of a metabolite related to an expression product of one or more of said genes. These genes are involved in glutathione metabolism.

In one embodiment, the method comprises determining the level in a sample of an expression product of one or more genes selected from the group consisting of GCLC, GCLM, GSR and OPLAH, or of a metabolite related to an expression product of one or more of said genes. These genes are involved in glutathione biosynthesis.

In one embodiment, the method comprises determining the level in a sample of an expression product of one or more genes selected from the group consisting of CYP2W1, CYP4B1, CYP4X1, CYP24A1, CYP27A1, CYP27B1 and CYP39A1, or of a metabolite related to an expression product of one or more of said genes. These genes are members of the cytochrome P450 family.

In one embodiment, the method comprises determining the level in a sample of an expression product of one or more genes selected from the group consisting of ALOX5, LTC4S CYP2E1, PLA2G6, PLA2G12A, PTGS2, PTGS1, FMO1 and GSTO1 and GSTO2, or of a metabolite related to an expression product of one or more of said genes.

In one embodiment, the method comprises determining the level in a sample of an expression product of one or more genes selected from the group consisting of HGD, ADH7 and ALDH3A1, or of a metabolite related to an expression product of one or more of said genes.

In one embodiment, the method comprises determining the level in a sample of an expression product of one or more genes selected from the group consisting of HGD and ADH7, or of a metabolite related to an expression product of one or more of said genes.

In one embodiment, the method comprises determining the level in a sample of an expression product of one or more genes selected from the group consisting of ADH7, GSTM3, ABCC2, PTGR1 and CBR3.

In one embodiment, the method comprises determining the level in a sample of an expression product of one or more genes involved in protein glycosylation, or of a metabolite related to an expression product of one or more of said genes

In a preferred embodiment, the method comprises determining the level in a sample of an expression product of ADH1C or of a metabolite related to ADH1C.

In a preferred embodiment, the method comprises determining the level in a sample of an expression product of GPX3 or of a metabolite related to GPX3.

In a preferred embodiment, the method comprises determining the level in a sample of an expression product of CDO1 or of a metabolite related to CDO1.

In one embodiment, the method comprises determining the level in a sample of an expression product of HGD, or of a metabolite related to HGD.

In some embodiments of the present invention the level of an expression product of one or more of the following genes is not determined: PLA2G2A, PLA2G4A, PTGS2, GSR, NQO2.

In some embodiments of the present invention the level of an expression product of ADH7 is not determined.

As discussed herein, methods of the present invention may comprise determining or measuring the level of an expression product of one or more specific genes (or groups of genes) “selected from the group consisting of” certain specific genes (or groups of genes) set forth herein. For the avoidance of doubt, in some embodiments in which one or more of the specific genes (or groups of genes) discussed herein is measured or determined, one or more other (or distinct) genes and/or one or more other biomarkers may additionally be measured or determined. Thus, “selected from the group consisting of” may be an “open” term. In some embodiments, only one or more of the specific genes (or groups of genes) discussed herein is measured or determined (e.g. other genes or other biomarkers are not measured or determined).

An altered level of one or more of the expression products or metabolites as described herein includes any measurable alteration or change in level of the expression product or metabolite in question when the expression product or metabolite in question is compared with a control level. An altered level includes an increased or decreased level. Preferably, the level is significantly altered, compared to the level found in an appropriate control sample or subject. More preferably, the significantly altered levels are statistically significant, preferably with a probability value of <0.05. Exemplary altered levels are discussed elsewhere herein in relation to “increased” and “decreased” levels.

In methods of the present invention, it is not necessary that the level of each one of the expression products (or metabolites) whose level is determined is altered in comparison to a control level in order for there to be an indication of cancer in the subject. Put another way, a sample in which the level of one or more expression products (or metabolites) is unaltered (or not significantly altered) in comparison with a control level may still be a “cancer” sample (e.g. if the level of one or more of the other AraX genes or related metabolites is altered in comparison to a control level). In some embodiments, an alteration in the level of an expression product (or metabolite) of one or more of the AraX genes is dependent on the presence of a mutation in one or more genes selected from the group consisting of CTNNB1, IDH1, KEAP1, NFE2L2, NSD1, PTEN, RB1, STK11 and TP53. These nine genes are commonly mutated in cancer. By way of example, the expression level of the AraX gene FAAH2 may not be altered in a sample which has a mutation in CTNNB1, but is altered in a sample which has a mutation in STK11 (see Table B in the Example section).

In some embodiments, the sample comprises a mutated version of one or more genes selected from the group consisting of CTNNB1, IDH1, KEAP1, NFE2L2, NSD1, PTEN, RB1, STK11 and TP53.

Thus, in some embodiments the method of the present invention involves determining the presence or absence in the sample of a mutation (e.g. an insertion, deletion or amino acid substitution) in one or more genes selected from the group consisting of CTNNB1, IDH1, KEAP1, NFE2L2, NSD1, PTEN, RB1, STK11 and TP53. Methods for assessing whether or not a mutation is present are known in the art (e.g. by sequencing the relevant expression product and comparing the sequence to the wildtype sequence, or by detecting the presence or absence of a certain sequence motif(s) that is characteristic of a mutated form). In other embodiments, the presence or absence of a mutation (e.g. an insertion, deletion or amino acid substitution) in one or more genes selected from the group consisting of CTNNB1, IDH1, KEAP1, NFE2L2, NSD1, PTEN, RB1, STK11 and TP53 in a subject (e.g. in a biopsy (e.g. tumour sample) from a subject) may be already known prior to performing the method of the present invention. In some embodiments, if a mutation (mutated gene) is present, alteration in the level of an expression product (or metabolite) whose expression is associated with that mutation (mutated gene) is indicative of cancer.

In some embodiments, if a mutation in the CTNNB1 gene is present, an alteration in the level of an expression product of one or more genes selected from the group consisting of PLA2G4A, HPGD, MOXD1, CYP3A5, MAOB, ACSL5, GSTO2, PLA2G2A, PTGS1, GGT6, GPX3, OPLAH, HGD, CP, AKR1B15, AOC1 and FMO1 or of a metabolite related to one or more of said genes is indicative of cancer in said subject.

In some embodiments, if a mutation in the CTNNB1 gene is present, an increase in the level of an expression product of one or more genes selected from the group consisting of PLA2G4A, HPGD, MOXD1, CYP3A5, MAOB, ACSL5 and GSTO2 or of a metabolite related to one or more of said genes is indicative of cancer in said subject.

In some embodiments, if a mutation in the CTNNB1 gene is present, a decrease in the level of an expression product of one or more genes selected from the group consisting of PLA2G2A, PTGS1, GGT6, GPX3, OPLAH, HGD, CP, AKR1B15, AOC1 and FMO1 or of a metabolite related to one or more of said genes is indicative of cancer in said subject.

In some embodiments, if a mutation in the IDH1 gene is present, an alteration in the level of an expression product of one or more genes selected from the group consisting of MBOAT2, FMO3, CYP2E1, GSTO2, ELOVL2, CBR1, CYP27A1, HGD, MOXD1, ALDH3B1, MAOB, ABCC3, ALOX5, LTC4S and FMO1 or of a metabolite related to one or more of said genes is indicative of cancer in said subject.

In some embodiments, if a mutation in the IDH1 gene is present, an increase in the level of an expression product of one or more genes selected from the group consisting of MBOAT2, FMO3, CYP2E1 and GSTO2 or of a metabolite related to one or more of said genes is indicative of cancer in said subject.

In some embodiments, if a mutation in the IDH1 gene is present, a decrease in the level of an expression product of one or more genes selected from the group consisting of ELOVL2, CBR1, CYP27A1, HGD, MOXD1, ALDH3B1, MAOB, ABCC3, ALOX5, LTC4S and FMO1 or of a metabolite related to one or more of said genes is indicative of cancer in said subject.

In some embodiments, if a mutation in the KEAP1 gene is present, an alteration in the level of an expression product of one or more genes selected from the group consisting of CYP4F11, AKR1C3, CBR1, GSTM3, CYP4F3, PTGR1, GCLC, GCLM, GPX2, GSR, CYP24A1, HGD, ADH7, AKR1B15, AKR1B10, AKR1C1, AKR1C2, NQO1, CBR3, ALDH3A1, CES1, EPHX1, UGT1A1, UGT1A6, ABCC1, ABCC2, ABCC3, GSTO1, HPGD, GGT6, CYP4X1, PLA2G6 and FMO1 or of a metabolite related to one or more of said genes is indicative of cancer in said subject.

In some embodiments, if a mutation in the KEAP1 gene is present, an increase in the level of an expression product of one or more genes selected from the group consisting of CYP4F11, AKR1C3, CBR1, GSTM3, CYP4F3, PTGR1, GCLC, GCLM, GPX2, GSR, CYP24A1, HGD, ADH7, AKR1B15, AKR1B10, AKR1C1, AKR1C2, NQO1, CBR3, ALDH3A1, CES1, EPHX1, UGT1A1, UGT1A6, ABCC1, ABCC2, ABCC3 and GSTO1 or of a metabolite related to one or more of said genes is indicative of cancer in said subject.

In some embodiments, if a mutation in the KEAP1 gene is present, a decrease in the level of an expression product of one or more genes selected from the group consisting of HPGD, GGT6, CYP4X1, PLA2G6 and FMO1 or of a metabolite related to one or more of said genes is indicative of cancer in said subject.

In some embodiments, if a mutation in the NFE2L2 gene is present, an alteration in the level of an expression product of one or more genes selected from the group consisting of PLA2G10, CYP4F11, AKR1C3, CBR1, GSTM2, GSTM3, HPGDS, CYP4F3, PTGR1, GCLC, GCLM, GPX2, GSR, CYP39A1, HGD, ADH1C, ADH7, AKR1B15, AKR1B10, AKR1C1, AKR1C2, NQO1, CBR3, ALDH3A1, ALDH3A2, CES1, EPHX1, GSTA2, GSTM1, GSTM4, MGST1, UGT1A1, UGT1A6, SULT1A1, SULT1A2, SLCO1B3, ABCC1, ABCC2, ABCC3, FMO1, PLA2G4E, CYP2W1, CYP24A1, CYP27B1, CDO1, CYP3A5, SLCO2A1 and PTGS2 or of a metabolite related to one or more of said genes is indicative of cancer in said subject.

In some embodiments, if a mutation in the NFE2L2 gene is present, an increase in the level of an expression product of one or more genes selected from the group consisting of PLA2G10, CYP4F11, AKR1C3, CBR1, GSTM2, GSTM3, HPGDS, CYP4F3, PTGR1, GCLC, GCLM, GPX2, GSR, CYP39A1, HGD, ADH1C, ADH7, AKR1B15, AKR1B10, AKR1C1, AKR1C2, NQO1, CBR3, ALDH3A1, ALDH3A2, CES1, EPHX1, GSTA2, GSTM1, GSTM4, MGST1, UGT1A1, UGT1A6, SULT1A1, SULT1A2, SLCO1B3, ABCC1, ABCC2, ABCC3 and FMO1 or of a metabolite related to one or more of said genes is indicative of cancer in said subject.

In some embodiments, if a mutation in the NFE2L2 gene is present, a decrease in the level of an expression product of one or more genes selected from the group consisting of PLA2G4E, CYP2W1, CYP24A1, CYP27B1, CDO1, CYP3A5, SLCO2A1 and PTGS2 or of a metabolite related to one or more of said genes is indicative of cancer in said subject.

In some embodiments, if a mutation in the NSD1 gene is present, an alteration in the level of an expression product of one or more genes selected from the group consisting of CYP2W1, MOXD1, MBOAT2, CYP4F11, AKR1C3, GSTM3, CYP4F3, CYP39A1, CDO1, ADH7, ADHFE1, FMO4, AKR1B10, AKR1C1, AOC1 and CES1 or of a metabolite related to one or more of said genes is indicative of cancer in said subject.

In some embodiments, if a mutation in the NSD1 gene is present, an increase in the level of an expression product of one or more genes selected from the group consisting of CYP2W1 and MOXD1 or of a metabolite related to one or more of said genes is indicative of cancer in said subject.

In some embodiments, if a mutation in the NSD1 gene is present, a decrease in the level of an expression product of one or more genes selected from the group consisting of MBOAT2, CYP4F11, AKR1C3, GSTM3, CYP4F3, CYP39A1, CDO1, ADH7, ADHFE1, FMO4, AKR1B10, AKR1C1, AOC1 and CES1 or of a metabolite related to one or more of said genes is indicative of cancer in said subject.

In some embodiments, if a mutation in the PTEN gene is present, an alteration in the level of an expression product of one or more genes selected from the group consisting of ALOX15, HGD, NQO1, PTGS2, ABCC2 and CYP2E1 or of a metabolite related to one or more of said genes is indicative of cancer in said subject.

In some embodiments, if a mutation in the PTEN gene is present, an increase in the level of an expression product of one or more genes selected from the group consisting of ALOX15, HGD, NQO1 and PTGS2 or of a metabolite related to one or more of said genes is indicative of cancer in said subject.

In some embodiments, if a mutation in the PTEN gene is present, a decrease in the level of an expression product of one or more genes selected from the group consisting of ABCC2 and CYP2E1 or of a metabolite related to one or more of said genes is indicative of cancer in said subject.

In some embodiments, if a mutation in the RB1 gene is present, an alteration in the level of an expression product of one or more genes selected from the group consisting of ADH7, PLA2G10, GPX3, AKR1C1, AKR1C2, ACSL5, CYP2E1, LTC4S and PLA2G6 or of a metabolite related to one or more of said genes is indicative of cancer in said subject.

In some embodiments, if a mutation in the RB1 gene is present, an increase in the level of an expression product of ADH7 or of a metabolite related to ADH7 is indicative of cancer in said subject.

In some embodiments, if a mutation in the RB1 gene is present, a decrease in the level of an expression product of one or more genes selected from the group consisting of PLA2G10, GPX3, AKR1C1, AKR1C2, ACSL5, CYP2E1, LTC4S and PLA2G6 or of a metabolite related to one or more of said genes is indicative of cancer in said subject.

In some embodiments, if a mutation in the STK11 gene is present, an alteration in the level of an expression product of one or more genes selected from the group consisting of FAAH2, PLA2G4A, PLA2G10, AKR1C3, CBR1, PTGES, GPX3, CYP24A1, HGD, CP, AKR1C1, AKR1C2, NQO1, NQO2, ALDH3B1, AOC1, SULT1A2, SULT1A4, SLCO1B3, ABCC2, PLA2G12A, GSTO2, MBOAT2, CYP2S1, GSTM3 and ADH7 or of a metabolite related to one or more of said genes is indicative of cancer in said subject.

In some embodiments, if a mutation in the STK11 gene is present, an increase in the level of an expression product of one or more genes selected from the group consisting of FAAH2, PLA2G4A, PLA2G10, AKR1C3, CBR1, PTGES, GPX3, CYP24A1, HGD, CP, AKR1C1, AKR1C2, NQO1, NQO2, ALDH3B1, A0C1, SULT1A2, SULT1A4, SLCO1B3, ABCC2, PLA2G12A and GSTO2 or of a metabolite related to one or more of said genes is indicative of cancer in said subject.

In some embodiments, if a mutation in the STK11 gene is present, a decrease in the level of an expression product of one or more genes selected from the group consisting of MBOAT2, CYP2S1, GSTM3 and ADH7 or of a metabolite related to one or more of said genes is indicative of cancer in said subject.

In some embodiments, if a mutation in the TP53 gene is present, an alteration in the level of an expression product of one or more genes selected from the group consisting of MBOAT2, PLA2G2A, PLA2G10, HPGD, GPX2, CYP4B1, CYP24A1, CYP3A5, ADH1C, ADH6, ADH7, FMO5, AKR1C1, AKR1C2, ALDH3A1, UGT1A6 and ABCC3 or of a metabolite related to one or more of said genes is indicative of cancer in said subject.

In some embodiments, if a mutation in the TP53 gene is present, an increase in the level of an expression product of MBOAT2 or of a metabolite related to MBOAT2 is indicative of cancer in said subject.

In some embodiments, if a mutation in the TP53 gene is present, a decrease in the level of an expression product of one or more genes selected from the group consisting of PLA2G2A, PLA2G10, HPGD, GPX2, CYP4B1, CYP24A1, CYP3A5, ADH1C, ADH6, ADH7, FMO5, AKR1C1, AKR1C2, ALDH3A1, UGT1A6 and ABCC3 or of a metabolite related to one or more of said genes is indicative of cancer in said subject.

In some embodiments, preferred AraX genes are those whose expression products (or related metabolites) have an altered level that is independently associated with a mutation in more than one gene (e.g. at least 2, at least 3, at least 4, at least 5 or at least 6 genes) selected from the group consisting of CTNNB1, IDH1, KEAP1, NFE2L2, NSD1, PTEN, RB1, STK11 and TP53. The identity of such preferred AraX genes can be readily derived from Table B herein.

The “increase” in the level or “increased” level of one or more of the expression products or metabolites as described herein includes any measurable increase or elevation of the expression product or metabolite in question when the expression product or metabolite in question is compared with a control level. Preferably, the level is significantly increased, compared to the level found in an appropriate control sample or subject. More preferably, the significantly increased levels are statistically significant, preferably with a probability value of <0.05. Viewed alternatively, an increase in level of the expression product or metabolite of ≧2%, ≧3%, ≧5%, ≧10%, ≧25%, ≧50%, ≧75%, ≧100%, ≧200%, ≧300%, ≧500%, ≧600%, ≧700%, ≧800%, ≧900%, ≧1000%, ≧2000%, ≧5000%, or ≧10,000% compared to the level found in an appropriate control sample or subject or population (i.e. when compared to a control level) is indicative of the presence of cancer.

The “decrease” in the level or “decreased” level of one or more of the expression products or metabolites as described herein includes any measurable decrease or reduction of the expression products or metabolites in question when the polypeptide in question is compared with a control level. Preferably, the level is significantly decreased, compared to the level found in an appropriate control sample or subject. More preferably, the significantly decreased levels are statistically significant, preferably with a probability value of <0.05. Viewed alternatively, a decrease in level of the expression product or metabolite of ≧2%, ≧3%, ≧5%, ≧10%, ≧25%, ≧50%, ≧75%, ≧100%, ≧200%, ≧300%, ≧500%, ≧600%, ≧700%, ≧800%, ≧900%, ≧1000%, ≧2000%, ≧5000%, or ≧10,000% compared to the level found in an appropriate control sample or subject or population (i.e. when compared to a control level) is indicative of the presence of cancer.

In some embodiments, an “alteration” or “altered level” of one or more expression products or metabolites is an at least 0.5 log₂ fold alteration (change) (increase or decrease) in comparison with a control level. Preferably, the alteration is at least a 0.6 log₂ fold, at least a 0.7 log₂ fold, at least a 0.8 log₂ fold, at least a 0.9 log₂ fold, at least a 1 log₂ fold, at least a 1.1 log₂ fold, at least a 1.2 log₂ fold, at least a 1.3 log₂ fold, at least a 1.4 log₂ fold, at least a 1.5 log₂ fold, at least a 1.6 log₂ fold, at least a 1.7 log₂ fold, at least a 1.8 log₂ fold, at least a 1.9 log₂ fold, at least a 2 log₂ fold, at least a 2.1 log₂ fold, at least a 2.2 log₂ fold, at least a 2.3 log₂ fold, at least a 2.4 log₂ fold, at least a 2.5 log₂ fold, at least a 2.6 log₂ fold, at least a 2.7 log₂ fold, at least a 2.8 log₂ fold, at least a 2.9 log₂ fold, at least a 3 log₂ fold, at least a 3.1 log₂ fold, at least a 3.2 log₂ fold, at least a 3.3 log₂ fold, at least a 3.4 log₂ fold, at least a 3.5 log₂ fold, at least a 3.6 log₂ fold, at least a 3.7 log₂ fold, at least a 3.8 log₂ fold, at least a 3.9 log₂ fold, at least a 4 log₂ fold, or at least a 5 log₂ fold, change (increase or decrease) in comparison with a control level. Further exemplary fold changes (increases or decreases) in relation to the expression products of the AraX network are shown in Table B herein.

In some embodiments, the level of an expression product (or related metabolite) of a single AraX gene is determined. In other embodiments, the level of an expression product (or related metabolite) of more than one AraX gene is determined (e.g. the level of expression product (or related metabolite) of two or more, or three or more, or four or more AraX genes is determined). By “more than one” is meant 2, 3, 4, 5, 6, 7, 8, 9, 10 etc. . . . 84 (including all integers between 2 and 84). A determination of the expression product (or related metabolite) level for each and every possible combination of AraX genes can be performed.

Thus, in some embodiments multi-marker methods are performed. Determining the level of expression products (or metabolites) (multiplexing) of multiple AraX genes may improve screening (e.g. diagnostic) accuracy.

In a preferred embodiment, the level of an expression product (or related metabolite) of two of the AraX genes is determined.

In a preferred embodiment, the level of an expression product (or related metabolite) of three of the AraX genes is determined.

In a preferred embodiment, the level of an expression product (or related metabolite) of four of the AraX genes is determined.

In a preferred embodiment, the level of an expression product (or related metabolite) of five of the AraX genes is determined.

In a preferred embodiment, the level of an expression product (or related metabolite) of six of the AraX genes is determined.

In a preferred embodiment, the level of an expression product (or related metabolite) of seven of the AraX genes is determined.

In some embodiments, the level of an expression product (or related metabolite) of at least 10, at least 20, at least 30, at least 40, at least 50, at least 60, at least 70 or at least 80 of the AraX genes is determined.

In one embodiment, the level of an expression product (or related metabolite) of all 84 of the AraX genes is determined.

In one embodiment, the level of an expression product (or related metabolite) of ADH1C and GPX3 are determined.

In one embodiment, the level of an expression product (or related metabolite) of ADH1C and GPX3 and CDO1 are determined.

In another embodiment, the method comprises determining the level of ADH1C in combination with (i.e. and) determining the level of at least one (e.g. 1, 2 or 3 or more) expression products (or metabolites) of the other AraX genes.

In another embodiment, the method comprises determining the level of GPX3 in combination with (i.e. and) determining the level of at least one (e.g. 1, 2 or 3 or more) expression products (or metabolites) of the other AraX genes.

In another embodiment, the method comprises determining the level of CDO1 in combination with (i.e. and) determining the level of at least one (e.g. 1, 2 or 3 or more) expression products (or metabolites) of the other AraX genes.

In some embodiments of the methods of the present invention, based on the observed alterations in the level of an expression product (or related metabolite) of one or more of the AraX genes in cancer patients (or patients suspected of having cancer) versus a control level, if desired, scoring methods, scoring systems or formulae can be employed which use such levels in order to arrive at an indication, e.g. in the form of a value or score, which can then be used for, for example, screening for, monitoring treatment of, diagnosing or prognosing cancer.

In accordance with the present invention, a score for a subject (or sample obtained from a subject) may be generated which reflects the degree of deregulation of the AraX network (AraX pathway) in comparison to the AraX network in a control subject (control sample). By deregulation of the AraX network is meant the extent/degree to which the level of one or more AraX expression products (or related metabolites) deviates from a control level. Such a score may be referred to as an AraX deregulation score. An AraX deregulation score that is altered in a test subject (or test sample) in comparison to a control may be indicative of cancer in the subject. In some embodiments, the higher the difference between the score (AraX deregulation score) for the test subject (test sample) and the control score, the higher the likelihood of cancer in the subject.

In some embodiments, the control score may be set as zero (0) and the maximum possible AraX deregulation score is one (1). In some such embodiments, the closer the score is to one, the higher the likelihood of cancer in the subject and/or the worse the prognosis.

In some embodiments, the higher the score the worse the survival prospects (e.g. 5 year survival prospects) for the subject.

In other embodiments, a scoring system/method could be designed in which a low score gives rise to a positive indication of cancer (e.g. a positive diagnosis).

Appropriate thresholds or cut-off scores/values (used to declare a sample positive or negative or to act as an indicator of prognosis/survival prospects) can be readily set by a person skilled in the art.

In some embodiments, where a control score is set as zero (0), any deviation from zero in the score for the test sample may be indicative of cancer. Preferred degrees of alteration (increases or decreases) in scores are discussed elsewhere herein in connection with altered (e.g. increased or decreased) levels.

In some embodiments, in a scoring system in which the control score is zero (0) and the maximum possible score (AraX deregulation score) is one (1), an AraX deregulation score in a test subject (test sample) of at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least, 0.75, at least 0.8, at least 0.85, at least 0.9, at least 0.95 or 1 is indicative of cancer in a subject. Preferably, an AraX deregulation score of at least 0.25 is indicative of cancer in a subject.

In some embodiments, an AraX deregulation score that is at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least, 0.75, at least 0.8, at least 0.85, at least 0.9, at least 0.95 or 1 units of score higher than the control score is indicative of cancer in a subject. Preferably, an AraX deregulation score that is at least 0.25 units of score higher than the control score is indicative of cancer in a subject.

A person skilled in the art will be familiar with suitable scoring systems and methods and any of these may be employed in connection with the present invention.

In a preferred embodiment, a score (AraX deregulation score) for the AraX pathway (or components thereof) in a sample (e.g. a tumour sample) may be obtained using Pathifier (Drier et al., 2013). This score captures the extent to which the expression of the pathway (or components thereof) in the sample deviates from its expression in the normal tissue of origin. Pathifier assigns to each sample a score which estimates the extent to which the behaviour of the pathway deviates in the sample from normal. As described in Drier et al. (2013), Pathifier analyzes NP pathways, one at a time, and assigns to each sample i and pathway P a score DP(i), which estimates the extent to which the behavior of pathway P deviates, in sample i, from normal. To determine this pathway deregulation score (PDS), the expression levels of those dP genes that belong to P are used, for example, using databases as described in Drier et al. (2013). Each sample i is a point in this dP dimensional space; the entire set of samples forms a cloud of points, the (nonlinear) “principal curve” is calculated that captures the variation of this cloud. Next, each sample is projected onto this curve; the PDS is defined as the distance DP(i), measured along the curve, of the projection of sample i, from the projection of the normal samples.

In other embodiments, AraX deregulation is calculated with other standard correlation metrics, such as Pearson correlation or Spearman correlation.

As discussed above, the present invention provides a method for screening for cancer in a subject. Alternatively viewed, the present invention provides a method of diagnosing cancer in a subject. Alternatively viewed, the present invention provides a method for the prognosis of cancer in a subject (prognosis of the future severity, course and/or outcome of cancer). Alternatively viewed, the present invention provides a method of determining the clinical severity of cancer in a subject. Alternatively viewed, the present invention provides a method for predicting and monitoring the response of a subject to therapy. Alternatively viewed, the present invention provides a method for detecting the recurrence of cancer. Alternatively viewed, the present invention provides a method for determining the aggressiveness of cancer, e.g. distinguishing between indolent and aggressive cancer (and thus may e.g. inform a decision between active surveillance and treatment). Alternatively viewed, the present invention provides a method for predicting the survival prospects for a cancer patient (e.g. 5 year survival prospects).

Thus, the method of screening for cancer in accordance with the present invention can be used, for example, for diagnosing cancer, for the prognosis of cancer, for monitoring the progression of cancer, for determining the clinical severity of cancer, for predicting and monitoring the response of a subject to therapy, for determining the efficacy of a therapeutic regime being used to treat cancer, for detecting the recurrence of cancer, for distinguishing between indolent and aggressive cancer, or for predicting the survival prospects for a cancer patient.

Thus, in one aspect the present invention provides a method for diagnosing cancer in a subject. In some embodiments, a positive diagnosis is made (i.e. a diagnosis of the presence of cancer) if the level of an expression product (or related metabolite) of one or more of the AraX genes in the sample is altered (increased or decreased) in comparison to a control level. Expression products (or related metabolites) for which an altered level is indicative of (e.g. diagnostic of) cancer are described elsewhere herein.

In another aspect, the present invention provides a method for selecting patients suspected of having cancer for further diagnosis. In some embodiments, a positive indication is made if the level of an expression product (or related metabolite) of one or more of the AraX genes in the sample is altered (increased or decreased) in comparison to a control level.

In another aspect, the present invention provides a method for the prognosis of cancer in a subject. In such methods the level of an expression product (or related metabolite) of one or more of the AraX genes discussed above in the sample is indicative of the future severity, course and/or outcome of cancer. For example, an alteration (increase or decrease) in the level of an expression product (or related metabolite) of one or more of the AraX genes in the sample in comparison to a control level may indicate a poor prognosis. A highly altered level may indicate a particularly poor prognosis.

In some embodiments, an increased level of an expression product (or related metabolite) of one or more of the AraX genes is suggestive of (i.e. indicative of) a poor prognosis. In some embodiments, a decreased level of an expression product (or related metabolite) of one or more of the AraX genes is suggestive of (i.e. indicative of) a poor prognosis. Examples of appropriate expression products (or related metabolites) of AraX genes which can be increased or decreased are provided elsewhere herein.

In some embodiments, for example, the more altered the level or score (e.g. in comparison to a control level or score), the greater the likelihood of a poor (or worse) prognosis. In some embodiments, for example, the less altered the level or score (e.g. in comparison to a control level of score), the greater the likelihood of a good prognosis. In some such embodiments of prognostic methods of the invention, a good (or better) prognosis may be a good (or better) prognosis relative to the prognosis for a control or reference subject or control or reference population with a known outcome or a known probability of outcome (e.g. average (e.g. median) prognosis (e.g. survival) for a control population). In some such embodiments of prognostic methods of the invention, a poor prognosis may be poor (or worse) prognosis relative to the prognosis for a control or reference subject or control or reference population with a known outcome or a known probability of outcome (e.g. average (e.g. median) prognosis (e.g. survival) for a control population).

Serial (periodic) measuring of the level of an expression product (or related metabolite) of one or more of the AraX genes may also be used for prognostic purposes looking for either increasing or decreasing levels over time. In some embodiments, an altering level (increase or decrease) of an expression product (or related metabolite) of one or more of the AraX genes over time (in comparison to a control level) may indicate a worsening prognosis. In some embodiments, an altering level (increase or decrease) of an expression product (or related metabolite) of one or more of the AraX genes over time (in comparison to a control level) may indicate an improving prognosis. Thus, the methods of the present invention can be used to monitor disease progression. Such monitoring can take place before, during or after treatment of cancer by surgery or therapy. Thus, in one aspect the present invention provides a method for monitoring the progression of cancer in a subject.

Methods of the present invention can be used in the active monitoring of patients which have not been subjected to surgery or therapy, e.g. to monitor the progression of cancer in untreated patients. Again, serial measurements can allow an assessment of whether or not, or the extent to which, the cancer is worsening, thus, for example, allowing a more reasoned decision to be made as to whether therapeutic intervention is necessary or advisable.

Monitoring can also be carried out, for example, in an individual who is thought to be at risk of developing cancer, in order to obtain an early, and ideally pre-clinical, indication of cancer. In this way, it can be seen that in some embodiments of the invention, the methods can be carried out on “healthy” patients (subjects) or at least patients (subjects) which are not manifesting any clinical symptoms of cancer, for example, patients with very early or pre-clinical stage cancer, e.g. patients where the primary tumor is so small that it cannot be assessed or detected or patients in which cells are undergoing pre-cancerous changes associated with cancer but have not yet become malignant.

In another aspect, the present invention provides a method for determining the clinical severity of cancer in a subject. In such methods the level of an expression product (or related metabolite) of one or more of the AraX genes in the sample shows an association with the severity of the cancer. Thus, the level of an expression product (or related metabolite) of one or more of the AraX genes is indicative of the severity of the cancer. In some embodiments, the more altered (more increased or more decreased as the case may be) the level of an expression product (or related metabolite) of one or more of the AraX genes in comparison to a control level, the greater the likelihood of a more severe form of cancer (e.g. the greater the likelihood of a more aggressive form of cancer). In some embodiments the methods of the invention can thus be used in the selection of patients for therapy.

Serial (periodical) measuring of the level of an expression product (or related metabolite) of one or more of the AraX genes may also be used to monitor the severity of cancer looking for altering (e.g. increasing or decreasing) levels over time. Observation of altered levels (increase or decrease as the case may be) may also be used to guide and monitor therapy, both in the setting of subclinical disease, i.e. in the situation of “watchful waiting” (also known as “active surveillance”) before treatment or surgery, e.g. before initiation of pharmaceutical therapy, or during or after treatment to evaluate the effect of treatment and to look for signs of therapy failure.

The present invention also provides a method for predicting the response of a subject to therapy. In such methods the choice of therapy may be guided by knowledge of the level in the sample of an expression product (or related metabolite) of one or more of the AraX genes.

The present invention also provides a method of determining (or monitoring) the efficacy of a therapeutic regime being used to treat cancer. In such methods, an alteration (increase or decrease) in the level of an expression product (or related metabolite) of one or more of the AraX genes indicates the efficacy of the therapeutic regime being used. For example, if the level of an expression product (or related metabolite) of one or more of the AraX genes moves towards the control level during (or after) therapy, this is indicative of an effective therapeutic regime. In such methods, serial (periodical) measuring of the level of an expression product (or related metabolite) of one or more of the AraX genes over time can also be used to determine the efficacy of a therapeutic regime being used.

The present invention also provides a method for detecting the recurrence of cancer.

Alternatively viewed, the present invention provides a method of screening for cancer in a subject, said method comprising determining whether or not (or the extent to which) the AraX network (which comprises the 84 AraX genes described elsewhere herein) is deregulated in comparison with a control, wherein deregulation of the AraX network in comparison with a control is indicative of cancer. Whether or not (or the extent to which) the AraX network is deregulated may be determined by determining the level in a sample of an expression product of one or more of the AraX genes (or of a related metabolite), wherein said sample has been obtained from a subject and wherein an altered level in said sample of the expression product (or related metabolite) of one or more AraX genes in comparison to a control level indicates deregulation of the AraX network and is indicative of cancer in said subject.

The features and discussion herein in relation to the method of screening for cancer (e.g. in relation to preferred AraX genes or combinations thereof discussed above) apply, mutatis mutandis, to the other related methods of present invention (e.g. to a method of diagnosing cancer etc.).

In one embodiment, the invention provides the use of the methods (e.g. screening, diagnostic or prognostic methods) in conjunction other known screening, diagnostic or prognostic methods. Thus, for example, the methods of the invention can be used to confirm a diagnosis of cancer in a subject. In some embodiments the methods of the present invention are used alone.

In some aspects, methods of the invention are provided which further comprise a step of treating cancer by therapy (e.g. pharmaceutical therapy such as chemotherapy) or surgery (e.g. removal of the cancerous/tumour tissue). For example, if the result of the method of the invention is indicative of cancer in the subject (e.g. a positive diagnosis of cancer is made), then an additional step of treating cancer by therapy or surgery can be performed. Methods of treating cancer by therapy or surgery are known in the art.

In some embodiments, methods of the invention (e.g. screening or diagnosis methods) which further comprise a step of treating cancer may comprise administering to the subject a therapeutically effective amount of one or more agents selected from the group consisting of Lenalidomide, Trabectedin, Etoposide, Aldesleukin, Imatinib, Resveratrol, Cisplatin, Masoprocol, Bestatin, Ethyl carbamate, Epirubicin, Suramin, Quinacrine, Aldesleukin, Canfosfamide, Carmustine, Busulfan, Oxaliplatin, Chlorambucil, Azathioprine and Carboplatin, or one or more agents (drugs) that targets cytochrome p450 metabolism.

In some embodiments, if the level of an expression product(s) or related metabolite(s) (or the deregulation score) is altered by a particular degree in comparison to a control level or score then a further step of administering a therapeutically effective amount of a pharmaceutical agent (e.g. a chemotherapeutic agent) to the patient is performed and/or surgery is performed. Preferred degrees of alteration are discussed elsewhere herein. In some embodiments, if a subject is already undergoing pharmaceutical therapy (e.g. chemotherapeutic therapy) and the level of an expression product(s) or related metabolite(s) (or deregulation score) is altered by a particular degree in comparison to a control level (e.g. in comparison to a previously recorded level or score for the same subject) then this may be indicative that a therapeutic agent other that the previous therapeutic agent should be used and thus a step of administering a therapeutically effective amount of a therapeutic agent (e.g. a chemotherapruic agent) other than the therapeutic agent (e.g. chemotherapeutic agent) previously administered to the subject may be performed. In some embodiments, if a method of the invention reveals that a current treatment regimen is ineffective (e.g. if serial or periodic measurements of expression product (or related metabolite) levels or scores in the subject reveal treatment is being ineffective), a step of altering (e.g. increasing) the dosage of the therapeutic agent may be performed.

In another aspect, the present invention provides a method for treating cancer, which method comprises administering to a subject in need thereof a therapeutically effective amount of an agent which modulates the level and/or activity of one or more components (e.g. expression products) of the AraX network. Such methods of treating cancer may involve administering to a subject in need thereof a therapeutically effective amount of an agent that restores the level of an expression product (or related metabolite) of one or more of the AraX genes to (or close to) a control level. In some embodiments, more than one (e.g. 2, 3, 4, 5, 6 etc.) different agents may be used, each targeting different AraX components (e.g. expression products). Thus, in some embodiments a multi-target approach may be taken.

In some embodiments, one or more metabolites related to expression products of the AraX network are targeted.

A therapeutically effective amount can be determined based on the clinical assessment and can be readily monitored.

Modulation (alteration) of level or activity may be an increase or decrease of level or activity. For example, if an expression product of an AraX gene has a lower level or activity in a cancer sample than a control level, then the agent may increase the level or activity of the expression product to restore the level or activity of the expression product to (or close to), or significantly towards the control level. Conversely, if an expression product of an AraX gene has a higher level or activity in a cancer sample than a control level, then the agent may decrease the level or activity of the expression product to restore the level or activity of the expression product to (or close to), or significantly towards the control level.

Any agent which modulates the level and/or activity of one or more components of the AraX network may be used in accordance with the present invention. Modulatory molecules (agents) may act at the nucleic acid level, for example they may increase or decrease, as the case may be, the expression of an AraX gene, thereby, for example, resulting in increased or decreased mRNA level of an AraX gene. Preferably, the modulation (increase or decrease) is a significant modulation (alteration), more preferably a statistically significant modulation, preferably with a probability value of 0.05. In some embodiments, the agents achieve a modulation (increase or decrease as the case may be) of at least 10%, at least 20%, at least 30%, at least 40%, at least 50%, at least 60%, at least 70%, at least 80%, at least 90%, or at least 100% of AraX gene mRNA levels.

Modulatory molecules may alternatively, or in addition, act at the level of the protein and inhibit or increase the functional activity of, for example, a protein (e.g. an enzyme) encoded by an AraX gene. Modulation at the protein level may be, for example, by reducing the level (and/or by altering post-translational modifications) of the protein encoded by an AraX gene thereby reducing functional activity, and/or by directly inhibiting (reducing) the functional activity of the protein encoded by an AraX gene by, for example, binding to the protein as an active site inhibitor or as an exosite inhibitor such that functional activity is reduced. Conversely, modulation at the protein level may be, for example, by increasing the level (and/or by altering post-translational modifications) of the protein encoded by an AraX gene thereby increasing functional activity, and/or by directly increasing the functional activity of the protein encoded by an AraX gene by, for example, binding to the protein such that functional activity is increased. Preferably, the modulation (increase or decrease) is a significant modulation, more preferably a statistically significant modulation, preferably with a probability value of 0.05. In some embodiments, the modulatory molecules achieve a modulation (increase or decrease) of at least 10%, at least 20%, at least 30%, at least 40%, at least 50%, at least 60%, at least 70%, at least 80%, at least 90%, or at least 100% in the level or functional activity of the protein encoded by an AraX gene.

The references to “reduce”, “reducing”, “reduction”, “decrease”, “increase”, “modulation” or “alteration” in the above discussions of expression and functional activity mean in comparison to in the absence of the modulatory molecule (agent).

Suitable agents which modulate the activity of one or more components of the AraX network may be described in the art. Alternatively, suitable agents can be readily screened for and identified by a person skilled in the art using assays that are routine in the art. By way of example, such a method for identifying an agent which modulates the level or activity of one or more components of the AraX network may comprise: (1) contacting a preparation with a test agent (i.e. a candidate agent), wherein the preparation comprises (i) a protein (e.g. an enzyme) encoded by an AraX gene, or at least a biologically active fragment thereof and optionally a substrate for said protein; or (ii) a polynucleotide comprising at least a portion of a genetic sequence that regulates the expression of an AraX gene (e.g. a promoter and/or an enhancer of an AraX gene), which is operably linked to a reporter gene; and (2) detecting a change in the level and/or functional activity of the protein (e.g. an enzyme) encoded by an AraX gene, or the level of expression of the reporter gene. Such a level and/or functional activity can be compared to a normal or reference (control) level and/or functional activity in the absence of the test agent. An alteration (increase or decrease) in the level and/or activity of the protein or an alteration (increase or decrease) in the level of expression of the reporter gene would indicate that the test agent is an agent that modulates a component of the AraX network.

mRNA levels of (i.e. transcribed from) an AraX gene in a cell or tissue after contacting with a test agent can also be monitored using standard techniques in the art (e.g. qRT-PCR). An alteration in an AraX gene's mRNA level would indicate that the test agent is an agent which modulates the level of a component of the AraX network.

The screening assays disclosed herein may be performed in conventional or high-throughput formats.

The sources for potential agents to be screened include natural sources, such as a cell extracts, and synthetic sources such as chemical compound libraries, or biological libraries such as antibody or peptide libraries or siRNA libraries. Sources for potential agents also include gene editing libraries (e.g. based on CRISPR-Cas9 technologies) or gene interfering libraries (e.g. siRNA libraries).

Agents (modulatory molecules) that may be used in accordance with the present invention include, but are not limited to, antisense DNA or RNA molecules, RNAi molecules, ribozymes, shRNA molecules, siRNA molecules, miRNA molecules, small regulatory RNA molecules, non coding RNAs and the like which are directed against an AraX gene (or a transcript of an AraX gene). Once an AraX gene target for inhibition has been selected, it is routine in the art to design and synthesise such nucleic acid based inhibitory molecules, based on the nucleic acid sequence of the inhibitor's target. Nucleic acid sequences of AraX genes are known in the art.

Other agents that may be used in accordance with the present invention are antibodies, or antigen-binding fragments thereof, which bind to a protein encoded by an AraX gene. It is well known in the art that upon binding of an antibody to its protein (antigen) target, the function of that protein target may be inhibited. Likewise, that antibodies may act as agonists of their protein target is also well-known in the art. Once a protein encoded by an AraX gene has been selected, standard techniques in the art (e.g. phage display) can be used to generate antibodies against that enzyme and routine tests can be performed to subsequently identify antibodies with inhibitory or agonistic activity.

Gene therapy may also be used in connection with the methods of treating cancer of the present invention. For example, genome editing (also know as genome editing with engineered nucleases) techniques may be used, e.g. the CRISPR-Cas9 system. In accordance with the present invention DNA (coding DNA or non-coding DNA) may be delivered into cells, for example by methods using recombinant viruses or naked DNA/DNA complexes.

In some embodiments, the agent is not aspirin.

In some embodiments of the methods for treating cancer, the agent is not Lenalidomide, Trabectedin, Etoposide, Aldesleukin, Imatinib, Resveratrol, Cisplatin, Masoprocol, Bestatin, Ethyl carbamate, Epirubicin, Suramin, Quinacrine, Aldesleukin, Canfosfamide, Carmustine, Busulfan, Oxaliplatin, Chlorambucil, Azathioprine or Carboplatin.

In some embodiments of the methods for treating cancer, the agent is not a drug that targets cytochrome p450 metabolism.

In some embodiments of the method of treating cancer in accordance with the invention, one or more of the following AraX genes are not targeted: PLA2G2A, PLA2G4A, PTGS2, GSR, NQO2. In some embodiments of the method of treating cancer in accordance with the invention, ADH7 is not targeted.

In some embodiments of the method of treating cancer in accordance with the invention, said method comprises administering to a subject in need thereof a therapeutically effective amount of an agent which modulates the level and/or activity of an expression product, or related metabolite, of one or more genes selected from the group consisting of ADH7, GSTM3, ABCC2, PTGR1 and CBR3.

In another aspect, the present invention provides a method for treating cancer, which method comprises administering to a subject in need thereof a therapeutically effective amount of an agent which modulates the Keapl-Nrf3 pathway. The Keap1-Nrf3 pathway is the major regulatory axis associated with the AraX pathway (AraX network). The features and discussion herein in relation to the method of treatment of cancer by modulating AraX genes apply, mutatis mutandis, to this aspect of the invention.

Agents for use in treating cancer in accordance with the methods of the present invention may be included in formulations. Such formulations may be for pharmaceutical or veterinary use. Suitable diluents, excipients and carriers for use in such formulations are known to the skilled man.

The compositions (formulations) may be presented, for example, in a form suitable for oral, nasal, parenteral, intravenal, topical or rectal administration, preferably in a form suitable for oral administration.

The active compounds (agents) defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions are then filled into injection vials or ampoules.

Nasal sprays may be formulated similarly in aqueous solution and packed into spray containers, either with an aerosol propellant or provided with means for manual compression.

The pharmaceutical compositions (formulations) may be administered parenterally. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the active compound in the form of a nasal or pulmonal spray. As a still further option, the active compound can also be administered transdermally, e.g. from a patch, optionally a iontophoretic patch, or transmucosally, e.g. bucally.

Dosages may vary based on parameters such as the age, weight and sex of the subject. Appropriate dosages can be readily established. Appropriate dosage units can readily be prepared.

The pharmaceutical compositions may additionally comprise further active ingredients.

In preferred embodiments, the level in a sample of an expression product of one or more AraX genes is determined.

As referred to herein, an “expression product” of a gene includes mRNA molecules transcribed from the gene or polypeptides (proteins, e.g. enzymes) encoded by the gene. The level of the mRNA or polypeptide (protein) in question can be determined by analysing the sample which has been obtained from or removed from the subject by an appropriate means. The determination is typically carried out in vitro.

Nucleotide and amino acid sequences of the genes of the AraX network (and of other genes mentioned herein) are known in the art, for example such sequences are provided in the Uniprot database (see e.g. Table 1 and Table 2 herein) (http://www.uniprot.org/)

It will be appreciated that an mRNA molecule will comprise the same sequence as the DNA molecule from which it was transcribed, with the exception the mRNA molecule will comprise uracil whereas the DNA molecule from which it was transcribed would instead comprise thymine at the corresponding positions.

In one embodiment, the expression product detected by the methods of the invention is an mRNA molecule. As discussed elsewhere herein, it is not necessary to detect the presence of the entire mRNA molecule (i.e. the entire mRNA nucleotide sequence); detecting the presence of a fragment or portion of an mRNA molecule can be indicative of the presence of the entire mRNA molecule. In some embodiments, methods may comprise determining the presence or level of a non-naturally occurring fragment or portion of an mRNA molecule.

In another embodiment, the expression product detected by the methods of the invention is a polypeptide. As discussed elsewhere herein, it is not necessary to detect the presence of the entire polypeptide (i.e. the polypeptide's entire amino acid sequence); detecting the presence of a fragment or portion of a polypeptide may be indicative of the presence of the entire polypeptide. In some embodiments, methods may comprise determining the presence or level of a non-naturally occurring fragment or portion of a polypeptide.

A number of different methods for detecting nucleic acids (e.g. mRNA) are known and described in the literature and any of these may be used according to the present invention. At its simplest, the nucleic acid may be detected by hybridisation to a probe (e.g. an oligonucleotide probe) and many such hybridisation protocols have been described (see e.g. Sambrook et al., Molecular cloning: A Laboratory Manual, 3rd Ed., 2001, Cold Spring Harbor Press, Cold Spring Harbor, N.Y.). Typically, the detection will involve a hybridisation step and/or an in vitro amplification step.

In one embodiment, the target nucleic acid in a sample may be detected by using an oligonucleotide with a label attached thereto, which can hybridise to the nucleic acid sequence of interest. Such a labelled oligonucleotide will allow detection by direct means or indirect means. In other words, such an oligonucleotide may be used simply as a conventional oligonucleotide probe. After contact of such a probe with the sample under conditions which allow hybridisation, and typically following a step (or steps) to remove unbound labelled oligonucleotide and/or non-specifically bound oligonucleotide, the signal from the label of the probe emanating from the sample may be detected. In preferred embodiments the label is selected such that it is detectable only when the probe is hybridised to its target.

In another embodiment, the target nucleic acid (e.g. mRNA) in a sample may be determined by using an oligonucleotide probe which is labelled only when hybridised to its target sequence, i.e. the probe may be selectively labelled. Conveniently, selective labelling may be achieved using labelled nucleotides, i.e. by incorporation into the oligonucleotide probe of a nucleotide carrying a label. In other words, selective labelling may occur by chain extension of the oligonucleotide probe using a polymerase enzyme which incorporates a labelled nucleotide, preferably a labelled dideoxynucleotide (e.g. ddATP, ddCTP, ddGTP, ddTTP, ddUTP). This approach to the detection of specific nucleotide sequences is sometimes referred to as primer extension analysis. Suitable primer extension analysis techniques are well known to the skilled man, e.g. those techniques disclosed in WO99/50448, the contents of which are incorporated herein by reference.

In one embodiment of the present invention, the presence and level of mRNA gene products, or fragments thereof, are detected by a primer-dependent nucleic acid amplification reaction. The amplification reaction is allowed to proceed for a duration (e.g. number of cycles) and under conditions that generate a sufficient amount of amplification product. Most conveniently the polymerase chain reaction (PCR) will be used, although the skilled man would be aware of other techniques. For instance LAR/LCR, SDA, Loop-mediated isothermal amplification and nucleic acid sequence based amplification (NASBA)/3SR (Self-Sustaining Sequence Replication) may be used. If an mRNA gene product is to be detected, it will generally first be converted into a cDNA molecule by reverse transcription using a reverse transcriptase enzyme to generate a cDNA molecule. Upon completion of the reverse transcription reaction, the cDNA can be used as the template for the primer-dependent nucleic acid amplification reaction. A person skilled in the art will be well aware of how to generate cDNA molecules from mRNA molecules.

Many variations of PCR have been developed, for instance Real Time PCR (also known as quantitative PCR, qPCR), hot-start PCR, competitive PCR, and so on, and these may all be employed where appropriate to the needs of the skilled man.

In one basic embodiment using a PCR based amplification, oligonucleotide primers are contacted with a reaction mixture containing or potentially containing the target sequence and free nucleotides in a suitable buffer. Thermal cycling of the resulting mixture in the presence of a DNA polymerase results in amplification of the sequence between the primers.

Optimal performance of the PCR process is influenced by choice of temperature, time at temperature, and length of time between temperatures for each step in the cycle. A person skilled in the art is readily able to do this.

Methods of the present invention may be performed with any of the standard mastermixes and enzymes available.

Modifications of the basic PCR method such as qPCR (Real Time PCR) have been developed that can provide quantitative information on the template being amplified. Numerous approaches have been taken although the two most common techniques use double-stranded DNA binding fluorescent dyes or selective fluorescent reporter probes.

Double-stranded DNA binding fluorescent dyes, for instance SYBR Green, associate with the amplification product as it is produced and when associated the dye fluoresces. Accordingly, by measuring fluorescence after every PCR cycle, the relative amount of amplification product can be monitored in real time. Through the use of internal standards and controls, this information can be translated into quantitative data on the amount of template at the start of the reaction.

Fluorescent reporter probes used in qPCR may be sequence specific oligonucleotides, typically RNA or DNA, that have a fluorescent reporter molecule at one end and a quencher molecule at the other (e.g. the reporter molecule is at the 5′ end and a quencher molecule at the 3′ end or vice versa). The probe is designed so that the reporter is quenched by the quencher. The probe is also designed to hybridise selectively to particular regions of complementary sequence which might be in the template. If these regions are between the annealed PCR primers the polymerase, if it has exonuclease activity, will degrade (depolymerise) the bound probe as it extends the nascent nucleic acid chain it is polymerising. This will relieve the quenching and fluorescence will rise. Accordingly, by measuring fluorescence after every PCR cycle, the relative amount of amplification product can be monitored in real time. Through the use of internal standard and controls, this information can be translated into quantitative data.

The amplification product may be detected, and amounts (levels) of amplification product can be determined by any convenient means. A vast number of techniques are routinely employed as standard laboratory techniques and the literature has descriptions of more specialised approaches. At its most simple the amplification product may be detected by visual inspection of the reaction mixture at the end of the reaction or at a desired time point. Typically the amplification product will be resolved with the aid of a label that may be preferentially bound to the amplification product. Typically a dye substance, e.g. a colorimetric, chromomeric fluorescent or luminescent dye (for instance ethidium bromide or SYBR green) is used. In other embodiments a labelled oligonucleotide probe that preferentially binds the amplification product is used.

In some embodiments, a microarray may be used to determine the level of nucleic acid expression products of one or more of the AraX genes.

In some embodiments, RNA-seq by next generation sequencing may be used to determine the level of nucleic acid expression products of one or more of the AraX genes. RNA-seq (RNA sequencing) is sometimes referred to as whole transcriptome shotgun sequencing (WTSS). RNA-seq uses the capabilities of next generation sequencing to reveal a snapshot of RNA presence and quantity from a genome at a given moment in time. In some cases RNA can be converted to cDNA (via reverse transcription) prior to sequencing. In other cases RNA can be directly sequenced without conversion to cDNA. In some cases, cDNA is followed by adapter ligation prior to sequencing. RNA or cDNA is subsequently amplified by PCR to generate sufficient quantities of fragments prior to sequencing. In some cases, dUTP is incorporated during second strand cDNA synthesis to prevent PCR amplification and reduce bias introduced by PCR in the level determination. In some cases, a different adapter of known orientation is incorporated during second strand cDNA synthesis.

Suitable microarray platforms or machines and suitable RNA-seq platforms or machines are known in the art and can be used in the present invention. Suitable platforms or machines include those from manufacturers including Affymetrix, Agilent, Applied Microarrays, Arrayit, Illumina, and Pacific Biosciences, for example platforms or machines such as Affymetrix GeneChip Systems, Illumina MiniSeq System, Illumina MiSeq Series, Illumina NextSeq System, Illumina HiSeq Series, Pacific Biosciences PacBio RS II, or Pacific Biosciences Sequel Systems.

In some preferred embodiments of the present invention measuring the level of one or more expression products is by a nucleic acid (DNA/RNA) based method and preferably involves nucleic acid amplification.

Levels of one or more of the polypeptides in the sample can be measured (determined) by any appropriate assay, a number of which are well known and documented in the art and some of which are commercially available. The level of one or more of the polypeptides (proteins/biomarkers) can be determined e.g. by an immunoassay such as a radioimmunoassay (RIA) or fluorescence immunoassay, immunoprecipitation and immunoblotting (e.g. Western blotting) or Enzyme-Linked ImmunoSorbent Assay (ELISA). Immunoassays are a preferred technique for determining the levels of one or more of the polypeptides in accordance with the present invention.

Preferred assays are ELISA-based assays, although RIA-based assays can also be used effectively. Both ELISA- and RIA-based methods can be carried out by methods which are standard in the art and would be well known to a skilled person. Such methods generally involve the use of an antibody to a relevant polypeptide under investigation, or fragment thereof, which is incubated with the sample to allow detection of said polypeptide (or fragment thereof) in the sample. Any appropriate antibodies can be used. For example, an appropriate antibody to a polypeptide under investigation, or an antibody which recognises particular epitopes of said polypeptide, can be prepared by standard techniques, e.g. by immunization of experimental animals, which are known to a person skilled in the art. The same antibody to a given polypeptide under investigation or fragments thereof can generally be used to detect said polypeptide in either a RIA-based assay or an ELISA-based assay, with the appropriate modifications made to the antibody in terms of labeling etc., e.g. in an ELISA assay the antibodies would generally be linked to an enzyme to enable detection. Any appropriate form of assay can be used, for example the assay may be a sandwich type assay or a competitive assay.

In simple terms, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light of the appropriate wavelength is shone upon the sample, any antigen/antibody complexes will fluoresce so that the amount of antigen in the sample can be determined through the magnitude of the fluorescence. For RIA, a known quantity of an antigen is made radioactive, frequently by labeling it with gamma-radioactive isotopes of iodine attached to tyrosine. This radiolabeled antigen is then mixed with a known amount of antibody for that antigen, and as a result, the two chemically bind to one another. Then, a sample from a patient containing an unknown quantity of that same antigen is added. This causes the unlabeled (or “cold”) antigen from the sample to compete with the radiolabeled antigen for antibody binding sites. As the concentration of “cold” antigen is increased, more of it binds to the antibody, displacing the radiolabeled variant, and reducing the ratio of antibody-bound radiolabeled antigen to free radiolabeled antigen. The bound antigens are then separated from the unbound ones, and the radioactivity of the free antigen remaining in the supernatant is measured. A binding curve can then be plotted, and the exact amount of antigen in the patient's sample can be determined. Measurements are usually also carried out on standard samples with known concentrations of marker (antigen) for comparison.

In some embodiments, immunohistochemistry with appropriate antibodies could be carried out.

The use of immunoblotting (e.g. Western blotting) can also be used for measuring the level of one or more of the polypeptides in accordance with the present invention.

Preferred agents for use in determining the level of one or more of the polypeptides in accordance with the present invention are antibodies (antibodies to the polypeptide whose level is to be determined).

In other preferred embodiments, the level of one or more of the polypeptides (or fragments thereof such as non-naturally occurring fragments) in the sample can be measured (determined) by mass spectrometry. Suitable mass spectrometry methods (and associated data processing techniques) are well known and documented in the art. In some embodiments mass spectrometry (and associated data processing techniques) is used to obtain a ratio of the level of a polypeptide (or fragment thereof) in the sample in comparison to a control. In some embodiments, protein fragments (e.g. non-naturally occurring fragments) may be quantified using chromatography coupled with mass spectrometry.

Reference herein to the “polypeptides” whose level is to be determined in accordance with the invention includes reference to all forms of said polypeptides (as appropriate) which might be present in a subject, including derivatives, mutants and analogs thereof, in particular fragments thereof (e.g. naturally occurring fragments or non-naturally occurring fragments) or modified forms of the polypeptides or their fragments. Exemplary and preferred modified forms include forms of these molecules which have been subjected to post translational modifications such as glycosylation or phosphorylation. In some embodiments, the level of unmodified forms of the polypeptides (or their fragments) is determined.

It is well understood in the art that when detecting the presence of a polypeptide (protein) in a sample, it is not necessary to detect the presence of the full-length polypeptide (i.e. the entire polypeptide sequence); detecting the presence of a fragment (or portion) of a polypeptide can be indicative of the presence of the entire polypeptide (protein).

Thus, in certain embodiments of the methods of the invention described herein, any fragments (or portions) of the polypeptides, in particular naturally occurring fragments, can be analysed as an alternative to the polypeptides themselves (full length polypeptides). Suitable fragments for analysis should be characteristic of the full-length polypeptide (protein). Suitable fragments can be at least 6 consecutive amino acids in length. For example, at least 7, at least 8, at least 9, at least 10, at least 15, at least 20, at least 25, at least 50, at least 75, at least 100, at least 150, at least 200 or at least 500 consecutive amino acids in length. Suitable fragments can represent at least 5%, at least 10%, at least 20%, at least 30%, at least 40%, at least 50%, at least 60%, at least 70%, at least 80%, or at least 90% of the length of the full-length polypeptide (protein).

In some embodiments the level of the full-length polypeptide is determined.

It is also well understood in the art that when detecting the presence of an mRNA in a sample it is not necessary to detect the presence of the entire mRNA molecule (i.e. the entire mRNA nucleotide sequence); detecting the presence of a fragment (or portion) of an mRNA molecule can be indicative of the presence of the entire mRNA molecule.

Thus, in certain embodiments of the methods of the invention described herein, any fragments (or portions) of the mRNAs can be analysed as an alternative to the full length mRNAs. Suitable fragments for analysis should be characteristic of the full-length mRNA.

Suitable fragments can be at least 17 nucleotides in length. For example, at least 18, at least 19, at least 20, at least 25, at least 30, at least 35, at least 40, at least 50, at least 75, at least 100, at least 150, at least 200 or at least 500 consecutive nucleotides in length. Suitable fragments can represent at least 5%, at least 10%, at least 20%, at least 30%, at least 40%, at least 50%, at least 60%, at least 70%, at least 80%, or at least 90% of the length of the full-length mRNA molecule.

In some embodiments the level of the full-length mRNA molecule is determined.

In some embodiments, the level in a sample of a metabolite related to (or associated with) an expression product of one or more of the AraX genes is determined. Put another way, the level in a sample of a metabolite of an expression product of one or more of the AraX genes is determined.

Metabolites are generally small molecules. Metabolites can be the ultimate effectors of expression products. Thus, metabolites related to (e.g. produced by) expression products (e.g. polypeptides/enzymes) of one or more AraX genes may have their level determined in accordance with the present invention. The level of one or more metabolites may be determined in addition to, or as an alternative to, determining the level of expression products themselves. An altered level of a metabolite(s) related to an expression product of one or more AraX genes in comparison to a control level is indicative of cancer in a subject. Exemplary degrees of alteration (e.g. increases or decreases) are discussed elsewhere herein.

Exemplary and preferred metabolites are described in the Example section.

In some embodiments, the metabolite is a metabolite of the cytochrome P450 pathway.

In some embodiments, the metabolite is a metabolite of the hydroxylase pathway.

In some embodiments, the metabolite is a metabolite of the epoxygenase pathway.

In some embodiments, the metabolite is a metabolite of the cyclooxygenase pathway. The cyclooxygenase pathway produces prostaglandins. Thus, in some embodiments, the metabolite is a prostaglandin. Preferred prostaglandins are prostaglandin H₂, prostaglandin E₂, prostaglandin F₂₋ₐₗₚₕₐ, prostaglandin D₂, and 11-beta-prostaglandin F₂₋ₐₗₚₕₐ,

In some embodiments, the metabolite is a metabolite of the lipoxygenase pathway. In some embodiments the metabolite is a leukotriene. Preferred leukotrienes are leukotriene A₄, leukotriene B₄, leukotriene C₄, and leukotriene D₄.

In some embodiments, the metabolite is a lipoxilin.

In some embodiments, the metabolite is cannabinoid anandamide.

In some embodiments, the metabolite is cysteine or tyrosine.

In some embodiments, the metabolite is an iron ion.

In some embodiments, preferred metabolites are arachidonic acid or glutathione.

In some embodiments, the level of a metabolite may be determined using gas/liquid chromatography coupled with mass-spectrometry.

Suitable gas/liquid chromatography platforms or machines and mass-spectrometry platforms or machines are known in the art and can be used in the present invention. Suitable platforms or machines include those from manufacturers including AB Sciex, Agilent, Applied Biosystems, Bruker, GenTech Scientific, Hitachi High Technologies, IONICON, JEOL, LECO, PerkinElmer, Shimadzu, Thermo Fisher Scientific, or Waters.

In some embodiments, the level of a metabolite may be determined using assay kits.

Suitable kits are known in the art and can be used in the present invention. Suitable kits include those from manufacturers including Roche, Sigma-Aldrich, or Thermo Fisher Scientific.

In some embodiments, the level of an expression product (or related metabolite) in association with (e.g. physical association with or in complex with) the reagent that is being used to detect the expression product (or related metabolite) is determined. Thus, in some embodiments the level of a complex of an expression product (or related metabolite) and the reagent used to detect the expression product (or related metabolite) is determined. Reagents suitable for detecting expression products (or related metabolites) are discussed elsewhere herein. Purely by way of example, in some embodiments the level of a nucleic acid (DNA or RNA) expression product in association with (e.g. in complex with) a primer (or extended primer) or probe (e.g fluorecent reporter probe) or dye or the like may be determined. By way of another example, in some embodiments the level of a polypeptide expression product in association with (e.g. in complex with) an antibody may be determined. By way of another example, in some embodiments the level of a related metabolite in association with (e.g. in complex with) a chemical derivative (e.g. a silyl group for example with general formula R₃Si or an alkyl group for example with general formula CₙH₂ₙ₊₁) may be determined.

A “control level” is the level of an expression product or of a related metabolite in a control subject or population (e.g. in a sample that has been obtained from a control subject or population). Appropriate control subjects or samples for use in the methods of the invention would be readily identified by a person skilled in the art. Such subjects might also be referred to as “normal” subjects or as a reference population. Examples of appropriate populations of control subjects would include healthy subjects, for example, individuals who have no history of any form of cancer and no other concurrent disease, or subjects who are not suffering from, and preferably have no history of suffering from, any form of cancer. Preferably control subjects are not regular users of any medication. In a preferred embodiment control subjects are healthy subjects.

The control level may correspond to the level of the equivalent expression product or related metabolite in appropriate control subjects or samples, e.g. may correspond to a cut-off level or range found in a control or reference population. Alternatively, said control level may correspond to the level of the marker (expression product or related metabolite) in question in the same individual subject, or a sample from said subject, measured at an earlier time point (e.g. comparison with a “baseline” level in that subject). This type of control level (i.e. a control level from an individual subject) is particularly useful for embodiments of the invention where serial or periodic measurements of expression product (or related metabolite) levels in individuals, either healthy or ill, are taken looking for changes in the levels of the expression product(s) (or related metabolite(s)). In this regard, an appropriate control level will be the individual's own baseline, stable, nil, previous or dry value (as appropriate) as opposed to a control or cutoff level found in the general control population. Control levels may also be referred to as “normal” levels or “reference” levels. The control level may be a discrete figure or a range.

Although the control level for comparison could be derived by testing an appropriate set of control subjects, the methods of the invention would not necessarily involve carrying out active tests on control subjects as part of the methods of the present invention but would generally involve a comparison with a control level which had been determined previously from control subjects and was known to the person carrying out the methods of the invention.

A control level may be the level of an expression product (or related metabolite) in a healthy tissue sample (control tissue sample) of a control subject or population, where the healthy tissue sample is from the same tissue as the test sample (i.e. the potentially cancerous sample) being screened. Purely by way of example, if the test sample being screened is a breast tissue sample, the control level may be the level in a normal (e.g. healthy) breast tissue sample.

The methods of the present invention can be carried out on any appropriate sample. Typically the sample has been obtained from (removed from) a subject, preferably a human subject. In other aspects, the method further comprises a step of obtaining a sample from the subject. In some embodiments, the sample is a tissue sample from a subject (e.g. a tissue biopsy from a tissue suspected of being cancerous).

Any sample that is directly or indirectly affected by the suspected cancer (e.g. tumour) may be used. In some embodiments, the sample is blood or plasma. A plasma sample may comprise DNA and/or RNA from circulating tumour cells and or proteins and/or metabolites that have diffused from a tumour. In some embodiments, a sample may comprise circulating tumour cells. In some embodiments the sample is urine. Urine samples may comprise DNA and/or RNA and/or proteins and/or metabolites that have diffused from a tumour.

The term “sample” also encompasses any material derived by processing a biological sample. Derived materials include, but are not limited to, cells isolated from the sample, cell components, proteins/peptides and nucleic acid molecules (DNA or RNA) extracted from the sample. Processing of biological samples to obtain a test sample may involve one or more of: filtration, distillation, centrifugation, extraction, concentration, dilution, purification, inactivation of interfering components, addition of reagents, and the like. In some embodiments, methods of the invention may thus be carried out on samples which have been processed in some way (e.g. are man-made rather than native samples). Such samples may contain one or more buffers, diluents or the like.

In some embodiments, methods of the invention may include a step of processing a sample. In some embodiments, methods of the invention may thus be performed on such processed samples or materials derived from such processed samples. Processing steps include, but are not limited to, isolating cells from the sample, isolating cell components from the sample, extracting (e.g. isolating or purifying) proteins/peptides and/or nucleic acid molecules (DNA or RNA) and/or metabolites from the sample. A processing step may involve one or more of filtration, distillation, centrifugation, extraction, concentration, dilution, purification, inactivation of interfering components, addition of reagents, derivatization, amplification, adapter ligation, and the like.

The methods of screening, diagnosis, treatment etc., of the present invention are for cancer. In some embodiments the cancer is a tumour (e.g. a solid tumour). In some embodiments the cancer is breast cancer (e.g. epithelial breast cancer or breast carcinoma). In some embodiments the cancer is colon cancer (e.g. colon adenocarcinoma). In some embodiments the cancer is head and neck cancer (e.g. head and neck squamous cell carcinoma). In some embodiments the cancer is lung cancer (e.g. lung adenocarcinoma or lung squamous cell carcinoma). In some embodiments the cancer is uterine cancer (e.g. uterine corpus endometrial cancer). In some embodiments the cancer is oesophageal cancer. In some embodiments the cancer is bladder cancer (e.g. bladder adenocarcinoma). In some embodiments the cancer is glioblastoma multiforme. In some embodiments the cancer is kidney cancer (e.g. clear cell renal cell carcinoma). In some embodiments the cancer is glioma (e.g. low grade glioma). In some embodiments the cancer is ovarian cancer (e.g. ovarian carcinoma). In some embodiments the cancer is rectal cancer (e.g. rectum adenocarcinoma). In some embodiments the cancer is pancreatic cancer (e.g. pancreatic adenocarcinoma.

In some embodiments the cancer is not colorectal cancer.

The methods of the invention as described herein can be carried out on any type of subject which is capable of suffering from cancer. The methods are generally carried out on mammals, for example humans, primates (e.g. monkeys), laboratory mammals (e.g. mice, rats, rabbits, guinea pigs), livestock mammals (e.g. horses, cattle, sheep, pigs) or domestic pets (e.g. cats, dogs). Preferably, the subject is a human.

In one embodiment, the subject (e.g. a human) is a subject at risk of developing cancer or at risk of the occurrence of cancer (e.g. a healthy subject or a subject not displaying any symptoms of cancer or any other appropriate “at risk” subject). In another embodiment the subject is a subject having, or suspected of having (or developing), or potentially having (or developing), cancer.

In some aspects, a method of the invention may further comprise an initial step of selecting a subject (e.g. a human subject) at risk of developing cancer or having, or suspected of having (or developing), or potentially having (or developing), cancer. The subsequent method steps can be performed on a sample from such a selected subject.

A yet further aspect provides a kit for the screening (e.g. diagnosis or prognosis) of cancer which comprises an agent suitable for determining the level of an expression product (or related metabolite) of one or more of the AraX genes described above, or fragments thereof, in a sample. Preferred agents are antibodies. Other suitable agents, if the expression product is a nucleic acid molecule, include oligonucleotide primers (e.g. primer pairs) and/or probes that recognise at least a portion of a target nucleic acid sequence. In preferred aspects said kits are for use in the methods of the invention as described herein. Preferably, said kits comprise instructions for use of the kit components, for example in diagnosis. In some embodiments, the kit is a multimarker kit. Thus, in some embodiments the kit comprises more than one agent (e.g. two, three or four distinct agents), each agent being suitable for determining the level of one of the expression products (or related metabolites) described above, or fragments thereof, in a sample. Using such kits (multimarker kits) the level of multiple (e.g. two, three or four) expression products (or related metabolites) may be determined. Exemplary groups (combinations) of AraX genes are discussed elsewhere herein in relation to other aspects of the invention. In some embodiments the level of expression products (or related metabolites) of such groups of AraX genes may be determined using such multimarker kits. In a preferred embodiment of such multimarker kits, the agent suitable for determining the level of an expression product (or related metabolite) is an antibody.

In another aspect, the present invention provides a solid support (e.g. a chip) comprising a group of one or more probes (e.g. nucleic acid probes) capable of detecting the presence or level of an expression product (e.g. nucleic acid expression product) of one or more of the genes or groups of genes described herein. In some embodiments, said group of one or more probes comprises or consists of at least 2, at least 5, at least 10, at least 20, at least 30, at least 40, at least 50, at least 60, at least 70, at least 80, or up to 84 probes (e.g. 2-84, 5-84 or 10-84 or 20-84 or 30-84 or 40-84 or 50-84). In some embodiments, said group of one or more probes comprises or consists of up to 5, up to 10, up to 20, up to 30, up to 40, up to 50, up to 60, up to 70, up to 80 or up to 84 probes.

In one aspect, the present invention provides a method of detecting (or determining) the level of an expression product (or related metabolite) of one or more genes of the AraX network as set forth elsewhere herein, wherein said sample has been obtained from said subject.

In one aspect, the present invention provides a method of detecting the level of an expression product (or related metabolite) of one or more genes of the AraX network as set forth elsewhere herein, said method comprising:
(a) obtaining a sample from a human patient; and
(b) detecting the level of an expression product (or related metabolite) of one or more of said genes in said sample.

The features and discussion herein in relation to the method of screening for cancer (e.g. method of diagnosing, method for prognosis etc.), for example in relation to preferred genes or combinations thereof for measurement, can be applied, mutatis mutandis, to methods of detecting of the present invention.

Table 1 shows the official gene symbols and official (approved) gene names of the AraX genes.

| | Approved | | UniProt |
|---|---|---|---|
| HGNC ID | symbol | Approved name | accession |
| HGNC:51 | ABCC1 | ATP binding cassette subfamily C member 1 | P33527 |
| HGNC:53 | ABCC2 | ATP binding cassette subfamily C member 2 | Q92887 |
| HGNC:54 | ABCC3 | ATP binding cassette subfamily C member 3 | O15438 |
| HGNC:16526 | ACSL5 | acyl-CoA synthetase long-chain family member 5 | Q9U LC5 |
| HGNC:251 | ADH1C | alcohol dehydrogenase 1C (class 1), gamma polypeptide | P00326 |
| HGNC:255 | ADH6 | alcohol dehydrogenase 6 (class V) | P28332 |
| HGNC:256 | ADH7 | alcohol dehydrogenase 7 (class IV), mu or sigma polypeptide | P40394 |
| HGNC:16354 | ADHFE1 | alcohol dehydrogenase, iron containing 1 | Q8IWW8 |
| HGNC:382 | AKR1B10 | aldo-keto reductase family 1, member B10 (aldose reductase) | O60218 |
| HGNC:37281 | AKR1B15 | aldo-keto reductase family 1, member B15 | C9JRZ8 |
| HGNC:384 | AKR1C1 | aldo-keto reductase family 1, member C1 | Q04828 |
| HGNC:385 | AKR1C2 | aldo-keto reductase family 1, member C2 | P52895 |
| HGNC:386 | AKR1C3 | aldo-keto reductase family 1, member C3 | P42330 |
| HGNC:405 | ALDH3A1 | aldehyde dehydrogenase 3 family member A1 | P30838 |
| HGNC:403 | ALDH3A2 | aldehyde dehydrogenase 3 family member A2 | P51648 |
| HGNC:410 | ALDH3B1 | aldehyde dehydrogenase 3 family member B1 | P43353 |
| HGNC:435 | ALOX5 | arachidonate 5-lipoxygenase | P09917 |
| HGNC:433 | ALOX15 | arachidonate 15-lipoxygenase | P16050 |
| HGNC:80 | AOC1 | amine oxidase, copper containing 1 | P19801 |
| HGNC:1548 | CBR1 | carbonyl reductase 1 | P16152 |
| HGNC:1549 | CBR3 | carbonyl reductase 3 | O75828 |
| HGNC:1795 | CDO1 | cysteine dioxygenase type 1 | Q16878 |
| HGNC:1863 | CES1 | carboxylesterase 1 | P23141 |
| HGNC:2295 | CP | ceruloplasmin (ferroxidase) | P00450 |
| HGNC:2631 | CYP2E1 | cytochrome P450 family 2 subfamily E member 1 | P35222 |
| HGNC:15654 | CYP2S1 | cytochrome P450 family 2 subfamily S member 1 | P05181 |
| HGNC:20243 | CYP2W1 | cytochrome P450 family 2 subfamily W member 1 | Q96SQ9 |
| HGNC:2638 | CYP3A5 | cytochrome P450 family 3 subfamily A member 5 | Q8TAV3 |
| HGNC:2644 | CYP4B1 | cytochrome P450 family 4 subfamily B member 1 | P20815 |
| HGNC:2646 | CYP4F3 | cytochrome P450 family 4 subfamily F member 3 | P13584 |
| HGNC:13265 | CYP4F11 | cytochrome P450 family 4 subfamily F member 11 | Q08477 |
| HGNC:20244 | CYP4X1 | cytochrome P450 family 4 subfamily X member 1 | Q9HBI6 |
| HGNC:2602 | CYP24A1 | cytochrome P450 family 24 subfamily A member 1 | Q8N118 |
| HGNC:2605 | CYP27A1 | cytochrome P450 family 27 subfamily A member 1 | Q07973 |
| HGNC:2606 | CYP2761 | cytochrome P450 family 27 subfamily B member 1 | Q02318 |
| HGNC:17449 | CYP39A1 | cytochrome P450 family 39 subfamily A member 1 | O15528 |
| HGNC:14416 | ELOVL2 | ELOVL fatty acid elongase 2 | Q9NYL5 |
| HGNC:3401 | EPHX1 | epoxide hydrolase 1 | Q9NXB9 |
| HGNC:26440 | FAAH2 | fatty acid amide hydrolase 2 | P07099 |
| HGNC:3769 | FMO1 | flavin containing monooxygenase 1 | Q6GMR7 |
| HGNC:3771 | FMO3 | flavin containing monooxygenase 3 | Q01740 |
| HGNC:3772 | FMO4 | flavin containing monooxygenase 4 | P31513 |
| HGNC:3773 | FMO5 | flavin containing monooxygenase 5 | P31512 |
| HGNC:4311 | GCLC | glutamate-cysteine ligase, catalytic subunit | P49326 |
| HGNC:4312 | GCLM | glutamate-cysteine ligase modifier subunit | P48506 |
| HGNC:26891 | GGT6 | gamma-glutamyltransferase 6 | P48507 |
| HGNC:4554 | GPX2 | glutathione peroxidase 2 | Q6P531 |
| HGNC:4555 | GPX3 | glutathione peroxidase 3 | P18283 |
| HGNC:4623 | GSR | glutathione reductase | P22352 |
| HGNC:4627 | GSTA2 | glutathione S-transferase alpha 2 | P00390 |
| HGNC:4632 | GSTM1 | glutathione S-transferase mu 1 | P09210 |
| HGNC:4634 | GSTM2 | glutathione S-transferase mu 2 (muscle) | P09488 |
| HGNC:4635 | GSTM3 | glutathione S-transferase mu 3 (brain) | P28161 |
| HGNC:4636 | GSTM4 | glutathione S-transferase mu 4 | P21266 |
| HGNC:13312 | GSTO1 | glutathione S-transferase omega 1 | Q03013 |
| HGNC:23064 | GSTO2 | glutathione S-transferase omega 2 | P78417 |
| HGNC:4892 | HGD | homogentisate 1,2-dioxygenase | Q9H4Y5 |
| HGNC:5154 | HPGD | hydroxyprostaglandin dehydrogenase 15-(NAD) | Q93099 |
| HGNC:17890 | HPGDS | hematopoietic prostaglandin D synthase | P15428 |
| HGNC:6719 | LTC4S | leukotriene C4 synthase | O60760 |
| HGNC:6834 | MAOB | monoamine oxidase B | O75874 |
| HGNC:25193 | MBOAT2 | membrane bound O-acyltransferase domain containing 2 | Q14145 |
| HGNC:7061 | MGST1 | microsomal glutathione S-transferase 1 | Q16873 |
| HGNC:21063 | MOXD1 | monooxygenase, DBH-like 1 | P27338 |
| HGNC:2874 | NQO1 | NAD(P)H dehydrogenase, quinone 1 | Q6ZWT7 |
| HGNC:7856 | NQO2 | NAD(P)H dehydrogenase, quinone 2 | P10620 |
| HGNC:8149 | OPLAH | 5-oxoprolinase (ATP-hydrolysing) | Q6UVY6 |
| HGNC:9031 | PLA2G2A | phospholipase A2 group IIA | Q16236 |
| HGNC:9035 | PLA2G4A | phospholipase A2 group IVA | P15559 |
| HGNC:24791 | PLA2G4E | phospholipase A2 group IVE | P16083 |
| HGNC:9039 | PLA2G6 | phospholipase A2 group VI | Q96L73 |
| HGNC:9029 | PLA2G10 | phospholipase A2 group X | O14841 |
| HGNC:18554 | PLA2G12A | phospholipase A2 group XIIA | P14555 |
| HGNC:9599 | PTGES | prostaglandin E synthase | P47712 |
| HGNC:18429 | PTGR1 | prostaglandin reductase 1 | Q3MJ16 |
| HGNC:9604 | PTGS1 | prostaglandin-endoperoxide synthase 1 (prostaglandin G/H | O60733 |
| | | synthase and cyclooxygenase) | |
| HGNC:9605 | PTGS2 | prostaglandin-endoperoxide synthase 2 | O15496 |
| HGNC:10961 | SLCO1B3 | solute carrier organic anion transporter family member 1B3 | Q9BZM1 |
| HGNC:10955 | SLCO2A1 | solute carrier organic anion transporter family member 2A1 | P60484 |
| HGNC:11453 | SULT1A1 | sulfotransferase family 1A member 1 | O14684 |
| HGNC:11454 | SULT1A2 | sulfotransferase family 1A member 2 | Q14914 |
| HGNC:30004 | SULT1A4 | sulfotransferase family 1A member 4 | P23219 |
| HGNC:12530 | UGT1A1 | UDP glucuronosyltransferase 1 family, polypeptide A1 | P35354 |
| HGNC:12538 | UGT1A6 | UDP glucuronosyltransferase 1 family, polypeptide A6 | P06400 |

Table 2 shows the official gene symbols and official (approved) gene names of certain genes that are commonly mutated in cancer.

| | Approved | | UniProt |
|---|---|---|---|
| HGNC ID | symbol | Approved name | accession |
| HGNC:2514 | CTNNB1 | catenin beta 1 | Q9NPD5 |
| HGNC:5382 | IDH1 | isocitrate dehydrogenase 1 (NADP+) | Q92959 |
| HGNC:23177 | KEAP1 | kelch like ECH associated protein 1 | Q15831 |
| HGNC:7782 | NFE2L2 | nuclear factor, erythroid 2 like 2 | P50225 |
| HGNC:14234 | NSD1 | nuclear receptor binding SET domain | P50226 |
| | | protein 1 | |
| HGNC:9588 | PTEN | phosphatase and tensin homolog | P0DMN0 |
| HGNC:9884 | RB1 | retinoblastoma 1 | P04637 |
| HGNC:11389 | STK11 | serine/threonine kinase 11 | P22309 |
| HGNC:11998 | TP53 | tumor protein p53 | P19224 |

The invention will be further described with reference to the following non-limiting Example with reference to the following drawings.

The patent or application file contains at least one drawing executed in color. Copies of this patent or patent application publication with color drawing(s) will be provided by the Office upon request and payment of the necessary fee.

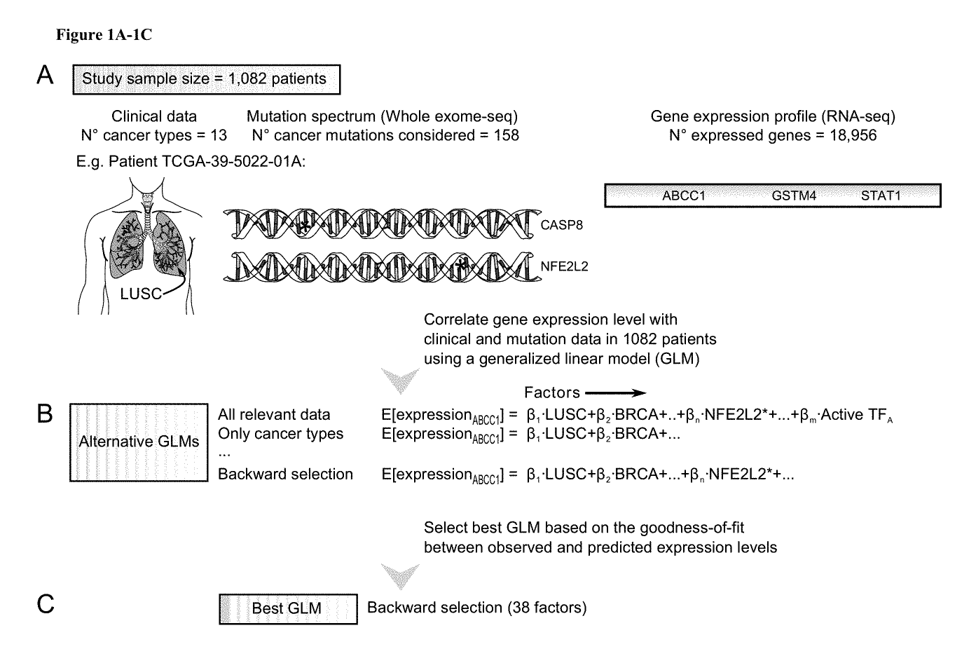

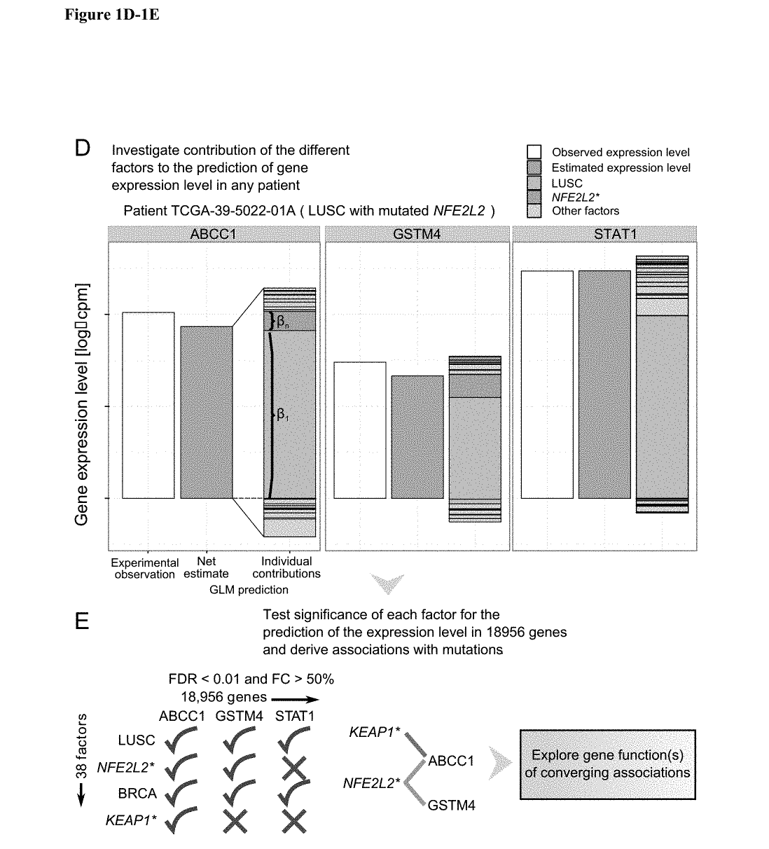

FIGS. 1A-1E: Workflow used to derive statistical associations between gene expression changes and cancer mutations. (FIG. 1A) Input data for the study were collected from 1,082 patients for which clinical, mutation, and gene expression level data were simultaneously generated. LUSC: Lung squamous cell carcinoma. (FIG. 1B) The observed level of gene expression was correlated to clinical and mutation data by deriving alternative generalized linear models (GLMs). Each GLM factorizes the contribution of predefined factors to the expression level of a given gene (e.g. ABCC1) as a linear regression, where coefficients are estimated by fitting the observed gene expression level in the 1,082 samples. Each GLM predicts an expected value for the expression level of a gene in a sample given the factor values for that sample (e.g. if the sample is LUSC, the GLM adds a contribution equal to its estimated coefficient, β₁). (FIG. 1C) Model selection is performed to decide which GLM returns the best predictions while using a minimal number of factors. (FIG. 1D) The predicted expression is net sum of positive and negative factors as determined by the model. As example, expression of ABCC1 is positively affected by a cancer type factor (LUSC) and a mutation in NFE2L2. (FIG. 1E) The significance of each factor can be tested using a threshold for the moderated t-statistics and for the minimum expression fold-change. The factors representing mutations can hereby be associated with gene expression changes. For example, a mutation in NFE2L2 showed a significant statistical association with expression changes in ABCC1. Associations identified in this manner were used to derive networks of deregulated biological processes that are independently associated with cancer mutations.

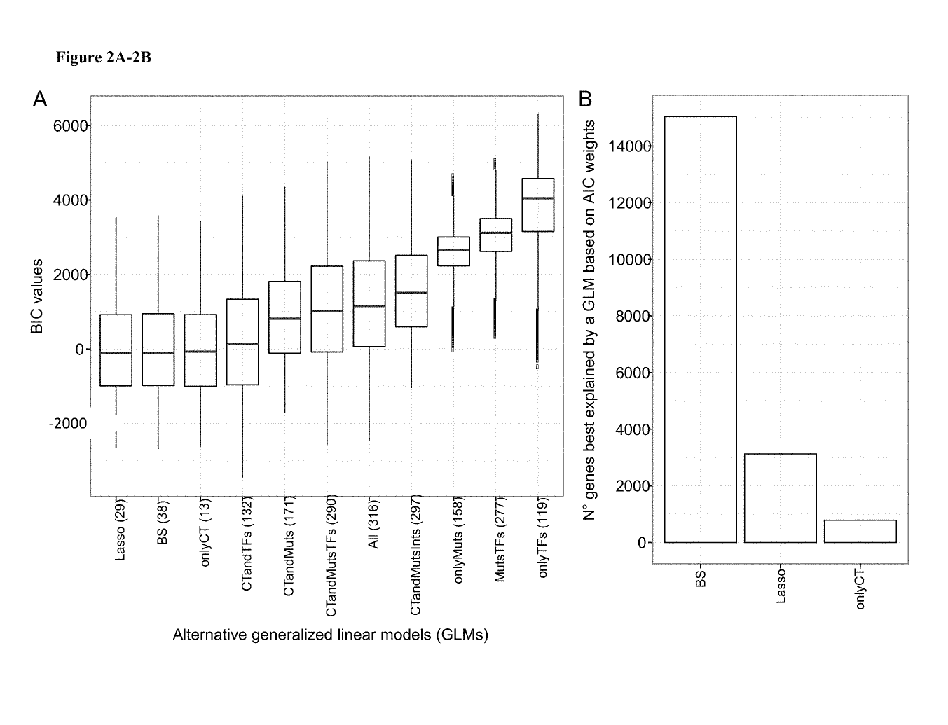

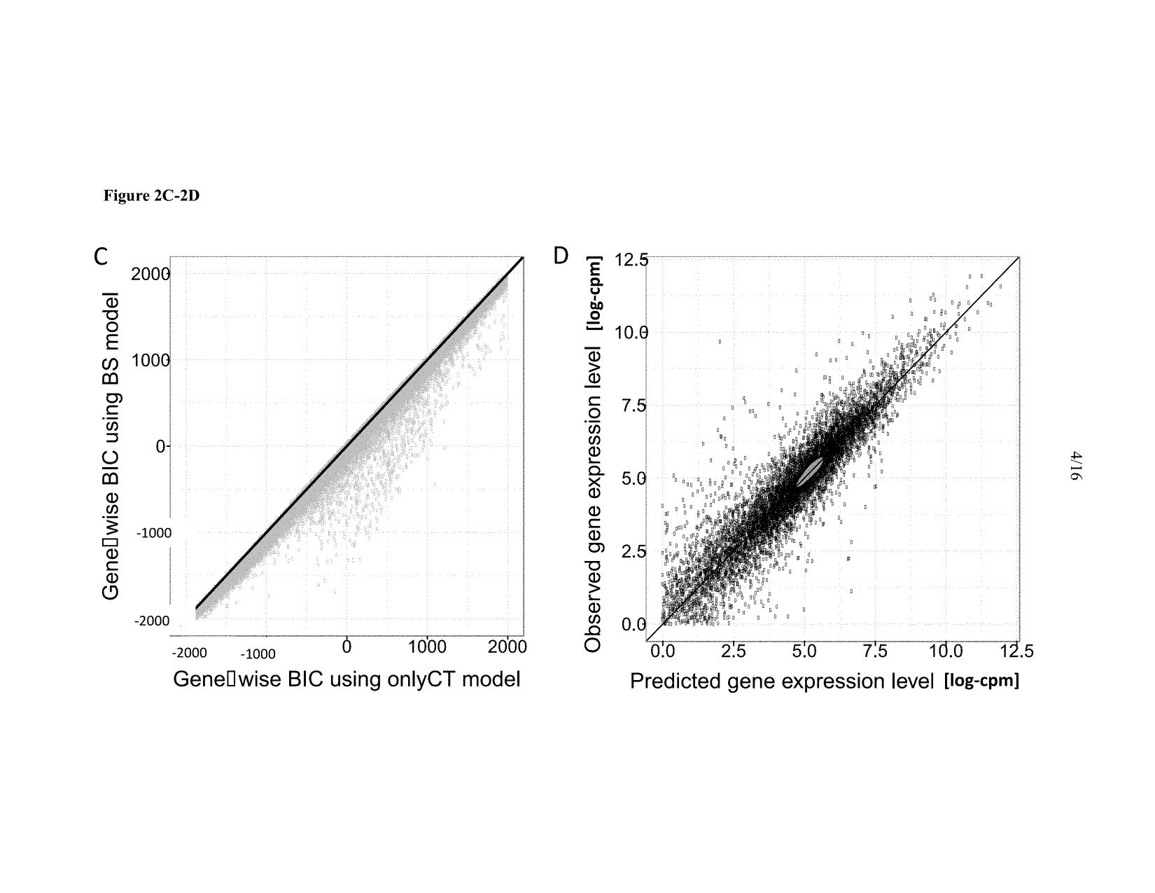

FIGS. 2A-2D: Model selection according to the minimum Bayesian or Akaike information criterion (BIC or AIC) reveals that the backward selection (BS) model is better at fitting gene expression across samples than the alternative GLMs. (FIG. 2A) Boxplot of BIC values (one for each gene) using alternative GLMs. Key: Lasso—Lasso non null factors in >0.5% of all genes (29 factors); BS—Backward selection model (38 factors); CT—Cancer type factors (13 factors); TFs—Transcription factor expression level factors (119 factors); Muts—Presence of a mutation in cancer genes (158 factors); Ints—Interaction term between presence of a mutated gene and cancer type (126 factors); All—All factors (316 factors). (FIG. 1B) Number of genes whose expression is best explained by one of the alternative GLMs based on BIC weights. (FIG. 1C) Comparison of the BIC value for the regression of expression of each individual gene using either the onlyCT or the BS model. Bluer contours define areas with increasing density of points. (FIG. 1D) Correlation between observed and predicted gene expression levels using the BS model. Bluer contours define areas with increasing density of points.

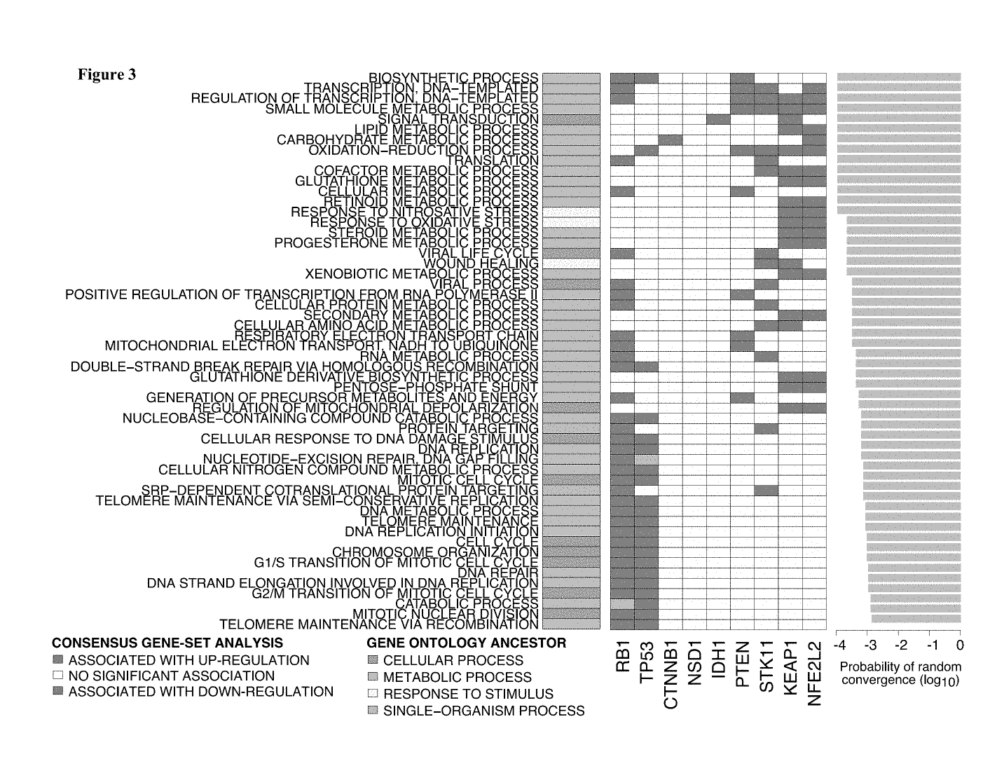

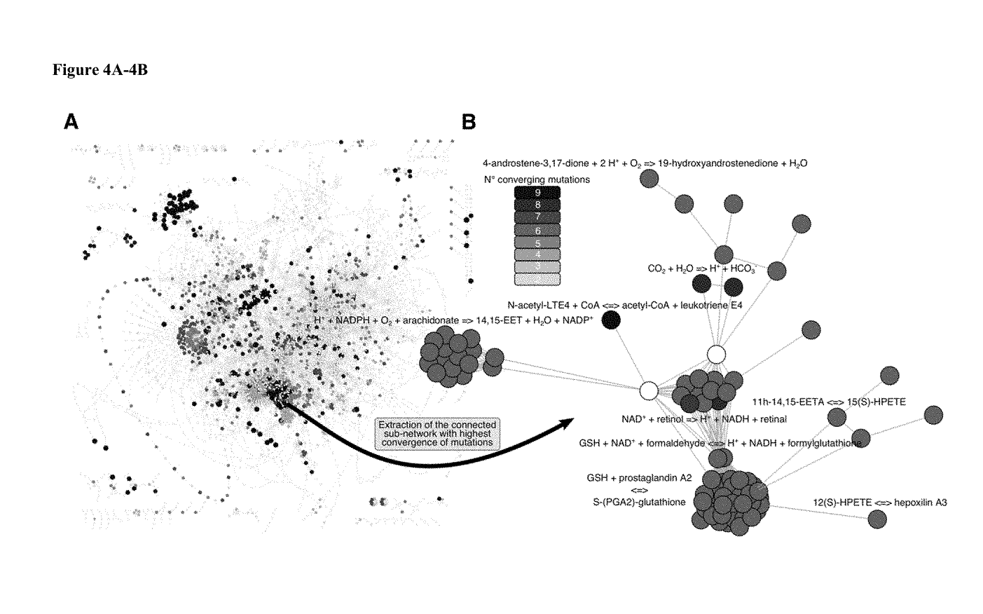

FIG. 3: Mutated genes converge on the regulation of GO biological processes that relate primarily to metabolism. Each row indicates a GO term that is enriched in the up- (red) or down- (blue) regulated genes associated with each mutated gene (column) in the consensus gene-set analysis. GO terms are classified according to the ancestor GO category and sorted by the significance of the convergence (barplot on the right).

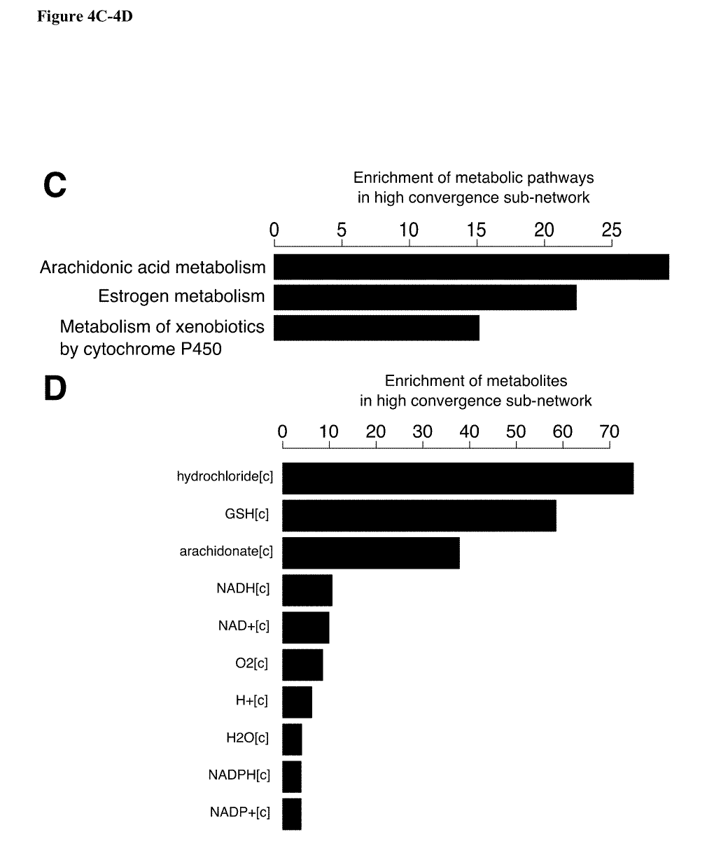

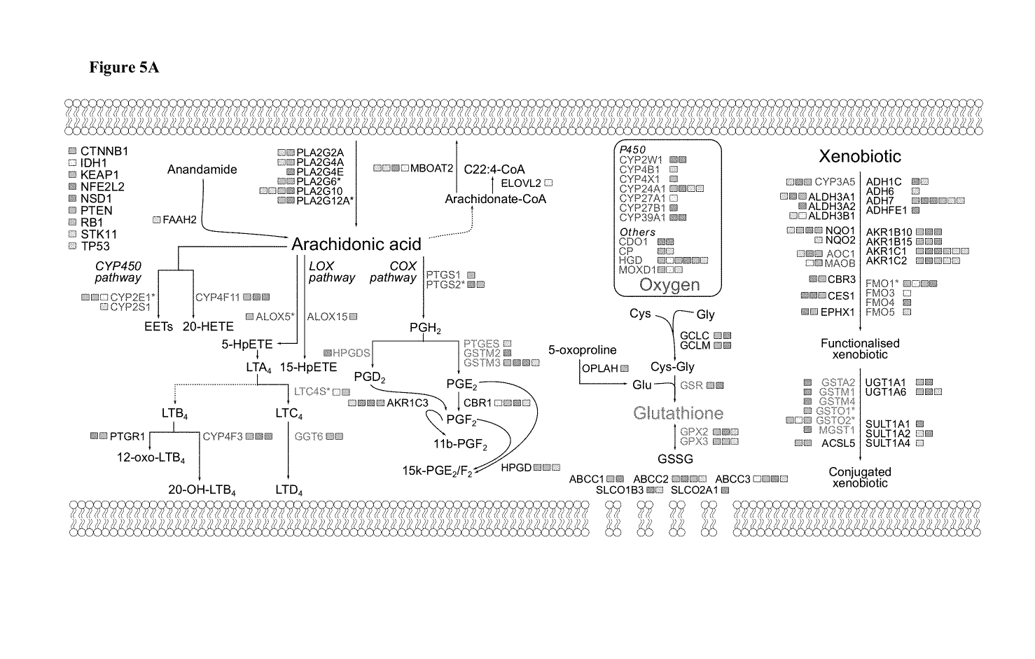

FIGS. 4A-4D: The network of associations between cancer mutations and metabolic genes reveals a region of high convergence in which genes encode for a metabolic sub-network revolving around arachidonic acid and xenobiotics. (FIG. 4A) The human metabolic reaction network where each node is a reaction and the blue gradient indicates the number of mutated genes converging to it via association with any reaction-encoding gene. (FIG. 4B) Extraction of the sub-network where the number of converging mutation-driven transcriptional changes is maximized. (FIG. 4C-D) Characterization of the sub-network in terms of over-represented pathways (FIG. 4C) and metabolites (FIG. 4D) compared to the background human metabolic network.

FIGS. 5A-5C: A literature curated sub-network of reactions that revolves around arachidonic acid and xenobiotic metabolism (AraX) shows convergence by multiple mutated genes in cancer (FIG. 5A). The boxes next to each gene indicate which mutated genes are associated with it. (FIG. 5B-C) Overrepresentation of AraX compared to KEGG (FIG. 5B) or Reactome (FIG. 5C) metabolic pathways by genes associated with a mutated gene. Each bar indicates the odds ratio for the corresponding mutation. The top five ranked pathways are sorted according to mean overrepresentation (grey bar), where the error bars span the 95% bootstrap confidence interval.

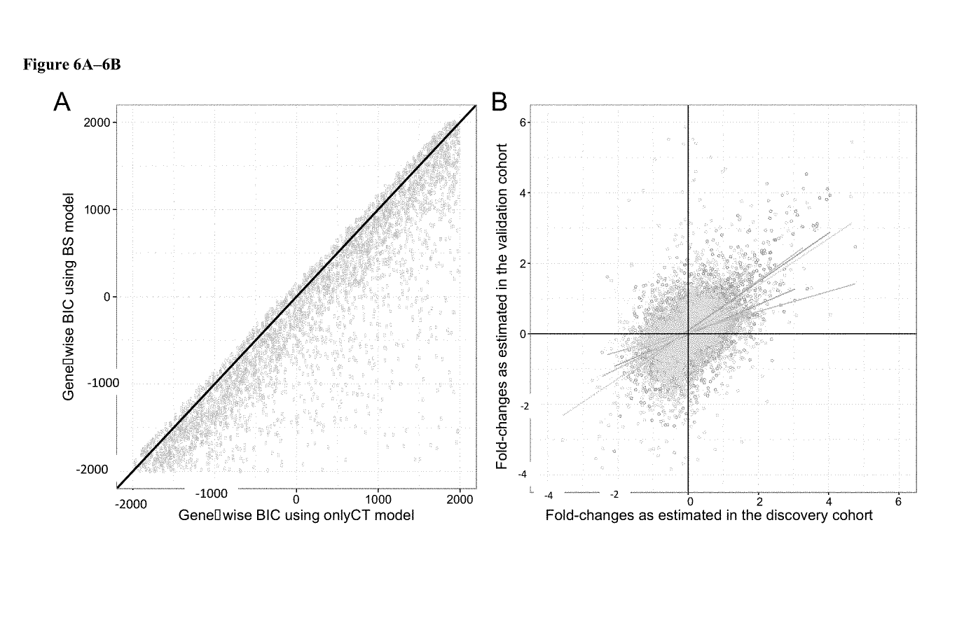

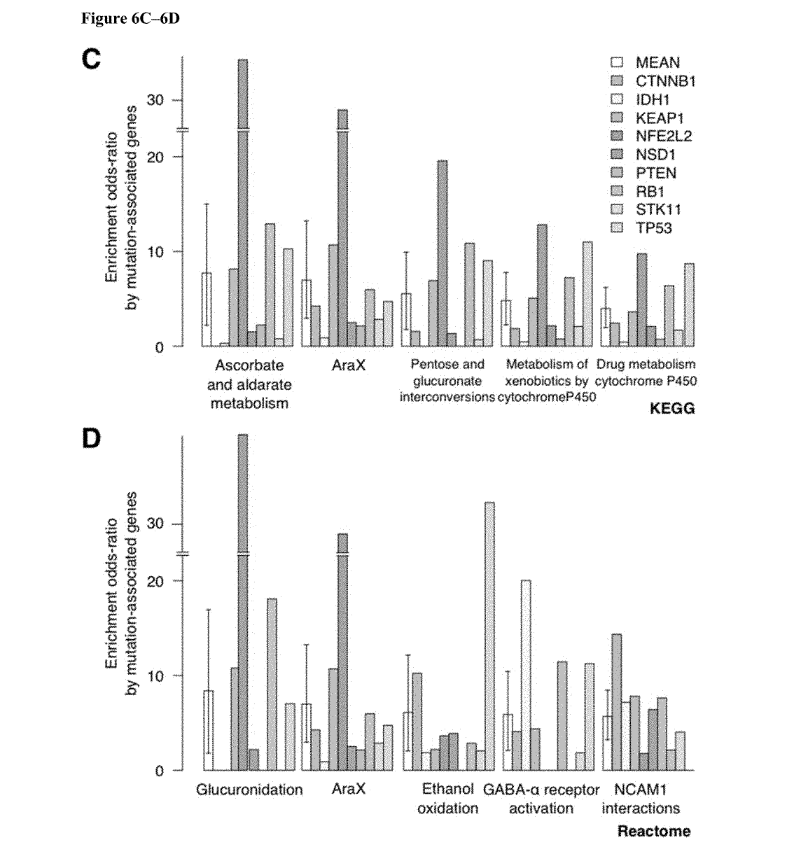

FIGS. 6A-6D: Validation of the associations between mutated genes and gene expressions and their convergence on AraX deregulation in an independent cohort of 4,462 samples. (FIG. 6A) Comparison of the BIC value for the regression of expression of each individual gene using either the onlyCT or the BS model. Bluer contours define areas with increasing density of points. (FIG. 6B) Correlation of expression fold-changes for mutation-associated genes as estimated using either the discovery or the validation cohort (each color defines genes associated with a given mutated gene and the linear interpolation between fold-changes estimated in the two independent cohorts). (FIG. 6C-D) Overrepresentation of AraX compared to KEGG (FIG. 6C) or Reactome (FIG. 6D) metabolic pathways by genes associated with a mutated gene in the validation cohort. Each bar indicates the odds ratio for the corresponding mutation. The top five ranked pathways are sorted according to mean overrepresentation (grey bar), where the error bars span the 95% bootstrap confidence interval.

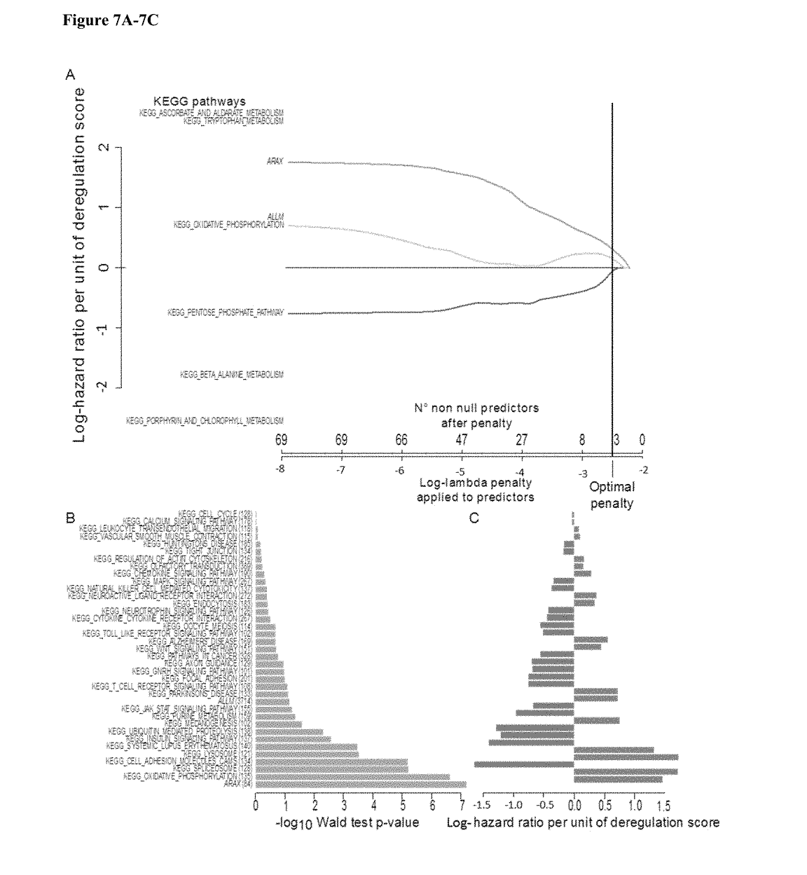

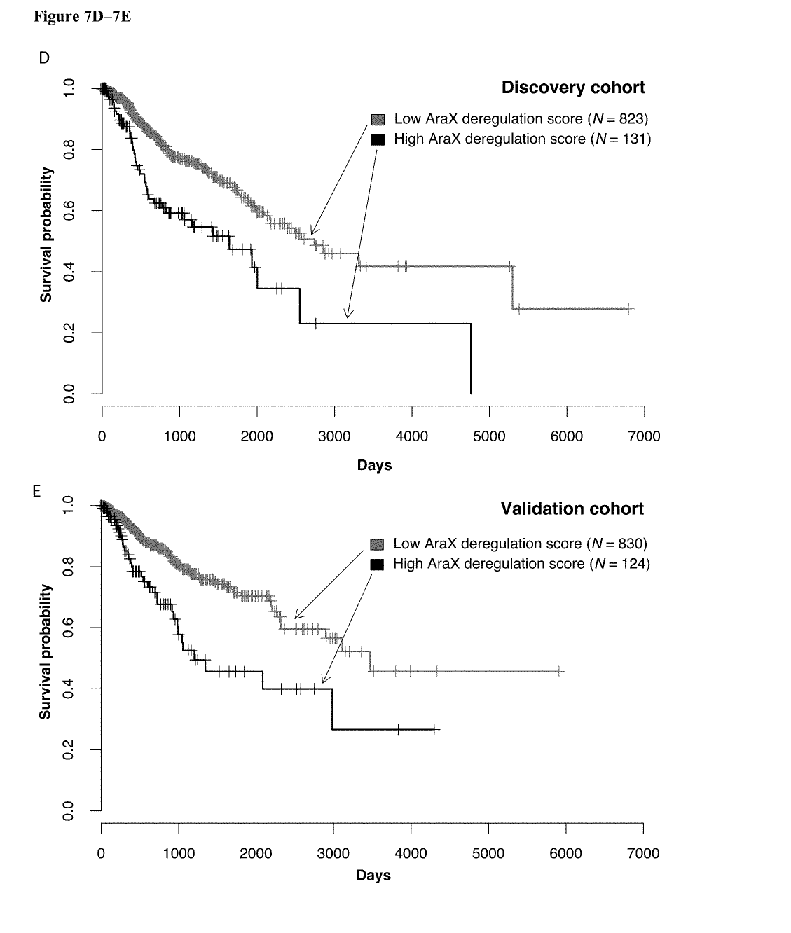

FIGS. 7A-7E: Survival analysis of patients stratified upon metabolic pathway deregulation reveals AraX as the strongest predictor of survival. (FIG. 7A) Log-hazard ratio per unit of deregulation score for AraX, 186 KEGG metabolic pathways, and a geneset including 3714 metabolic genes) at different Lasso penalties (log-X) in the multivariate prediction of overall survival for 718 tumors. Each path represents a different pathway. In colors only the paths relative to pathways that are predictive of survival at the optimal lasso penalty, log-λ=−2.5 (vertical line), the remaining paths are shown as grey. The graph shows that AraX is the strongest predictor of survival at the optimal lasso penalty, followed by oxidative phosphorylation and the pentose phosphate pathway and that its predictive strength is robust to different choices of lasso penalties. (FIG. 7B) Wald test statistic in the univariate Cox regression of survival using deregulation of the pathways in (FIG. 7A) that contain at least 100 genes. (FIG. 7C) Log-hazard ratio per unit of deregulation score for the pathways in (FIG. 7B). (FIG. 7D-E) Kaplan-Meier survival plots for 1,908 tumor samples equally split in a discovery (FIG. 7D) and validation (FIG. 7E) cohort and stratified upon “low” (grey) versus “high” (black) AraX deregulation score according to a threshold derived in the discovery cohort.

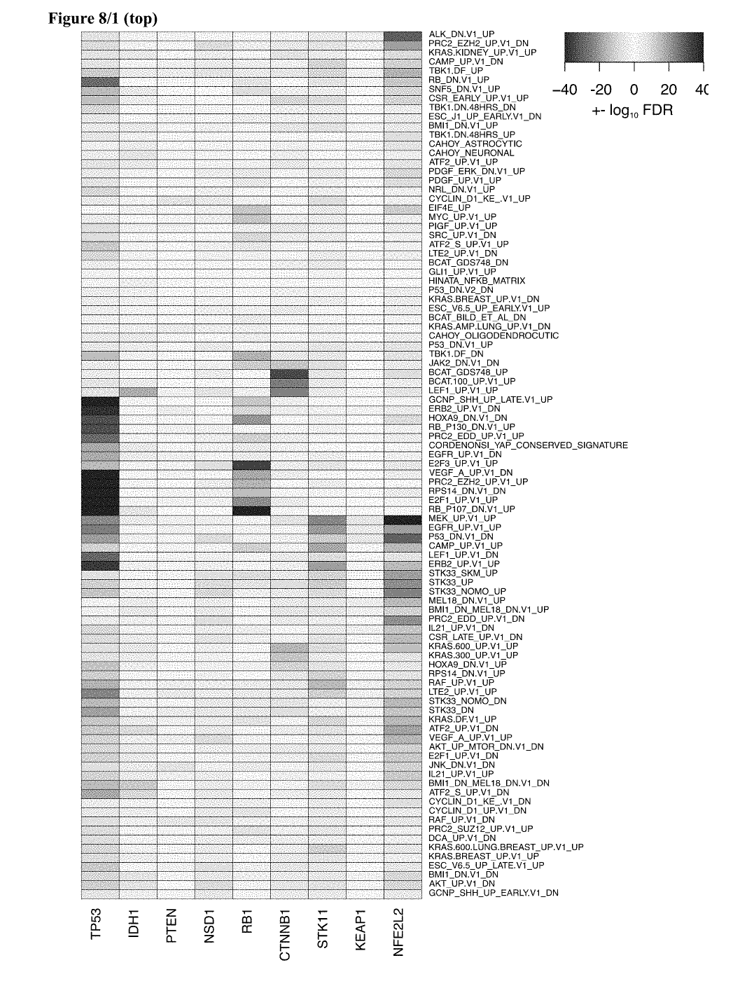

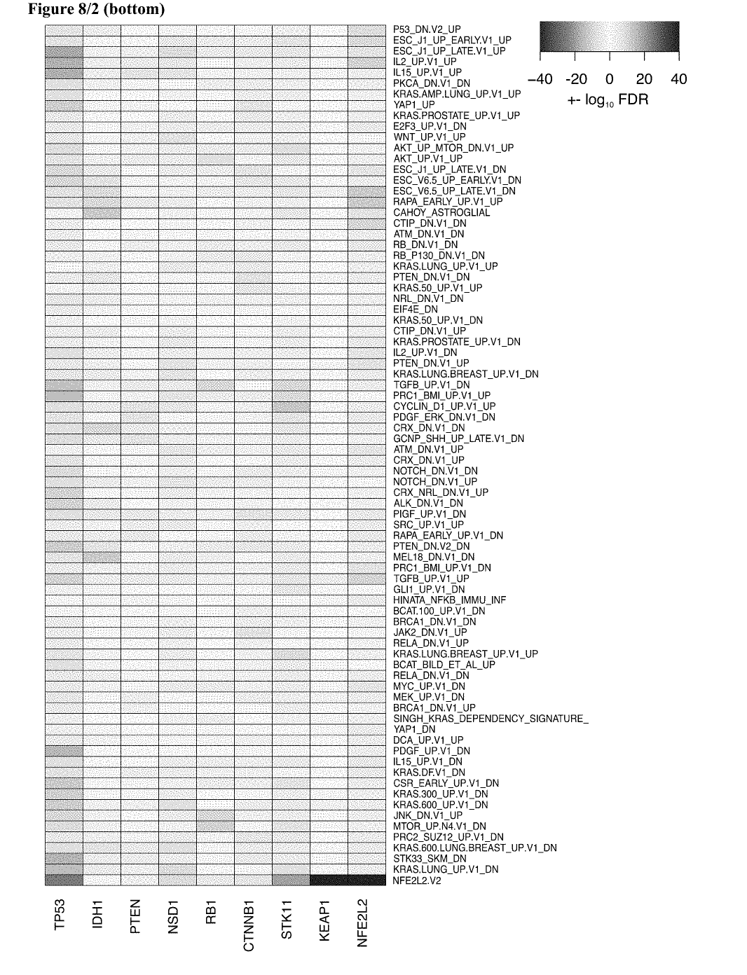

FIG. 8: Heatmap of a gene-set analysis for a list of gene-sets representing each a genetic perturbation in a key cancer-associated gene using the genes found here to be associated with a mutated gene reveals high consistency (e.g. genes here found up-regulated when APC is mutated significantly enrich the BCAT_BILD_ET_AL_UP gene-set, where β-catenin, a direct target of APC, is over-expressed in primary epithelial breast cancer cell; or genes here found down-regulated when TP53 is mutated significantly enrich the P53_DN.V1_DN gene-set, which features down-regulated genes in a NCI-60 panel of cell lines with mutated TP53).

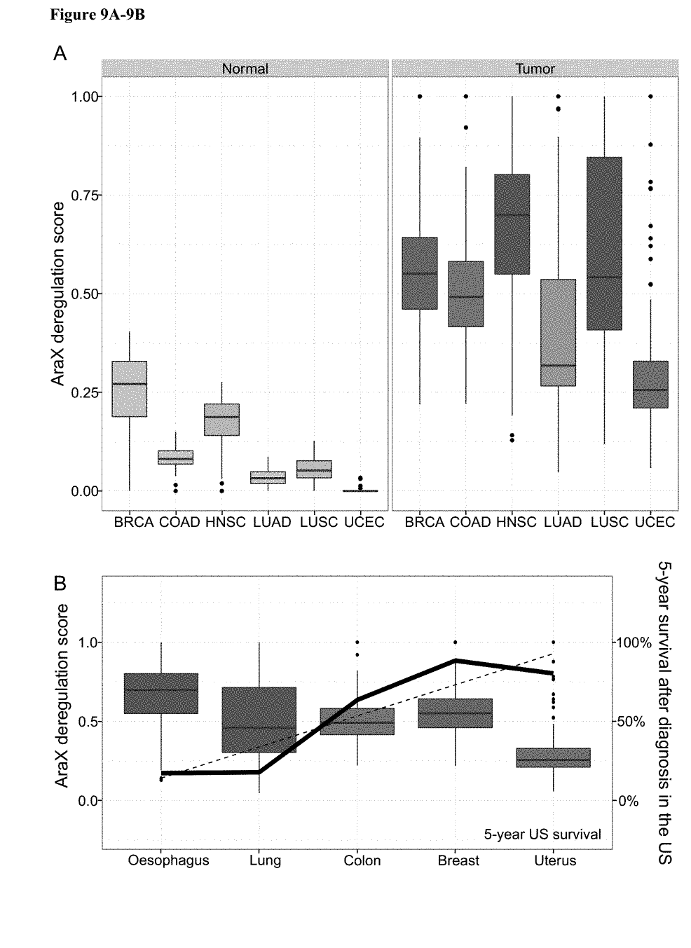

FIGS. 9A-9B: (FIG. 9A) Boxplots of AraX deregulation score in normal vs. tumor samples, grouped by cancer type. Key: BRCA—Breast carcinoma, COAD—Colon adenocarcinoma, HNSC—Head and neck squamous cell carcinoma, LUAD—Lung adenocarcinoma, LUSC—Lung squamous cell carcinoma, UCEC—Uterine corpus endometrial carcinoma. (FIG. 9B) Association between 5-year cancer survival estimates in the US (black solid line) and AraX deregulation scores in cancer from the same tissues (boxplots).

### EXAMPLES

### Example 1

Mutations stand at the basis of the clonal evolution of most cancers. Nevertheless, a systematic analysis on whether mutations are selected in cancer because they lead to deregulation of specific biological processes independent of the cancer type is still lacking. In this invention, we correlated the genome and transcriptome of 1,082 primary tumor samples. We found that 9 commonly mutated genes were associated with substantial changes in gene expression, which primarily converged on metabolism. Further network analyses circumscribed the convergence to a network of reactions, termed AraX, that involve the glutathione- and oxygen-mediated metabolism of arachidonic acid and xenobiotics. In an independent cohort of 4,462 samples, all 9 mutated genes consistently correlated with deregulation of AraX. Moreover, among all metabolic pathways, AraX deregulation represented the strongest predictor for patient survival. These findings suggest that oncogenic mutations drive a selection process that converges on deregulation of the AraX network to gain growth advantage during cancer evolution.

### EXPERIMENTAL PROCEDURES

### Data Retrieval

RNAseq gene expression profiles and clinical data for 1,082 primary tumor samples encompassing 13 cancer types (BLCA—Bladder adenocarcinoma, BRCA—Breast carcinoma, COAD—Colon adenocarcinoma, GBM—Glioblastoma multiforme, HNSC—Head and neck squamous cell carcinoma, KIRC—Clear cell renal cell carcinoma, LGG—Low grade glioma, LUAD—Lung adenocarcinoma, LUSC—Lung squamous cell carcinoma, OV—Ovarian carcinoma, READ—Rectum adenocarcinoma, PAAD—Pancreatic adenocarcinoma, UCEC—Uterine corpus endometrial carcinoma) were downloaded from the Cancer Genome Atlas (TCGA) in November 2013. A second group of 4,462 primary tumor samples encompassing the same 13 cancer types were also downloaded from TCGA in August 2015. Mutation profiles for all samples in this study were obtained from the cBioPortal (Gao et al., 2013).

### Differential Gene Expression Analysis

RNAseq-generated read count tables were used to estimate gene expression in each sample in the pan-cancer cohort. To this end, we adopted voom, an approach that extends the generalized linear model (GLM) for microarray gene expression signals to analyze count-based expression data (Law et al., 2013). The gene-wise count variance is calculated from the linear regression of gene-wise observed log-counts across all samples in the cohort according to a number of factors (to be decided), and it is defined as the gene-wise residual standard deviation of the regression. If a lowess curve is fitted to square-root residual standard deviation as a function of mean log-counts, it is possible to predict the square-root standard deviation of each observation (i.e. log-counts for a given gene in a given sample) from this mean-variance trend. Differential gene expression analysis for each factor is then performed using the standard linear modeling procedure proposed by limma (Smyth, 2004), with the addition that the log-counts-per-million of each observation are corrected using the predicted variance as an inverse weight. Even if voom assumes that each observation is normally distributed, this method proved to outperform count-based approaches in differential expression analysis comparison studies. The significance of each factor in the regression of the expression of each gene is then tested using moderated t-statistics. So generated p-values were corrected for multiple testing by controlling the false discovery rate (FDR) across genes using the Benjamini and Hochberg correction and by adopting the nestedF correction across contrasts. A factor is deemed significant in the regression of the expression of a gene if it is associated to at least 50% fold change (|log₂FC|>1.5) with a FDR<0.01.

### Generalized Linear Model Selection

In order to perform the differential gene expression analysis above, it is required to define the factors for the regression. These factors are devoted to explain the biological variability of gene-wise counts across the samples in the pan-cancer cohort. They should capture the main contributions and some smaller contributions interesting to our investigation. Hence we tentatively selected the following factors for an initial design (All):

The cancer types, i.e. the belonging to a histopathologically defined cancer type among the 13 types in the cohort;

The mutation status of 158 cancer-associated genes. An initial list of 260 genes was generated by merging the Cancer5000 and Cancer5000-S lists in (Lawrence et al., 2014). We excluded HIST1H3B, HIST1H4E, and MLL4, which could not be uniquely mapped using the Ensembl v.73 annotation. Furthermore, 102 genes that were not mutated at moderate frequency in the cohort (>2%) were also excluded. For the purpose of this study, any type of mutation in these genes was sufficient to qualify the gene as mutated in the sample.

The activation status of 119 well-characterized transcription factors, defined by the belonging to a certain quintile of expression in the pan-cancer cohort.

The interaction terms between a cancer type and a cancer-associated gene mutated at high frequency. These are defined as the 12 mutations with a frequency >10% across the pan-cancer cohort. There are 126 such interaction terms, excluding those linearly dependent on the other factors. These factors take in account cancer type-dependent contributions of mutations.

We applied the following filters to exclude factors from the initial design that may confound the regression:

At least 20 samples in the cohort belong to each factor (e.g. at least 20 samples belong to a certain cancer type);

Each factor has a maximum variance inflation factor (VIF) equal to 4, excluding interaction terms. This filter attempts to minimize collinearity, which may occur in this cohort due to cancer type-specific mutations (e.g. VHL in clear cell renal cell carcinoma). In this case, the gene expression signal cannot be properly factorized in the contribution of the collinear factors, and only the main factor will be retained (in our case the cancer type).

Using the same notation (where appropriate) as in voom, the GLM (1) is:

E(y_{g,i})=μ_{g,i}=xᵢ^{T}β_{g} (1)

where E( ) denotes an expected value of the variable within brakets, y_{g,i} is the log-counts per million (log-cpm) value for gene g in sample i, μ_{g,i} is the expected value, xᵢ is the vector of covariate values in sample i, β_{g} is the (unknown) vector of coefficients representing the contribution of each covariate on the expected value, and I_{g} is the explicitly formulated intercept of the GLM. In our formulation, the All model (2) becomes:

μ_{g,i}=I_{g}(Σₘ₌₁^{nCancerMutations}βₘxₘ+Σₜ₌₁^{nCancerTypes}βₜxₜ+Σ_{f=1}^{nTranscriptionFactors}β_{f}x_{f}+Σₗ₌₁^{nInteractions}βᵢxᵢ)^{T} (2)

where xₘ is a binary value {0,1} indicating the absence or presence of a mutation in gene m in the sample i; xₜ is a binary value {0,1} indicating the belonging of sample i to the cancer type t; x_{f} is a ternary value {−1,0,1} indicating whether the expression of transcription factor f in sample i is in the bottom quintile, 2^{nd} to 4^{th} quintile, or top quintile with respect to the distribution of its expression values in the pan-cancer cohort; and xₗ is a binary value {0,1} indicating whether there is the interaction/between the cancer type to which sample i belongs and a frequently mutated gene.

We excluded the following observations from this study:

All genes that have ambiguous annotation in Ensembl v73. This set corresponds to 565 genes.

All genes that were not detected in any sample. A gene is detected if at least 10 counts were reported in 10% of the samples. Although the opposite may occur due to an actual repression of the gene, this signal cannot be distinguished from genes that are misannotated or, more likely, from genes whose transcripts cannot be detected due to technical limitation in the sensitivity of the sequencing instrument. These observations do not add any information on the expression status of the (presumptive) gene and thus their removal will not alter the result of downstream analyses. This set corresponds to 1075 genes.

Overall, 1,575 genes were excluded from the initial set of 20,531 genes (65 overlapped between the above mentioned filtered sets), yielding a total of 18,956 genes analyzed.

Many factors in the All model are unlikely to contribute in explaining the expression of most genes, thereby increasing the risk of over-fitting. We adopted two different model selection methods to derive the most relevant factors while using a minimal number of factors. First, backward selection was used to exclude, at each iteration, the factor that is associated with the least number of differentially expressed genes. The procedure was stopped once the number of differentially expressed genes (defined as FDR<0.01 and |log₂FC|>1.5) was greater than 0.5% of all genes (i.e. 90 genes). The resulting GLM contains 38 factors (BS model). Second, we used L1-constrained regression shrinkage using the Lasso algorithm to compute, for each gene, the factors in the All model with a non-null coefficient. The penalty value used for the Lasso regression was calculated such that the mean 10-fold cross-validated error is minimum. The Lasso method was implented using the R-package glmnet (Friedman et al., 2010). We constructed a GLM based on the factors with a significant coefficient (|β|>log₂(1.5)) in at least 0.5%, of all genes (Lasso model), resulting in 29 factors. Finally, we constructed alternative GLMs that feature either only the cancer type (CT) or the transcription factor levels (TF) or the mutation statuses (Muts) or any other meaningful combination of these classes with interactions, if appropriate.

The best GLM was evaluated by first calculating the Bayesian information criterion (BIC) values for the goodness-of-fit of all genes by each GLM. This criterion was chosen for its ability to capture the trade-off between the goodness of fit and the stringent penalty on the number of factors utilized in the regression of the expression of a gene (for each GLM, there is a BIC value per gene), thus minimizing over-fitting. Given that the Lasso, BS, and onlyCT performed equally well, we compared the goodness-of-fit of these models in terms of Akaike information criterion (AIC) values, which, compared to BIC values, penalize a poorer goodness-of-fit over the number of factors. To this end, we computed, for each gene, the difference between the AIC value returned by the current GLM and the minimum AIC value observed using any of the three GLMs. From this, we calculated the AIC weight of the alternative GLMs in the regression of each gene. The AIC weights were transformed into probabilities that a certain GLM is the most likely to explain the expression of that gene. Finally, we counted for each GLM the number of genes whose expression is best explained by that GLM. If the onlyCT model is considered as a positive control for the regression of gene expression, the comparison of gene-wise BIC value between the onlyCT model and an alternative GLM was used to determine whether the additional factors in the alternative GLM provided a better goodness-of-fit while controlling for over-fitting (a positive comparison means that the gene-wise BIC values are skewed towards more negative values when using the alternative GLM). The model selection was implemented in R 3.1.2.

### Gene-Set Analyses

The gene-set analyses were performed using the R-package Piano (Varemo et al., 2013). In all analyses, we evaluated the significance of a gene-set using the genes found here to be associated with a mutated gene (here on mutation-associated genes). For each mutated gene, the list of mutation-associated genes is generated using the differentially gene expression analysis based on the BS model (see Differential gene expression analysis). In the case of enrichment of the 189 gene-sets representing each a genetic perturbation in a key cancer-associated gene [retrieved from the Molecular Signatures Database (MSigDB)], the significance of a gene-set was tested using the Stouffer's test, and the p-values were controlled for multiple testing by transformation to FDR using the Benjamini and Hochberg correction. To check for consistency between the genetic perturbation represented by a gene-set and the expected effect on gene expression by a mutation, we compared separately the gene-sets (if significant, i.e. gene-set FDR<0.01) mostly associated with up-regulated or down-regulated genes (in Piano, so called “mixed directional” classes). For example, genes here found up-regulated when CTNNB1 (β-catenin) is mutated are significantly associated with the BCAT_BILD_ET_AL_UP gene-set, in which β-catenin (BOAT) was over-expressed in primary epithelial breast cancer cell.

In the case of enrichment of GO biological processes, 8255 gene-sets were retrieved using the R-package biomaRt (Durinck et al., 2009). The significance of a gene-set was tested using the consensus between six tests (Fisher's test, Stouffer's test, Reporter test, Tail strength test, mean, and median), and the p-values were controlled for multiple testing by transformation to FDR using the Benjamini and Hochberg correction. If gene-set FDR<0.01, the underlying biological process is deemed significantly associated with the mutated gene. To compute the probability that multiple mutated genes are simultaneously associated with a gene-set, we designed a permutation test in which the gene-sets significantly associated with a mutated gene are randomly permuted 10,000 times. Then, we calculated a p-value as the frequency at which a gene-set is randomly associated with a number of mutated genes greater or equal to that observed prior randomization. Next, we computed using the Fisher′ Exact Test which ancestor GO category (defined as the children of the GO term biological process) were overrepresented by the GO terms that showed significant convergence. Finally, we estimated the robustness of the supposed overrepresentation of an ancestor GO category repeating this above operation using only those GO terms that showed convergence by an increasing number of mutated genes (i.e. given n GO terms associated with at least x mutated genes, we computed which GO ancestors are overrepresented by the n GO terms).

### Extraction of the High-Convergence Reaction Sub-Network

The human genome-scale metabolic model HMR2 was downloaded from http://www.metabolicatlas.com/. We generated a reaction network from the model where reactions are nodes, and an edge links two nodes if there is at least one metabolite shared by the two reactions. We excluded 18 metabolites with exceptionally high degree (>200) to prevent a combinatorial explosion of reaction-reaction edges. Then, we used the jActiveNetwork algorithm (Ideker et al., 2002) to extract from this reaction network a connected sub-network that maximizes the number of mutations converging to it. To this end, we counted for each reaction the number of times that any mutation is found associated with a gene encoding that reaction. Each reaction of the network was then scored using this count. We subtracted a penalty equal to 5 to the score to ensure that the extracted sub-network was reasonably small yet comprised as many reactions with at least four mutated genes converging to them. This prevented that biologically related mutated genes (like KEAP1 and NFE2L2) could significantly bias the emerging sub-network. Artificial reactions introduced in HMR2 for modeling purposes (defined by the HMR2 sub-systems Isolated, Artificial reactions, Exchange reactions, Pool reactions) were further penalized with a score of −100. The search was implemented using the R-package BioNet (Beisser et al., 2010). The returned high-convergence reaction sub-network contained 90 reactions (nodes) out of the 8184 reactions that were present in the reaction network.

### Analysis of the High-Convergence Reaction Sub-Network

We characterized the high-convergence reaction sub-network by comparing the frequency of metabolites and pathways represented by the reactions in the sub-network to the background frequency in HMR2. The overrepresentation of metabolites and pathways was calculated using the Fisher's Exact Test. To further aid the interpretation of the reactions part of the high-convergence reaction sub-network, this was broken down in reaction clusters, defined as sets of reactions that share the same gene-reaction association. These are returned by applying unsupervised hierarchical clustering to the gene-reaction association matrix in HMR2 limited to include the reactions in the high-convergence reaction sub-network and the genes associated with at least one mutated gene. This operation reduced the complexity of the high-convergence reaction sub-network to 14 reaction clusters.

### Curation of the High-Convergence Reaction Sub-Network

Starting from the above analysis, we consulted the literature to frame the high-convergence reaction sub-network in the context of well-defined metabolic functions and reconstruct a comprehensive pathway. Also, we manually reviewed every metabolic gene associated with at least one mutated gene and verified if there exist a relation with the emerging pathway. We discarded a candidate gene if its pan-cancer expression level was not appreciable in a reasonable number of samples (minimum library size-adjusted log-cpm in the top 20% equal to 1).

We initially focused on arachidonic acid and its metabolism given its prominent enrichment in the high-convergence reaction sub-network compared to HMR2. The reaction clusters #3 and #4 indicate inclusion of reactions belonging to the cytochrome P450-pathways of arachidonic acid. These include reactions in the hydroxylase pathway, catalyzed by CYP4F11. Other mutation-associated genes belong to the epoxygenase pathway, specifically CYP2S1. CYP4X1 is also a likely member of this pathway, but evidence for specificity to arachidonic acid is still inconclusive. The reaction clusters #5 and #7 implicate another major route of arachidonic acid, the cyclooxygenase (COX) pathway to produce prostaglandins. In total, 8 mutation-associated genes in the metabolism of prostaglandin H₂, the first product of arachidonic acid conversion in the COX pathway. Among these is PTGS1 (also known as COX-1), which catalyzes the first common step in the COX pathway from arachidonic acid to prostaglandin H₂. PTGES, GSTM2 and GSTM3 can convert prostaglandin H₂ to prostaglandin E₂, which in turn can be converted to prostaglandin F₂₋ₐₗₚₕₐ by CBR1. HPGDS is responsible for the conversion of prostaglandin H₂ to prostaglandin D₂. AKR1C3 can reduce prostaglandin H₂ and D₂ to prostaglandin F₂₋ₐₗₚₕₐ and 11-beta-prostaglandin F₂₋ₐₗₚₕₐ, respectively. Finally, HPGD inactivates prostaglandin D₂, E₂, and F₂₋ₐₗₚₕₐ by conversion to their respective dehydrogenated forms. The third pathway of arachidonic acid metabolism is the lipoxygenase (LOX) pathway. Manual review of mutation-associated genes revealed that four genes encode for reactions downstream of arachidonic acid. On one hand, three genes are involved in the metabolism of two compounds derived from leukotriene A₄, which is itself derived from arachidonic acid, namely leukotriene B₄ and C₄. CYP4F3 and PTGR1 catalyze the inactivation of leukotriene B₄ either by ω-oxidation or via the 12HDH/15oPGR pathway respectively. GGT6 is involved in the conversion of leukotriene C₄ to leukotriene D₄. On the other hand, one gene, ALOX15, catalyzes the direct synthesis from arachidonic acid of yet another class of LOX products, lipoxilins. The reaction cluster #10 implicates reactions upstream of arachidonic acid. Manual review revealed a significant number of enzymes responsible for the cleavage of arachidonic acid from cellular lipids among the mutation-associated genes. PLA2G2A, PLA2G4A, PLA2G4E, and PLA2G10 all belong to the class of phospholipases A₂ and function to release free fatty acids from the sn-2 position of phospholipids. Noteworthy, PLA2G2A shows an exquisite preference towards phospholipids containing arachidonic acid at the sn-2 position. FAAH2 also affects arachidonic acid availability. Specifically, FAAH2 degrades endogenous cannabinoid anandamide to release arachidonic acid. Finally, ELOVL2 elongates selectively activated arachidonic acid and MBOAT2 is involved in the Land's cycle to reincorporate activated arachidonic acid in the membrane lipids.

Next we focused on xenobiotics metabolism, among the most enriched pathways in the high-convergence reaction sub-network. We first noticed that four genes overlap with the metabolism of arachidonic acid. AKR1C3, CBR1, GSTM2 and GSTM3 have also reported activity in the detoxification of electrophilic xenobiotics. Reaction clusters #2, #9, and #14 implicate phase I of xenobiotics metabolism (also called functionalization). After manual review, we gathered a total of 22 genes involved in the functionalization phase. The great majority (20) are oxidoreductases in the family of cytochrome P450 (CYP3A5), alcohol dehydrogenases (ADH1C, ADH6, ADH7, ADHFE1), flavin-containing monoxygenases (FMO3, FMO4, FMO5), aldo-keto reductases (AKR1B10, AKR1B15, AKR1C1, AKR1C2), quinone reductases (NQO1, NQO2), carbonyl reductases (CBR3), aldehyde dehydrogenases (ALDH3A1, ALDH3A2, ALDH3B1), and amine oxidases (AOC1, MAOB). The two remaining genes, CES1 and EPHX1, belong instead to the class of hydrolases. Reaction cluster #1 implicate phase II of xenobiotics metabolism, also known as conjugation. Collectively, we found 10 genes that can catalyze conjugation reactions among the mutation-associated genes. UGT1A1, and UGT1A6 are UDPGA transferases that carry glucuronidation reactions on xenobiotics. GSTA2, GSTM1, GSTM4, and MGST1 catalyze the conjugation of glutathione. SULT1A1, SULT1A2, and SULT1A4 belong to the family of sulfotransferases and are responsible for sulfonation reactions on xenobiotics using PAPS as cofactor. ACSL5 is a acyl-CoA synthetase that conjugates xenobiotic carboxylic acid by forming acyl-CoA thioesters.

Finally, we also observed five transporters for both arachidonic acid-derived products and solubilized xenobiotics in the list of mutation-associated genes. The organic anion transporters SLCO2A1 and SLCO1B3 show affinity for prostaglandin D₂ and leukotriene C₄, respectively. The ABC transporters ABCC1, ABCC2 and ABCC3 are renowned for their ability to move a variety of xenobiotics, but other substrates include prostaglandin A₁, A₂, D₂, E₂, 15d J₂ and leukotriene C₄.

The enrichment for the occurrence of oxygen- and glutathione-consuming reactions in the high-convergence reaction sub-network persuaded us to investigate which other genes support their metabolism. Reaction clusters #6 and #13 feature two genes in glutathione metabolism, GPX2 and GPX3. In addition, there are four more enzymes among the mutation-associated genes that are involved in glutathione biosynthesis, GCLC, GCLM, GSR, and OPLAH. These expand the list of glutathione-utilizing enzymes in the candidate pathway to a total of 15 members. In addition, several mutation-associated genes encode for reactions that use oxygen, most notably 7 members of the cytochrome P450 (CYP2W1, CYP4B1, CYP4X1, CYP24A1, CYP27A1, CYP27B1, CYP39A1) and 4 others associated with at least two mutations: HGD participate in the metabolism of tyrosine; CDO1 catabolizes cysteine and controls its cellular concentration; CP is a glycoprotein involved in iron ion homeostasis; and MOXD1 is a monooxygenase of unknown substrate. These expand the list of oxygen-utilizing reactions in the candidate pathway to a total of 21 members.

We neglected the result on the enrichment for the estrogen metabolism pathway because the associated genes are best explained by xenobiotics metabolism.

During the validation of our findings (see below), the increased statistical power allowed us to discover 9 new mutation-associated genes that encode for reaction in or related to AraX. Six of these genes belong to arachidonic acid metabolism: ALOX5 and LTC4S belongs to the LOX pathway; CYP2E1 belongs to the epoxygenase branch of the cytochrome P450-pathway; PLA2G6 and PLA2G12A are phospholipases A₂ involved in the release of arachidonic acid from the plasma membrane; and PTGS2 encodes for the first step in the conversion of arachidonic acid to prostaglandins together with PTGS1. The remaining three belong to xenobiotics metabolism: FMO1 is a flavin-containing monoxygenase in the functionalization phase thioesters, while GSTO1 and GSTO2 belong to the conjugation phase.

The so-reconstructed candidate pathway features 27 genes attributable to arachidonic acid metabolism, 35 genes attributable to xenobiotics metabolism, 17 genes that mediate glutathione and oxygen metabolism, and 5 genes in the transport system. We reviewed each protein in this pathway in UniProt and/or Reactome to validate the gene annotation provided by literature. In total, 84 metabolic genes are represented in this pathway. We termed this pathway AraX.

### Enrichment of Pathways by Mutation-Associated Genes

We calculated the overrepresentation of AraX by each group of mutation-associated genes compared to any other KEGG pathway (186) or Reactome pathway (674), as retrieved in MSigDB, using the Fisher's Exact Test. The mean enrichment of a pathway across all mutations was subject to bootstrapping (10,000 replicates) in order to calculate the 95% confidence interval for the mean enrichment. This operation allows evaluating the robustness of a pathway mean enrichment to outliers (i.e. mutated genes strongly associated with a pathway).

### Validation of the Generalized Linear Model and Mutation-Associated Genes

We performed differential gene expression analysis, as described above, using the BS model on the validation cohort, consisting of 4,462 samples. The samples encompassed the same 13 cancer types as in the discovery cohort (range: 94-978 samples). We verified that the factors in the BS model featured at least 20 samples also in the validation cohort. As described above, the comparison of gene-wise BIC value between the onlyCT model and the BS model was used to determine whether the additional factors in the BS model provided a better goodness-of-fit also in the validation cohort. We sought to validate the list of genes associated with a mutated gene in the discovery cohort and their corresponding fold-changes by linearly correlating them to the fold-changes estimated using the BS model on the validation cohort. Finally, to prove that the expression changes associated with multiple mutated genes in the validation cohort indeed converge in the deregulation of AraX, we computed the over-representation of this pathway compared to any other KEGG or Reactome pathway as described earlier (see Enrichment of pathways by mutation-associated genes).

### Survival Analysis

The deregulation at the level of gene expression for a metabolic pathway in a sample was estimated using Pathifier (Drier et al., 2013). This algorithm returns a score between 0 and 1 that represents the extent to which the expression of a pathway in a sample is deviating from the centroid pathway expression in normal samples. Hence, we calculated the score for all tumor samples in this study belonging to six cancer types for which matched normal samples were available in TOGA. These cancer types were breast invasive carcinoma, colon adenocarcinoma, head and neck squamous cell carcinoma, lung adenocarcinoma, lung squamous cell carcinoma, and uterine corpus endometrial carcinoma. The normal samples were used to provide the reference expression level of the pathway in a tissue.

We regressed the survival time until censoring or death to the AraX deregulation score for each sample in the discovery cohort (718 samples) to estimate whether AraX deregulation conferred a selective advantage to cancer evolution. We adopted as controls the same regression to the deregulation scores for other KEGG metabolic pathways (70) or for a gene-set including 3714 metabolic genes (ALLM). Then, we used a multivariate lasso penalized Cox regression model to calculate which metabolic pathway deregulation has the foremost effect in the prediction of survival, using as variables the deregulation score of the 70 KEGG metabolic pathways, AraX, and ALLM. The selection of variables relevant to predict survival was performed using increasing values for the lasso penalty (log-λ) used in the regression. The optimal penalty value was calculated such that the mean 10-fold cross-validated error was minimum. Out of 72 initial variables, only 3 variables were predictive of survival at the optimal penalty. To further rule out that a simple pathway deregulation is sufficient to predict poor prognosis, we performed univariate Cox regression of survival on the deregulation scores of any KEGG pathway (also non metabolic ones) with more than 100 genes and compared the Wald test statistic and log-hazard ratio per unit of deregulation score to the regression on AraX deregulation scores.

We determined whether poor prognosis could be predicted by the level of deregulation of AraX by equally splitting all samples in this study belonging to the six cancer types used above and with complete survival information (1,908 samples) into a discovery and validation sub-cohorts and stratifying the samples into low or high deregulation. The threshold score upon which a sample is classified as highly deregulated was computed in the discovery sub-cohort by using maximally selected rank statistics, which identifies a threshold score that maximizes the difference in survival between the two groups and tests its statistical significance. A robust threshold score was finally selected by repeating this computation using 1,000 bootstraps. Kaplan-Meier curves were generated for the two groups, and the significance of survival difference was estimated using the Wald test. The validity of the threshold score and the difference in survival between the two groups were verified in the validation sub-cohort. The difference in survival according to the low vs. high stratification was finally computed using the Wald test leveraging on all samples, and the corresponding statistic was tested against sub-sampling using 10,000 bootstraps and random sample label permutation using 10,000 permutations.

Due to missing clinical information in a non-negligible number of samples, we verified the independency of AraX deregulation from other prognostic clinical features individually, by performing a statistical test of dependency in the subset of samples where the information was reported. We tested a correlation between low vs. high AraX deregulation and age using the Wilcoxon rank-sum test in 1,343 samples and with metastatic status using the Fisher exact test in 351 samples. We tested an association between the AraX deregulation scores and the tumor stages within each cancer type using the likelihood ratio test, in a number of samples ranging 48 to 132 depending on the cancer type (endometrial cancer was excluded because no samples were annotated with tumor stage information). We tested whether the distribution of AraX deregulation scores are cancer type dependent using a likelihood ratio test. The correlation between the cancer type-specific distribution of AraX deregulation scores and the 5-year survival for cancers of the corresponding tissue (as retrieved from https://nccd.cdc.gov/uscs/Survival/Relative_Survival_Tables.pdf) was tested using a likelihood ratio test. The significance of the univariate regression of survival in a given cancer type and low vs. high AraX deregulation (according to the threshold score identified earlier in the pan-cancer cohort) was tested using the Wald test. A power analysis for this test at a confidence level (α=0.01 was conducted by sub-sampling the pan-cancer cohort into sizes ranging 100 to 1,900 samples 1,000 times, and by counting the percent of times a significant association between survival and AraX deregulation was found.

### Results

Identification of the Factors that Correlate with Gene Expression Changes in Cancer Using Generalized Linear Models

We first sought to test the existence of a statistical association between gene expression changes and the presence of a mutation in a cancer-associated gene in the tumor, i.e. if occurrence of a mutation correlates with an increase or decrease in the mRNA level of a gene. RNA-seq profiles for 1,082 primary tumor samples were retrieved from The Cancer Genome Atlas for 13 distinct cancer types (range of 21-199 samples per type) for which a validated mutation spectrum was available (FIG. 1A). In this cohort, we focused on the 158 genes mutated at moderate frequency (>2% samples), of which 12 are mutated at high frequency (>10% samples). We hypothesized that the level of gene expression could be factorized as the contribution of four sample features: the histopathological cancer type; the expression level of transcription factors; the presence or absence of a mutated gene; and the synergy induced by occurrence of a mutated gene in a particular cancer type. We therefore employed the established statistical framework of generalized linear models (GLM) to perform a linear regression of gene expression on the following factors: the 13 cancer types (CT); the activation status of 119 well-characterized transcription factors (TFs); the presence or absence of a mutation in one of the 158 genes mutated at moderate frequency (Muts); and the interaction terms between the presence of a high frequency mutated gene and the cancer type where it occurred (lnts) (FIG. 1B). This generated an initial GLM (All), which comprised 316 non-collinear factors, with at least 20 samples per factor.

Likely, many of these factors do not contribute significantly to explain the expression level of a gene. Hence, we employed different methods for model selection, including backward selection and regularized regression via the Lasso algorithm. These methods identify a minimal number of relevant factors while maintaining an acceptable prediction of the observed gene expression levels. Each method returned a set of relevant factors that constitute an alternative GLM to the initial All model (FIG. 1B). In total, we generated 11 GLMs: a backward selection (BS) model (yielding 38 factors); a Lasso model (Lasso, 29 factors); and 9 models solely based on a subset of the sample features (i.e. only CT, or only TFs, or only Muts, or any other combination of these). The best GLM was selected based on the goodness-of-fit between observed and predicted expression level for each gene and the number of factors on which the GLM leverages. A quality measure of this trade-off is the Bayesian information criterion (BIC), which tends to penalize models with too many factors (i.e. higher BIC values), thereby reducing over-fitting. Using each GLM, we calculated the BIC values for each gene (FIG. 2A). The Lasso, BS, and onlyCT models performed equally well compared to any of the other GLMs (FIG. 2A). To choose among these three GLMs, we resorted to calculate also the Akaike information criterion (AIC), which tends to penalize models with poorer goodness-of-fit. The conditional probability that a particular GLM performs better in the prediction of the expression level of a given gene can be derived by directly comparing the AIC values of the three GLMs in the form of AIC weights. This analysis revealed that in the case of 15,040 genes (79%), the BS model has the highest probability of predicting the expression more accurately than the Lasso model and the GLM in which only cancer type factors were used (onlyCT) (FIG. 2B). We noticed that the cancer type yet represents the strongest factor in the prediction of gene expression changes, as exemplified by the reasonable goodness-of-fit achieved by the onlyCT model (FIG. 2A). A comparison of the gene-wise BIC value using either the onlyCT model or the BS model revealed a shift towards lower BIC values when employing the BS model, suggesting that the additional factors in the BS model contribute to the expression level of many genes (FIG. 2C). Overall, the goodness-of-fit between observed vs. predicted gene expression levels across all 1,082 samples using the BS model generated a Pearson correlation coefficient R=0.963 (FIG. 2D). Considering these results, we adopted the BS model to test for associations between gene expression and cancer mutations.

Derivation of Gene Expression Changes Associated with Cancer Mutations

Since factors other than the cancer type contributed to the observed gene expression level, we investigated whether mutations in cancer-associated gene are among these (FIG. 1D). Interestingly, 9 mutated genes (out of the initial 158 genes) featured as factors in the BS model. These mutated genes (here on also simply referred to as mutations) are CTNNB1 (also known as β-catenin), IDH1, KEAP1, NFE2L2 (Nrf2), NSD1, PTEN, RB1, STK11 (LKB1), and TP53. The second best performing GLM, the Lasso model, also featured 6 mutations as factors, all of which are among the 9 mutations identified by the BS model. The contribution of each mutation to gene expression was independent of cancer type and the activation of a given transcription factor, as these contributions are already accounted for by their respective factors. Thus, we sought to derive which genes change expression in association with the occurrence of each of these mutations in the tumor. To this end, we tested the significance of each association by drawing from a RNA-seq adapted differential gene expression analysis, performed using voom (Law et al., 2014) (FIG. 1E). At a false discovery rate (FDR)<1% and minimum absolute fold-change >50%, we found that on average the occurrence of a mutation correlated with expression changes in 495 genes (range of 302-764 genes per mutation), for a total of 2,750 genes [note that 1,075 genes (39%) were associated with more than one mutation].

We sought to validate whether the genes found here to be associated with one of the 9 mutations changed their expression in independent experiments. To this end we used 189 experimentally derived gene-sets, each representing genes whose expression is altered in response to perturbation in a key cancer-associated gene. We then performed a gene-set analysis for each mutation in order to evaluate if the genes found to be associated with it are enriched in any of these 189 gene-sets. We observed an overall high consistency between the direction of regulation of the genes found here to be associated with a given mutation and corresponding experimentally derived gene-sets (FIG. 8). For example, genes here found to be up-regulated when RB1 is mutated significantly enriched the RB_P107_DN.V1_UP gene-set, which features genes up-regulated in primary keratinocytes from RB1 and RBL1 skin specific knockout mice; genes here associated with NFE2L2 mutations are exquisitely over-represented in the NFE2L2.V2 gene-set, which contains genes up-regulated in embryonic fibroblasts with knockout of NFE2L2; or genes here found to be up-regulated in occurrence to CTNNB1 mutations specifically enrich the BCAT_GDS748_UP gene-set, which includes genes up-regulated in kidney fibroblasts expressing constitutively active form of CTNNB1.

Taken together, these results suggest that differential gene expression analysis based on the BS model uncovered associations between gene expression and the 9 mutated genes that recapitulate correlations observed experimentally. These expression changes are likely to be context-independent, not attributable to a specific cancer type.

### Convergence of Mutation-Associated Gene Expression Changes in the Regulation of Metabolism

Next, we were interested in elucidating if the genes associated with each mutation are involved in specific biological processes. In particular, we expected that the 9 mutations associate independently with processes linked to important cancer-relevant phenotypes, known as the hallmarks of cancer. Convergence on any of these processes would provide strong evidence that cancer mutations drive the selection of clones that feature properties reflecting these hallmarks. Hence, we checked if the genes associated with the 9 mutations are enriched in any particular biological process, each represented by a distinct Gene Ontology (GO) term. We employed consensus gene-set analysis using Piano (Varemo et al., 2013), which revealed a diverse number of GO biological processes that are significantly associated with each of the examined mutations (FDR<0.01). However, contrary to the premises, only a small number of GO biological processes simultaneously associated with more than one mutation (FIG. 3). We further classified the processes that displayed a significant convergence compared to 10,000 random permutations (P<0.01) according to the 24 ancestor categories they are assigned to within the GO hierarchy. Hereby we observed an over-representation of the GO ancestor category of metabolic processes. Intriguingly, metabolism is the GO ancestor category with the most stable overrepresentation when more stringent criteria for convergence are enforced. Collectively, these results suggest that the presence of each of these 9 mutations entails a diverse spectrum of gene expression changes in terms of affected biological processes, but that the reprogramming induced by these mutations primarily converges on regulation of metabolism.

### Mutation-Associated Gene Expression Changes Converge on a Sub-Network of Metabolic Reactions

Metabolism appeared to be the biological process that displayed the largest extent of regulation associated with the 9 mutations. Indeed, mutations in cancer genes have been recognized to regulate metabolism to meet the metabolic requirements of rapid proliferation and allow cancer cells to adapt to the microenvironment. Others and we have previously found that distinct cancer types featured few common gene expression changes in metabolism during the transformation, primarily ascribed to altered nucleotide biosynthesis. However, these studies could not distinguish whether the observed changes are attributable to a common adaptation process during cancer progression or are rather the consequence of a specific mutation event. To interrogate this, we selected among the genes here associated with 9 mutations those that overlapped with the 3765 genes that participate in the human metabolic network. This set corresponds to 499 metabolic genes, each associated with the presence of at least one of the 9 mutations, for a total of 852 associations.

The network of associations between a mutation and regulated metabolic genes revealed a number of genes on which multiple mutations converge. However, no metabolic gene showed convergent association with all mutations, nor was there a canonical metabolic process to which all mutations are associated (FIG. 3). We therefore tested the hypothesis that mutations collectively associate with metabolic genes encoding for a common yet non-canonical sub-network of reactions. We first mapped for each reaction in the human metabolic reaction network the number of mutations that converge on it, through the association with the underlying reaction-coding gene(s) (FIG. 4A). This highlighted distinct clusters of reactions within the metabolic network. To extract the largest functional cluster, we searched for a connected sub-network of reactions in which the number of converging mutations is maximized by using the jActiveNetworks algorithm (Ideker et al., 2002). This approach returned a single high-convergence reaction sub-network (FIG. 4B). We characterized this sub-network by determining whether its nodes significantly enrich any pathway and/or metabolite compared to the background human metabolic network. We uncovered that the sub-network featured an over-representation of the metabolism of xenobiotics, estrogen, and arachidonic acid (FIG. 4C). In addition, individual metabolites such as hydrochloride (a by-product of xenobiotics metabolism), glutathione, arachidonic acid, and oxygen were also over-represented within the sub-network (FIG. 4D). Collectively, these findings suggest that regulation of a sub-network of reactions that connects arachidonic acid and xenobiotics via glutathione and oxygen correlates independently with 9 frequently mutated genes in cancer.

Curation of the high-convergence sub-network of metabolic reactions: AraX

Starting from the high-convergence reaction sub-network, we manually curated a representation of the candidate pathway that best represents these reactions according to the literature. We termed this pathway AraX (FIG. 5), for arachidonic acid and xenobiotic metabolism. The AraX pathway contains 20% of all mutation-metabolic gene associations uncovered above (166 of 852). One branch of the AraX pathway comprises reactions that control the availability of arachidonic acid and catalyze its conversion to eicosanoids. The second branch facilitates the detoxification of xenobiotics. Importantly, seven enzymes encoded by the genes associated with this pathway are involved in both branches (e.g. CYP4F11). In addition, there are transporters that can secrete the end products of the pathway (FIG. 5). The main co-substrates for arachidonic acid and xenobiotic metabolism are oxygen and glutathione, whose levels are controlled by the remaining genes in the pathway.

The overrepresentation of xenobiotics metabolism with cancer mutations was unexpected, considering that the samples used for this study were derived from untreated tumors. The importance of AraX in cancer may reside in its individual components. Aberrant arachidonic acid metabolism regulates processes critical for cancer progression, mainly by establishing a tumor-supporting microenvironment where immune cells and endothelial cells are recruited to produce mitogens, pro-inflammatory cytokines, and angiogenic factors. Enzymes within the xenobiotics metabolism form reactive intermediates from exogenous and endogenous substrates that can cause cancer initiation, potentially by promoting genotoxicity. Both pathways are a primary source of cytosolic reactive oxygen species, which exhibit a characteristically abnormal concentration in many types of cancer cells. Finally, a number of xenobiotic-metabolizing enzymes and transporters in AraX confer cancer cells with mechanisms of detoxification and drug-resistance. Taken together, this suggests that AraX is implicated in a number of host-cancer interactions that result in pro-tumorigenic functions.

We confirmed that compared to all 186 KEGG pathways AraX is, on average, the most significantly enriched pathway by the genes associated with a mutation (odds ratio, 17.07; 95% 10,000 bootstraps confidence interval [CI], 4.62 to 26.70); FIG. 5B), followed by xenobiotics metabolism by cytochrome P450 (odds ratio, 5.91; 95% CI, 1.73 to 9.44). Similar results were obtained when AraX was compared to the 674 Reactome pathways (FIG. 5C). Noteworthy, these KEGG and Reactome pathways also include signaling pathways dysregulated in cancer and that include non metabolic genes, contrary to AraX, which was solely constructed based on metabolic genes. Overall, this finding suggests that regulation of a network of metabolic reactions connected to arachidonic acid and xenobiotics metabolism and mediated by glutathione and oxygen is advantageous in cancer, since 9 frequently mutated genes independently entail transcriptional changes that converge on this pathway.

### Validation of Convergence on AraX Regulation in an Independent Cohort

We sought to validate whether the expression changes here correlated with the occurrence of cancer mutations were reproducible in an independent cohort and, in particular, if these correlations indeed converged primarily in the regulation of AraX. We retrieved genomic and transcriptomic data from 4,462 primary tumor samples spanning the same 13 cancer types (range 94-978 per type). This validation cohort consisted of samples made available by The Cancer Genome Atlas during the period this study was being conducted. First, we verified whether the BS model was over-fitted to the samples in the discovery. We compared the BIC values in the regression of the expression of each gene by using either the BS model or the onlyCT model. The BS model outperformed the onlyCT model in the prediction of expression of most genes, as proved by a substantial shift towards lower BIC values (FIG. 6A). This suggests not only that additional factors other than the cancer type are important to explain the expression level of many genes, but also that those factors previously included the BS model provide a noticeable contribution. In particular, we checked whether gene expression changes that we associated to the presence of a mutation in the discovery cohort were consistent with the changes associated to a mutation in the validation cohort (FDR<1% and minimum absolute fold-change >50%). In the validation cohort, the occurrence of a mutation correlated on average with expression changes in 796 genes (range 169-2,235 per mutation), for a total of 4,810 genes [note that 1,455 genes (30%) were associated with more than one mutation]. For each of the 9 mutated genes, we found highly significant linear correlations between expression fold-changes of associated genes estimated using either the discovery or the validation cohort, with Pearson correlation coefficients ranging 0.26 for CTNNB1 to 0.66 for NFE2L2 (P=5·10⁻³⁴ to 7·10⁻²⁹⁷, FIG. 6B).

Next, we verified whether expression changes correlated to each of the mutation in the validation cohort converged preferably on AraX rather than on any other metabolic process, as suggested by our previous results. Compared to KEGG and Reactome pathways, AraX is, on average, the second most significantly overrepresented pathway (odds ratio, 6.98; 95% bootstrap CI, 2.95 to 13.24); FIGS. 6C-D), and the only pathway where we observed a consistent overrepresentation by all 9 mutated genes. Noteworthy, only 12 of 4,810 genes were associated to at least 6 mutated genes in the validation cohort, and three of these belonged to AraX (HGD, ADH7, and ALDH3A1). Consistently, multiple mutations converged in the association with these three genes already in the discovery cohort. The increased statistical power in the validation cohort allowed us to discover 9 new mutation-associated genes that encode for reactions in or related to AraX, like PTGS2 (also known as COX-2) or FMO1. With these additions, the AraX pathway is encoded by 84 genes (FIG. 5). Taken together, these findings indicate that our analysis yielded reproducible correlations between gene expression and occurrence of a cancer mutation. Importantly, these correlations primarily converged on the regulation of AraX over any other metabolic process here considered.

### Deregulation of AraX in Cancer is the Strongest Predictor of Survival Among Metabolic Pathways

We sought to investigate the implication of the convergence on AraX in cancer. We observed no obvious pattern in the direction of the regulation of AraX by the different mutations, even though we noticed similar effects on AraX in case of mutated KEAP1, NFE2L2, STK11, and PTEN, which tended to be opposite in case of mutated CTNNB1, IDH1, NSD1, RB1, and TP53 (Table B). Nevertheless, there was an evident mutation-specific modulation in the expression of AraX genes, with varying degrees of overlap. This poses a challenge when devising an intervention strategy to normalize the expression or activity of the AraX pathway aimed at halting cancer progression. On the other hand, this also suggests that a generic deviance (i.e. deregulation) in the expression of AraX is likely to confer a context-independent selective advantage in cancer. Therefore we speculated that the extent of AraX deregulation in the tumor should be predictive of an independent measure of selective advantage, for example patient's survival. Hence, we first estimated a deregulation score for the AraX pathway in each tumor sample using Pathifier (Drier et al., 2013). This score captures the extent to which the expression of a pathway in a tumor sample deviates from its expression in the normal tissue of origin. Then, we performed survival analysis for a subset of the discovery cohort consisting of 718 samples, so selected because they encompassed 6 cancer types for which reference normal samples were available. We regressed the survival time on tumors' AraX deregulation score using a Cox proportional hazards model, and we observed a significant increase in hazard with higher AraX deregulation (p=6·10⁻⁸). We tested whether a similar trend could be observed concomitantly with high deregulation of any other metabolic pathway or metabolism in general. However, compared to the 70 KEGG metabolic pathways and a gene-set comprising 3,714 metabolic genes, the deregulation of AraX ranks as the best and most robust predictor for survival as estimated by a Lasso penalized Cox proportional hazard model (FIG. 7A). At the cross-validated penalty value (log-λ=−2.5), only two other KEGG metabolic pathways are predictive of survival, oxidative phosphorylation and the pentose phosphate pathway. Nevertheless, AraX deregulation score resulted in the highest hazard (log-hazard ratio per unit of deregulation score, 0.30, FIG. 7A). This result suggests that AraX deregulation is predictive of survival likely because it confers an evolutionary advantage, and not due to a general deregulation attributable to an advanced tumor stage. To further corroborate this, we could not achieve a comparably significant increase in hazard when we performed an univariate Cox regression of survival on the deregulation score of pathways larger than AraX like purine metabolism (159 genes) or cell cycle (128 genes), despite their established role in malignant transformation (FIG. 7B-C).

We investigated whether poor prognosis can be attributed to the fact that advanced tumors select for clones with high AraX deregulation via mutagenesis by stratifying patients into low vs. high deregulation. To this end, we gathered a subset of samples from both cohorts consisting in 1,908 samples, so selected to represent the same 6 cancer types as above (range 184-778 per type), and randomly split them in two sub-cohorts (954 sample each).

Then we first verified if there was an optimal threshold score for AraX deregulation that maximized the difference in prognosis between patients in the discovery sub-cohort using maximally selected rank statistics. This returned a statistically significant threshold score for AraX deregulation equal to 0.764 (p=7·10⁻³, 1,000 bootstraps 95% CI: 0.731-0.802), above which patients have indeed substantially worse clinical outcome (log-rank test p=8·10⁻⁶ FIG. 7D). This correlation was independently confirmed when we applied the threshold to classify samples in the validation sub-cohort as either “low” or “high” Arax deregulation (p=1·10⁻⁵, FIG. 7E). When leveraging on all samples, there was an evident correlation between sample classification into “low” versus “high” AraX deregulation and survival (Wald test p=6·10⁻¹⁰). The increased hazard was robust to sub-sampling (hazard ratio=2.26, 10,000 bootstraps 95% CI: 1.72-2.93) nor it was attributable to a bias in the score distribution, as verified by randomly shuffling the sample labels 10,000 times (permutation test p<10⁻⁵).

Finally, we sought to characterize the prognostic relevance of AraX deregulation. We did not detect any dependency between the low or high AraX deregulation and other relevant clinical features, in that we found no correlation with age (Wilcoxon rank-sum test p=0.745), with metastatic status (Fisher exact test p=0.199) nor, within each cancer type, the scores associated with the tumor stage (likelihood ratio test p=0.488 to 0.782, excluding endometrial cancer for missing information). We observed an association between a cancer type and its AraX deregulation score (ranging 0.28 in endometrial cancer to 0.67 in head and neck squamous cell carcinoma, likelihood ratio test p<10⁻¹⁶), even though within each cancer type the samples can span a large range of scores (FIG. 9A). The AraX deregulation scores for each cancer type display a low inverse correlation with the corresponding 5-year survival for cancers of the same tissue (likelihood ratio test p<4·10⁻¹², FIG. 9B), which is suggestive that more aggressive cancer types tend to feature higher AraX deregulation. We were not able to decisively separate the two effects on survival because an analysis of statistical power indicated that at least 1,220 samples were needed to have a >50% chance to detect a significant association at our confidence level (α=0.01). Nevertheless, in the case of the cancer type with most samples, breast invasive carcinoma (N=778), we recovered a positive trend between AraX deregulation and survival (age-adjusted hazard ratio=3.468, 95% CI: 1.03-11.7, p=0.044).

Overall, the strong association of AraX deregulation with poor prognosis underscores the biological significance of this pathway in cancer, and suggests that aberrant expression of AraX confers a selective advantage for cancer progression more than for any other metabolic processes.

### DISCUSSION

Cancer cells exhibit heterogeneous combinations of genetic alterations that are the result of a process of natural selection. Through this process, cancer cells deregulate critical biological functions to establish the hallmarks of the transformed phenotype. The concept of convergent evolution in cancer implies that different genetic alterations can result in functionally similar outputs, which are likely to reflect an evolutionary advantage for the cancer cells with respect to their microenvironment.

Probing convergent evolution in molecular studies is technically challenging, in that typically few mutations can be induced in defined tumor models, raising the possibility that the observed effects are context-dependent. Here we resorted instead on a systematic analysis that extracted gene expression regulation concomitant with mutations in major cancer genes. Unexpectedly, we found that mutations in only 9 of 158 cancer genes were associated with substantial and recurrent changes on gene expression, and these were largely heterogeneous. Within this complexity, we could uncover a single node of convergence, a metabolic pathway that we termed AraX. AraX is a network of metabolic reactions that revolve around the metabolism of arachidonic acid and xenobiotics mediated by oxygen and glutathione. Our results showed that 9 frequently mutated genes in cancer converged in a significant association with transcriptional deregulation of AraX, more than with any other metabolic or biological pathway. This convergence is striking in that it occurs regardless of the cancer type and independent of the expression of a number of transcription factors. The survival analysis further corroborated that deregulation of AraX likely confers a context-independent selective advantage in cancer evolution.

Noteworthy, our analyses also unveiled other aspects about the convergence. First, among all genes, only in the case of two genes the corresponding expression changes were independently associated with at least 6 mutated genes both in the discovery and the validation cohort, namely HGD and ADH7. Remarkably, both genes are metabolic and linked to AraX. To our knowledge, HGD has never been implicated in cancer. Second, other metabolic processes showed patterns of convergence, although not as pronounced as for AraX. Prominently, many mutation-associated genes were related to protein glycosylation (see FIGS. 5C, and 6D).

Intriguingly, the fact that AraX is a transcriptionally regulated pathway of oxygen-consuming reactions could reflect a strategy by which cancer cells adapt to tumor hypoxia by regulating oxygen-dependent enzymes to compensate for reduced oxygen availability. Cancer mutations select independently for the deregulation of this pathway, potentially under the selective pressure of hypoxia. Based on these results, it is plausible to envision a universal modulation of the Keap1-Nrf3 pathway in the evolution of cancer, being this pathway the major cellular regulator of response to oxidative stress.

Collectively, our analysis suggests that in cancer there is convergent evolution on transcriptional deregulation of primarily the AraX pathway. An effective strategy to arrest cancer evolution is represented by either modulating the activity of the AraX pathway or the major regulatory axis associated with it, the Keap1-Nrf3 pathway, potentially using a multi-targeted approach, a strategy also advocated by network pharmacology.

### REFERENCES

1. Beisser, D., Klau, G. W., Dandekar, T., Muller, T., and Dittrich, M. T. (2010). BioNet: an R-Package for the functional analysis of biological networks. Bioinformatics 26, 1129-1130.
2. Drier, Y., Sheffer, M., and Domany, E. (2013). Pathway-based personalized analysis of cancer. Proceedings of the National Academy of Sciences of the United States of America 110, 6388-6393.
3. Durinck, S., Spellman, P. T., Birney, E., and Huber, W. (2009). Mapping identifiers for the integration of genomic datasets with the R/Bioconductor package biomaRt. Nature protocols 4, 1184-1191.
4. Friedman, J., Hastie, T., and Tibshirani, R. (2010). Regularization Paths for Generalized Linear Models via Coordinate Descent. J Stat Softw 33, 1-22.
5. Gao, J., Aksoy, B. A., Dogrusoz, U., Dresdner, G., Gross, B., Sumer, S. O., et al. (2013). Integrative analysis of complex cancer genomics and clinical profiles using the cBioPortal. Science signaling 6, p11.
6. Ideker, T., Ozier, O., Schwikowski, B., and Siegel, A. F. (2002). Discovering regulatory and signalling circuits in molecular interaction networks. Bioinformatics 18 Suppl 1, S233-240.
7. Law, C. W., Chen, C., Shi, W., and Smyth, G. K. (2013). Voom! precision weights unlock linear model analysis tools for RNA-seq read counts. In http://www.statsci.org/smyth/pubs/VoomPreprint.pdf.
8. Law, C. W., Chen, Y., Shi, W., and Smyth, G. K. (2014). Voom: precision weights unlock linear model analysis tools for RNA-seq read counts. Genome biology 15, R29.
9. Lawrence, M. S., Stojanov, P., Mermel, C. H., Robinson, J. T., Garraway, L. A., Golub, T. R., et al. (2014). Discovery and saturation analysis of cancer genes across 21 tumour types. Nature 505, 495-501.
10. Smyth, G. K. (2004). Linear models and empirical bayes methods for assessing differential expression in microarray experiments. Statistical applications in genetics and molecular biology 3, Article3.
11. Varemo, L., Nielsen, J., and Nookaew, 1. (2013). Enriching the gene set analysis of genome-wide data by incorporating directionality of gene expression and combining statistical hypotheses and methods. Nucleic acids research 41, 4378-4391.

TABLE A
Confusion matrices corresponding to classification of 443 normal vs.
4462 cancer based on the expression of one or more selected AraX genes.
Sample classification was performed using the random forest algorithm.
In each confusion matrix, rows correspond to the number of samples
classified by the algorithm as either Normal or Tumor, while columns
represent the number of samples that were actually normal or tumors
(except the third column, which represents the fraction of mis-classified
samples with the actual Normal vs. Tumor samples). The Fisher Exact
Test was used to compute the significance of each confusion matrix,
in that an odds-ratio >1 and a p-value <0.01 indicates an over-
representation of correctly classified samples compared to random
expectation.

| Classification using ADH1C | | | | Fisher | |
|---|---|---|---|---|---|
| | Normal | Tumor | class error | Test | |
| Normal | 37 | 406 | 0.91647856 | Odds-ratio | 0.93 |
| Tumor | 399 | 4063 | 0.08942178 | p-value | 0.72 |
| Classification using ADH1C and GPX3 | | | | Fisher | |
| | Normal | Tumor | class.error | Test | |
| Normal | 27 | 416 | 0.93905192 | Odds-ratio | 2.64 |
| Tumor | 107 | 4355 | 0.02398028 | p-value | 4.80E−05 |
| Classification using ADH1C and GPX3 | | | | | |
| and CDO1 | | | | Fisher | |
| | Normal | Tumor | class.error | Test | |
| Normal | 49 | 394 | 0.88939052 | Odds-ratio | 11.67 |
| Tumor | 47 | 4415 | 0.01053339 | p-value | 1.00E−16 |

TABLE B
log₂ fold-changes in the expression of each AraX gene (rows) in correspondence with the presence of a mutated gene in the tumor (columns).

| Gene | | | | | | | | | |
|---|---|---|---|---|---|---|---|---|---|
| Symbol | CTNNB1 | IDH1 | KEAP1 | NFE2L2 | NSD1 | PTEN | RB1 | STK11 | TP53 |
| FAAH2 | 0 | 0 | 0 | 0 | 0 | 0 | 0 | 0.664841252 | 0 |
| MBOAT2 | 0 | 0.607200356 | 0 | 0 | −0.749381524 | 0 | 0 | −0.828187089 | 0.597378419 |
| PLA2G2A | −0.984846659 | 0 | 0 | 0 | 0 | 0 | 0 | 0 | −1.31784875 |
| PLA2G4A | 0.94005356 | 0 | 0 | 0 | 0 | 0 | 0 | 1.204732285 | 0 |
| PLA2G4E | 0 | 0 | 0 | −0.832629798 | 0 | 0 | 0 | 0 | 0 |
| PLA2G10 | 0 | 0 | 0 | 0.667378053 | 0 | 0 | −0.88541099 | 1.200666483 | −0.904629888 |
| ELOVL2 | 0 | −1.980607776 | 0 | 0 | 0 | 0 | 0 | 0 | 0 |
| CYP2S1 | 0 | 0 | 0 | 0 | 0 | 0 | 0 | −0.991716918 | 0 |
| CYP4F11 | 0 | 0 | 2.371830007 | 3.655168738 | −0.938520549 | 0 | 0 | 0 | 0 |
| AKR1C3 | 0 | 0 | 2.139385174 | 2.987383639 | −0.874545486 | 0 | 0 | 1.017661248 | 0 |
| CBR1 | 0 | −0.639581803 | 0.793613196 | 1.177141186 | 0 | 0 | 0 | 0.605538109 | 0 |
| GSTM2 | 0 | 0 | 0 | 1.463479601 | 0 | 0 | 0 | 0 | 0 |
| GSTM3 | 0 | 0 | 0.89906434 | 1.805534833 | −0.601823481 | 0 | 0 | −1.2636177 | 0 |
| HPGDS | 0 | 0 | 0 | 0.587037144 | 0 | 0 | 0 | 0 | 0 |
| HPGD | 0.818919746 | 0 | −0.601803191 | 0 | 0 | 0 | 0 | 0 | −0.642447075 |
| PTGS1 | −0.7993257 | 0 | 0 | 0 | 0 | 0 | 0 | 0 | 0 |
| PTGES | 0 | 0 | 0 | 0 | 0 | 0 | 0 | 1.048581302 | 0 |
| ALOX15 | 0 | 0 | 0 | 0 | 0 | 0.845728289 | 0 | 0 | 0 |
| CYP4F3 | 0 | 0 | 2.204334292 | 4.029924172 | −1.17432704 | 0 | 0 | 0 | 0 |
| GGT6 | −0.771117285 | 0 | −0.697530129 | 0 | 0 | 0 | 0 | 0 | 0 |
| PTGR1 | 0 | 0 | 0.904628681 | 1.730669996 | 0 | 0 | 0 | 0 | 0 |
| GCLC | 0 | 0 | 0.976217154 | 1.286857118 | 0 | 0 | 0 | 0 | 0 |
| GCLM | 0 | 0 | 1.142950176 | 1.683340931 | 0 | 0 | 0 | 0 | 0 |
| GPX2 | 0 | 0 | 2.292346892 | 2.352299741 | 0 | 0 | 0 | 0 | −1.123160583 |
| GPX3 | −0.728377286 | 0 | 0 | 0 | 0 | 0 | −0.634921308 | 0.649915154 | 0 |
| GSR | 0 | 0 | 0.933873985 | 1.217937882 | 0 | 0 | 0 | 0 | 0 |
| OPLAH | −0.706691058 | 0 | 0 | 0 | 0 | 0 | 0 | 0 | 0 |
| CYP2W1 | 0 | 0 | 0 | −1.54220628 | 1.877450939 | 0 | 0 | 0 | 0 |
| CYP4B1 | 0 | 0 | 0 | 0 | 0 | 0 | 0 | 0 | −1.122718828 |
| CYP4X1 | 0 | 0 | −0.947674482 | 0 | 0 | 0 | 0 | 0 | 0 |
| CYP24A1 | 0 | 0 | 0.612149239 | −1.462752757 | 0 | 0 | 0 | 1.819588395 | −0.707173776 |
| CYP27A1 | 0 | −0.736647526 | 0 | 0 | 0 | 0 | 0 | 0 | 0 |
| CYP27B1 | 0 | 0 | 0 | −0.904101956 | 0 | 0 | 0 | 0 | 0 |
| CYP39A1 | 0 | 0 | 0 | 0.699547554 | −0.699518885 | 0 | 0 | 0 | 0 |
| HGD | −0.692668183 | −1.07898851 | 1.702885493 | 1.336956809 | 0 | 0.865952391 | 0 | 2.199463755 | 0 |
| MOXD1 | 0.720575592 | −1.246630965 | 0 | 0 | 0.66712777 | 0 | 0 | 0 | 0 |
| CDO1 | 0 | 0 | 0 | −0.586653304 | −0.640143659 | 0 | 0 | 0 | 0 |
| CP | −1.198826809 | 0 | 0 | 0 | 0 | 0 | 0 | 1.197420382 | 0 |
| CYP3A5 | 0.916308251 | 0 | 0 | −0.866501419 | 0 | 0 | 0 | 0 | −0.601099057 |
| ADH1C | 0 | 0 | 0 | 1.091612504 | 0 | 0 | 0 | 0 | −0.707013961 |
| ADH6 | 0 | 0 | 0 | 0 | 0 | 0 | 0 | 0 | −0.799541689 |
| ADH7 | 0 | 0 | 1.454193935 | 3.108640836 | −1.126577207 | 0 | 1.046946984 | −2.536027757 | −1.056514104 |
| ADHFE1 | 0 | 0 | 0 | 0 | −0.984232077 | 0 | 0 | 0 | 0 |
| FMO3 | 0 | 0.792224809 | 0 | 0 | 0 | 0 | 0 | 0 | 0 |
| FMO4 | 0 | 0 | 0 | 0 | −0.740312277 | 0 | 0 | 0 | 0 |
| FMO5 | 0 | 0 | 0 | 0 | 0 | 0 | 0 | 0 | −0.736940082 |
| AKR1B15 | −1.31729609 | 0 | 1.552215915 | 1.746039773 | 0 | 0 | 0 | 0 | 0 |
| AKR1B10 | 0 | 0 | 2.244979912 | 3.964605795 | −1.516369905 | 0 | 0 | 0 | 0 |
| AKR1C1 | 0 | 0 | 2.940243553 | 3.671711735 | −0.906169718 | 0 | −0.715368152 | 1.268422351 | −0.728686568 |
| AKR1C2 | 0 | 0 | 2.811215621 | 3.257929361 | 0 | 0 | −0.815957303 | 2.134747 | −0.663968674 |
| NQO1 | 0 | 0 | 1.623429625 | 2.096366281 | 0 | 0.787660938 | 0 | 0.790838317 | 0 |
| NQO2 | 0 | 0 | 0 | 0 | 0 | 0 | 0 | 0.657270426 | 0 |
| CBR3 | 0 | 0 | 0.833046419 | 1.73352929 | 0 | 0 | 0 | 0 | 0 |
| ALDH3A1 | 0 | 0 | 1.699073159 | 3.063646184 | 0 | 0 | 0 | 0 | −0.779762291 |
| ALDH3A2 | 0 | 0 | 0 | 0.975446485 | 0 | 0 | 0 | 0 | 0 |
| ALDH3B1 | 0 | −0.63581752 | 0 | 0 | 0 | 0 | 0 | 1.580804194 | 0 |
| AOC1 | −1.024071593 | 0 | 0 | 0 | −0.586732897 | 0 | 0 | 1.344882995 | 0 |
| MAOB | 0.677250537 | −1.49436905 | 0 | 0 | 0 | 0 | 0 | 0 | 0 |
| CES1 | 0 | 0 | 2.188208693 | 3.944251065 | −1.431707828 | 0 | 0 | 0 | 0 |
| EPHX1 | 0 | 0 | 0.734147151 | 1.262515762 | 0 | 0 | 0 | 0 | 0 |
| GSTA2 | 0 | 0 | 0 | 1.88334365 | 0 | 0 | 0 | 0 | 0 |
| GSTM1 | 0 | 0 | 0 | 2.629770868 | 0 | 0 | 0 | 0 | 0 |
| GSTM4 | 0 | 0 | 0 | 1.064274574 | 0 | 0 | 0 | 0 | 0 |
| MGST1 | 0 | 0 | 0 | 1.034132771 | 0 | 0 | 0 | 0 | 0 |
| UGT1A1 | 0 | 0 | 1.783397767 | 2.464145932 | 0 | 0 | 0 | 0 | 0 |
| UGT1A6 | 0 | 0 | 1.22045589 | 2.095200126 | 0 | 0 | 0 | 0 | −0.720676112 |
| SULT1A1 | 0 | 0 | 0 | 0.833860445 | 0 | 0 | 0 | 0 | 0 |
| SULT1A2 | 0 | 0 | 0 | 0.591055591 | 0 | 0 | 0 | 1.010655715 | 0 |
| SULT1A4 | 0 | 0 | 0 | 0 | 0 | 0 | 0 | 0.637088049 | 0 |
| ACSL5 | 0.901238898 | 0 | 0 | 0 | 0 | 0 | −0.70196394 | 0 | 0 |
| SLCO1B3 | 0 | 0 | 0 | 0.929176125 | 0 | 0 | 0 | 2.427769853 | 0 |
| SLCO2A1 | 0 | 0 | 0 | −0.604011468 | 0 | 0 | 0 | 0 | 0 |
| ABCC1 | 0 | 0 | 0.617396723 | 0.901197316 | 0 | 0 | 0 | 0 | 0 |
| ABCC2 | 0 | 0 | 1.45981815 | 1.629831892 | 0 | −0.59499652 | 0 | 1.693517186 | 0 |
| ABCC3 | 0 | −0.908331239 | 0.826278245 | 1.488876075 | 0 | 0 | 0 | 0 | −0.701561655 |
| ALOX5 | 0 | −0.622368133 | 0 | 0 | 0 | 0 | 0 | 0 | 0 |
| CYP2E1 | 0 | 0.461446615 | 0 | 0 | 0 | −0.450699052 | −0.673433523 | 0 | 0 |
| LTC4S | 0 | −0.750287505 | 0 | 0 | 0 | 0 | −0.520841266 | 0 | 0 |
| PLA2G6 | 0 | 0 | −0.519669563 | 0 | 0 | 0 | −0.487948561 | 0 | 0 |
| PLA2G12A | 0 | 0 | 0 | 0 | 0 | 0 | 0 | 0.454249309 | 0 |
| PTGS2 | 0 | 0 | 0 | −1.205138722 | 0 | 1.07030287 | 0 | 0 | 0 |
| GSTO1 | 0 | 0 | 0.611614834 | 0 | 0 | 0 | 0 | 0 | 0 |
| GSTO2 | 0.977491953 | 0.433984558 | 0 | 0 | 0 | 0 | 0 | 1.198819603 | 0 |
| FMO1 | −1.161295512 | −0.552477811 | −0.913774167 | 0.685245295 | 0 | 0 | 0 | 0 | 0 |

1. A method of screening for cancer in a subject, said method comprising
determining the level in a sample of an expression product of one or more genes selected from the group consisting of:
ADH1C, FAAH2, MBOAT2, PLA2G2A, PLA2G4A, PLA2G4E, PLA2G10, ELOVL2, CYP2S1, CYP4F11, AKR1C3, CBR1, GSTM2, GSTM3, HPGDS, HPGD, PTGS1, PTGES, ALOX15, CYP4F3, GGT6, PTGR1, GCLC, GCLM, GPX2, GPX3, GSR, OPLAH, CYP2W1, CYP4B1, CYP4X1, CYP24A1, CYP27A1, CYP27B1, CYP39A1, HGD, MOXD1, CDO1, CP, CYP3A5, ADH6, ADH7, ADHFE1, FMO3, FMO4, FMO5, AKR1B15, AKR1B10, AKR1C1, AKR1C2, NQO1, NQO2, CBR3, ALDH3A1, ALDH3A2, ALDH3B1, AOC1, MAOB, CES1, EPHX1, GSTA2, GSTM1, GSTM4, MGST1, UGT1A1, UGT1A6, SULT1A1, SULT1A2, SULT1A4, ACSL5, SLCO1B3, SLCO2A1, ABCC1, ABCC2, ABCC3, ALOX5, CYP2E1, LTC4S, PLA2G6, PLA2G12A, PTGS2, GSTO1, GSTO2 and FMO1
and/or
determining the level in a sample of a metabolite related to an expression product of one or more of said genes;
wherein said sample has been obtained from said subject; and
wherein an altered level in said sample of the expression product of one or more of said genes and/or of a metabolite related to an expression product of one or more of said genes in comparison to a control level is indicative of cancer in said subject.

2. The method of claim 1, wherein the level of an expression product of one or more of said genes is determined.

3. The method of claim 1, wherein the level of an expression product, or a related metabolite, of ADH1C, GPX3 and/or CDO1 is determined, preferably wherein the level of an expression product of ADH1C, or a related metabolite, is determined.

4. The method of claim 1, wherein the level of an expression product, or a related metabolite, of HGD, ADH7 and/or ALDH3A1 is determined, preferably wherein the level of an expression product of HGD and/or ADH7, or a related metabolite, is determined.

5. The method of claim 1, wherein said sample comprises a mutated version of one or more genes selected from the group consisting of CTNNB1, IDH1, KEAP1, NFE2L2, NSD1, PTEN, RB1, STK11 and TP53.

6. The method of claim 1, wherein if a mutation in the CTNNB1 gene is present, an alteration in the level of an expression product of one or more genes selected from the group consisting of PLA2G4A, HPGD, MOXD1, CYP3A5, MAOB, ACSL5, GSTO2, PLA2G2A, PTGS1, GGT6, GPX3, OPLAH, HGD, CP, AKR1B15, AOC1 and FMO1 or of a metabolite related to one or more of said genes is indicative of cancer in said subject; or
wherein if a mutation in the IDH1 gene is present, an alteration in the level of an expression product of one or more genes selected from the group consisting of MBOAT2, FMO3, CYP2E1, GSTO2, ELOVL2, CBR1, CYP27A1, HGD, MOXD1, ALDH3B1, MAOB, ABCC3, ALOX5, LTC4S and FMO1 or of a metabolite related to one or more of said genes is indicative of cancer in said subject; or
wherein if a mutation in the KEAP1 gene is present, an alteration in the level of an expression product of one or more genes selected from the group consisting of CYP4F11, AKR1C3, CBR1, GSTM3, CYP4F3, PTGR1, GCLC, GCLM, GPX2, GSR, CYP24A1, HGD, ADH7, AKR1B15, AKR1B10, AKR1C1, AKR1C2, NQO1, CBR3, ALDH3A1, CES1, EPHX1, UGT1A1, UGT1A6, ABCC1, ABCC2, ABCC3, GSTO1 HPGD, GGT6, CYP4X1, PLA2G6 and FMO1 or of a metabolite related to one or more of said genes is indicative of cancer in said subject; or
wherein if a mutation in the NFE2L2 gene is present, an alteration in the level of an expression product of one or more genes selected from the group consisting of PLA2G10, CYP4F11, AKR1C3, CBR1, GSTM2, GSTM3, HPGDS, CYP4F3, PTGR1, GCLC, GCLM, GPX2, GSR, CYP39A1, HGD, ADH1C, ADH7, AKR1B15, AKR1B10, AKR1C1, AKR1C2, NQO1, CBR3, ALDH3A1, ALDH3A2, CES1, EPHX1, GSTA2, GSTM1, GSTM4, MGST1, UGT1A1, UGT1A6, SULT1A1, SULT1A2, SLCO1B3, ABCC1, ABCC2, ABCC3, FMO1, PLA2G4E, CYP2W1, CYP24A1, CYP27B1, CDO1, CYP3A5, SLCO2A1 and PTGS2 or of a metabolite related to one or more of said genes is indicative of cancer in said subject; or
wherein if a mutation in the NSD1 gene is present, an alteration in the level of an expression product of one or more genes selected from the group consisting of CYP2W1, MOXD1, MBOAT2, CYP4F11, AKR1C3, GSTM3, CYP4F3, CYP39A1, CDO1, ADH7, ADHFE1, FMO4, AKR1B10, AKR1C1, AOC1 and CES1 or of a metabolite related to one or more of said genes is indicative of cancer in said subject; or
wherein if a mutation in the PTEN gene is present, an alteration in the level of an expression product of one or more genes selected from the group consisting of ALOX15, HGD, NQO1, PTGS2, ABCC2 and CYP2E1 or of a metabolite related to one or more of said genes is indicative of cancer in said subject; or
wherein if a mutation in the RB1 gene is present, an alteration in the level of an expression product of one or more genes selected from the group consisting of ADH7, PLA2G10, GPX3, AKR1C1, AKR1C2, ACSL5, CYP2E1, LTC4S and PLA2G6 or of a metabolite related to one or more of said genes is indicative of cancer in said subject; or
wherein if a mutation in the STK11 gene is present, an alteration in the level of an expression product of one or more genes selected from the group consisting of FAAH2, PLA2G4A, PLA2G10, AKR1C3, CBR1, PTGES, GPX3, CYP24A1, HGD, CP, AKR1C1, AKR1C2, NQO1, NQO2, ALDH3B1, AOC1, SULT1A2, SULT1A4, SLCO1B3, ABCC2, PLA2G12A, GSTO2, MBOAT2, CYP2S1, GSTM3 and ADH7 or of a metabolite related to one or more of said genes is indicative of cancer in said subject; or
wherein if a mutation in the TP53 gene is present, an alteration in the level of an expression product of one or more genes selected from the group consisting of MBOAT2, PLA2G2A, PLA2G10, HPGD, GPX2, CYP4B1, CYP24A1, CYP3A5, ADH1C, ADH6, ADH7, FMO5, AKR1C1, AKR1C2, ALDH3A1, UGT1A6 and ABCC3 or of a metabolite related to one or more of said genes is indicative of cancer in said subject.

7. The method of claim 1,
wherein the level of an expression product, or related metabolite, of more than one of said genes is determined; or
wherein the level of an expression product, or related metabolite, of 2, 3, 4, 5, 6 or 7 of said genes is determined.

8. The method of claim 1, wherein the level of expression products of ADH1C and GPX3 are determined or wherein the level of expression products of ADH1C, GPX3 and CDO1 are determined.

9. The method of claim 1,
wherein said method comprises determining the level of an expression product of ADH1C in combination with determining the level of an expression product, or related metabolite, of at least one other of said genes; and/or
wherein said method comprises determining the level of an expression product of GPX3 in combination with determining the level of an expression product, or related metabolite, of at least one other of said genes; and/or
wherein said method comprises determining the level of an expression product of CDO1 in combination with determining the level of an expression product, or related metabolite, of at least one other of said genes.

10. The method of claim 1, wherein said expression product is an mRNA molecule, or a fragment thereof.

11. The method of claim 1, wherein said expression product is a polypeptide, or a fragment thereof.

12. The method of claim 1,
wherein the level of the expression product of one or more of said genes is determined by a primer-directed nucleic acid amplification reaction, by a microarray or by RNA-seq; or
wherein the level of a metabolite related to an expression product of one or more of said genes is determined by gas/liquid chromatography coupled with mass-spectrometry.

13. The method of claim 1, wherein said method is used for diagnosing cancer, for the prognosis of cancer, for monitoring the progression of cancer, for determining the clinical severity of cancer, for predicting the response of a subject to therapy, for determining the efficacy of a therapeutic regime being used to treat cancer, for detecting the recurrence of cancer, for distinguishing between indolent and aggressive cancer, or for predicting the survival prospects for a cancer patient.

14. The method of claim 1, wherein said subject is a human subject.

15. The method of claim 1, wherein said cancer is colon cancer, head and neck cancer, lung cancer, uterine cancer, oesophageal cancer, bladder cancer, glioblastoma multiforme, kidney cancer, glioma, ovarian cancer, rectal cancer or pancreatic cancer.

16. The method of claim 1, further comprising a step of treating cancer by therapy or surgery.

17. The method of claim 1, further comprising a step of processing said sample.

18. The method of claim 1, wherein said subject is a subject at risk of developing cancer, or a subject at risk of the occurrence of cancer or a subject having, or suspected of having, cancer.

19. A method for treating cancer, which method comprises administering to a subject in need thereof a therapeutically effective amount of an agent which modulates the level and/or activity of an expression product, or related metabolite, of one or more genes selected from the group consisting of ADH1C, FAAH2, MBOAT2, PLA2G2A, PLA2G4A, PLA2G4E, PLA2G10, ELOVL2, CYP2S1, CYP4F11, AKR1C3, CBR1, GSTM2, GSTM3, HPGDS, HPGD, PTGS1, PTGES, ALOX15, CYP4F3, GGT6, PTGR1, GCLC, GCLM, GPX2, GPX3, GSR, OPLAH, CYP2W1, CYP4B1, CYP4X1, CYP24A1, CYP27A1, CYP27B1, CYP39A1, HGD, MOXD1, CDO1, CP, CYP3A5, ADH6, ADH7, ADHFE1, FMO3, FMO4, FMO5, AKR1B15, AKR1B10, AKR1C1, AKR1C2, NQO1, NQO2, CBR3, ALDH3A1, ALDH3A2, ALDH3B1, AOC1, MAOB, CES1, EPHX1, GSTA2, GSTM1, GSTM4, MGST1, UGT1A1, UGT1A6, SULT1A1, SULT1A2, SULT1A4, ACSL5, SLCO1B3, SLCO2A1, ABCC1, ABCC2, ABCC3, ALOX5, CYP2E1, LTC4S, PLA2G6, PLA2G12A, PTGS2, GSTO1, GSTO2 and FMO1.

20. The method of claim 19, wherein said method comprises administering to a subject in need thereof a therapeutically effective amount of an agent which modulates the level and/or activity of an expression product, or related metabolite, of one or more genes selected from the group consisting of ADH7, GSTM3, ABCC2, PTGR1 and CBR3.

21. A kit for the screening of cancer which comprises an agent suitable for determining the level of an expression product, or related metabolite, of one or more of the genes selected from the group consisting of ADH1C, FAAH2, MBOAT2, PLA2G2A, PLA2G4A, PLA2G4E, PLA2G10, ELOVL2, CYP2S1, CYP4F11, AKR1C3, CBR1, GSTM2, GSTM3, HPGDS, HPGD, PTGS1, PTGES, ALOX15, CYP4F3, GGT6, PTGR1, GCLC, GCLM, GPX2, GPX3, GSR, OPLAH, CYP2W1, CYP4B1, CYP4X1, CYP24A1, CYP27A1, CYP27B1, CYP39A1, HGD, MOXD1, CDO1, CP, CYP3A5, ADH6, ADH7, ADHFE1, FMO3, FMO4, FMO5, AKR1B15, AKR1B10, AKR1C1, AKR1C2, NQO1, NQO2, CBR3, ALDH3A1, ALDH3A2, ALDH3B1, AOC1, MAOB, CES1, EPHX1, GSTA2, GSTM1, GSTM4, MGST1, UGT1A1, UGT1A6, SULT1A1, SULT1A2, SULT1A4, ACSL5, SLCO1B3, SLCO2A1, ABCC1, ABCC2, ABCC3, ALOX5, CYP2E1, LTC4S, PLA2G6, PLA2G12A, PTGS2, GSTO1, GSTO2 and FMO1, or fragments thereof, in a sample.
